(19) United States
(12) Patent Application Publication
(10) Pub. No.: US 2021/0290787 A1
(43) Pub. Date: Sep. 23, 2021

(54) MACROCYCLE CONJUGATES

(71) Applicant: LUMIPHORE, INC., Berkeley, CA (US)

(72) Inventors: Darren MAGDA, San Leandro, CA (US); David TATUM, Berkeley, CA (US); Nathaniel G. BUTLIN, Pacifica, CA (US)

(21) Appl. No.: 17/253,550

(22) PCT Filed: Jun. 19, 2019
(86) PCT No.: PCT/US2019/038057

§ 371 Date: Dec. 17, 2020

Related U.S. Application Data
(60) Provisional application No. 62/686,997, filed on Jun. 19, 2018.

(51) Int. Cl. A61K 51/08; A61K 49/00; A61K 47/64

(52) U.S. Cl. CPC A61K 51/088; A61K 47/64; A61K 49/0056

(57) ABSTRACT

The invention relates to chemical compounds and complexes that can be used in therapeutic and diagnostic applications. In various embodiments, the invention described herein pertains to the diagnosis, imaging, and/or treatment of pathogenic cell populations. In particular, the invention described herein pertains to the diagnosis, imaging, and/or treatment of diseases caused by PSMA express-ing cells, such as prostate cancer cells, using compounds capable of targeting PSMA expressing cells.

### CROSS-REFERENCES TO RELATED APPLICATIONS

The present application claims priority to U.S. Provisional Application No. 62/686,997, filed Jun. 19, 2018; the disclosure of which is hereby incorporated by reference in its entirety for all purposes.

### STATEMENT OF GOVERNMENT RIGHTS

This invention was made with Government support under Grant No. IIP-1353612 awarded by the Small Business Administration. The Government has certain rights in the invention.

### FIELD OF THE INVENTION

The invention relates to chemical compounds and complexes that can be used in therapeutic and diagnostic applications.

### BACKGROUND OF THE INVENTION

At least 1 million men suffer from prostate cancer and it's estimated that the disease will strike one in six U.S. men between the ages of 60 and 80. There are more than 300,000 new cases of prostate cancer diagnosed each year. Prostate cancer will affect one in six men in the United States, and the mortality from the disease is second only to lung cancer. An estimated $2 billion is currently spent worldwide on surgical, radiation, drug therapy and minimally invasive treatments, $1 billion of the spending in the U.S. There is presently no effective therapy for relapsing, metastatic, androgen-independent prostate cancer. New agents that will enable rapid visualization of prostate cancer and specific targeting to allow radiotherapy present are needed.

N-acetylated alpha-linked acidic dipeptidase (NAALADase), also known as glutamate carboxypeptidase II (GCPII) is a neuropeptidase which cleaves N acetylaspartyl-glutamate (NAAG) into N-acetylaspartate and glutamate in the nervous system, see below, depicting hydrolytic cleavage of NAAG by NAALDase through the tetrahedral intermediate. The enzyme is a type II protein of the co-catalytic class of metallopeptidases, containing two zinc atoms in the active site.

Independent of its characterization in the nervous system, one form of NAALADase was shown to be expressed at high levels in human prostatic adenocarcinomas and was designated the prostate-specific membrane antigen (PSMA). The NAALADase/PSMA gene is known to produce multiple mRNA splice forms and based on previous immunohistochemical evidence, it has been assumed that the human brain and prostate expressed different isoforms of the enzyme.

Human prostate-specific membrane antigen (PSMA), also known as folate hydrolase I (FOLHI), is a trans-membrane, 750 amino acid type II glycoprotein which is primarily expressed in normal human prostate epithelium but is upregulated in prostate cancer, including metastatic disease. PSMA is a unique exopeptidase with reactivity toward poly-gamma-glutamated folates, capable of sequentially removing the poly-gamma-glutamyl termini. Since PSMA is expressed by virtually all prostate cancers and its expression is further increased in poorly differentiated, metastatic and hormone-refractory carcinomas, it is a very attractive target for prostate imaging and therapy. Developing ligands that interact with PSMA and carry appropriate radionuclides may provide a promising and novel targeting option for the detection, treatment and management of prostate cancer.

The radio-immunoconjugate form of the anti-PSMA monoclonal antibody (mAb) 7E1 1, known as the PROSTASCINT scan, is currently being used to diagnose prostate cancer metastasis and recurrence. Early promising results from various Phase I and II trials have utilized PSMA as a therapeutic target. PROSTASCINT targets the intracellular domain of PSMA and is thought to bind mostly necrotic portions of prostate tumor. More recently, monoclonal antiobodies have beed developed that bind to the extracellular domain of PSMA and have been radiolabeled and shown to accumulate in PSMA-positive prostate tumor models in animals.

While monoclonal antibodies hold promise for tumor detection and therapy, there have been limited clinical successes outside of lymphoma because of their low permeability in solid tumors. Low molecular weight mimetics, with higher permeability in solid tumors will have a definite advantage in obtaining high percent per gram and a high percentage of specific binding.

Inhibitors of prostate specific membrane antigen (PSMA) attached to chelating molecules have been reported by Matthias Eder, et al., WO2015055318, by John W. Babich, et al., WO2008058192 and WO2013022797, by Martin G. Pomper, et al., WO2010108125, by Clifford Berkman, et al., WO2018031809, and are incorporated by reference. Fluorescent PSMA ligands and uses thereof have been reported by James Basilion, US20150366968A1. Phosphoramidate derivatives of hydroxysteroids as inhibitors of PSMA have also been reported (Wu, L. Y., et al., Bioorg. Med. Chem. Let. 2008, 18, 281-284). A conjugate of a decapeptide targeting luteinizing hormone releasing hormone (LHRH) receptor (also known as gonadotropin releasing hormone receptor, GNRHR) with doxorubicin has been reported (Schally, A. V.; Nagy, A. Cancer chemotherapy based on targeting of cytotoxic peptide conjugates to their receptors on tumors. Eur. J. of Endocrinol., 1999, 141, 1-14).

Conjugates between an inhibitor of PSMA or LHRH and a versatile chelating agent capable of forming robust, stable chelates with metal ions would represent a significant advance in both treatment and diagnosis of primary and metastatic tumors.

### SUMMARY OF THE INVENTION

In various embodiments, the present invention provides ligands targeting species overexpressed in primary or metastatic tumors. Described herein are compounds capable of binding to a cell-surface receptor, such as PSMA and/or LHRH and/or somatostatin receptor. According to certain embodiments of the invention, the compounds of the invention are conjugates of chelating moieties that complex with certain metal ions with high affinity under convenient (e.g., therapeutically relevant) conditions. In some instances, these chelating moieties have the additional property of sensitizing metal ion luminescence which may be useful for measuring the receptor binding activity of the conjugates during development as, for example, radiopharmaceuticals.

An exemplary compound of the invention is a PSMA-targeting peptide or peptidomimetic core coupled to a chelating agent-metal ion moiety (“complex”) serving as an imaging reporter or a therapeutic radiotracer. An exemplary embodiment provides a compound having the structure:

CH-L⁰-TA

where CH is a chelating agent comprising one or more chelating moieties capable of binding and complexing a metal ion. An exemplary chelating agent has a formula selected from:

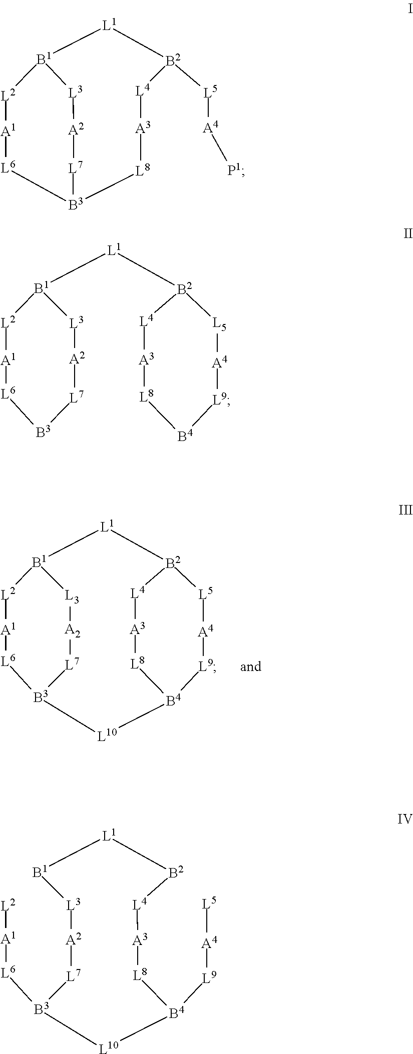

wherein
B¹, B², and B³ are independently selected from N and C;
L¹, L², L³, L⁴, L⁵, L⁶, L⁷, and L⁸ are independently selected from substituted or unsubstituted alkyl, and substituted or unsubstituted heteroalkyl;
A¹, A², and A³ are members independently selected from:

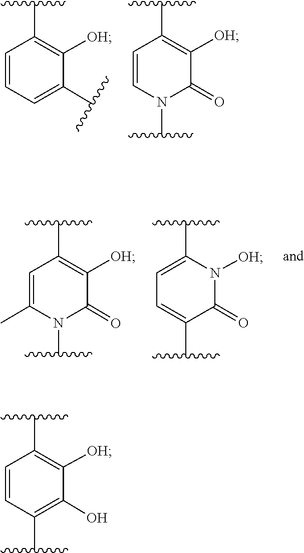

and
P¹ is a member selected from:

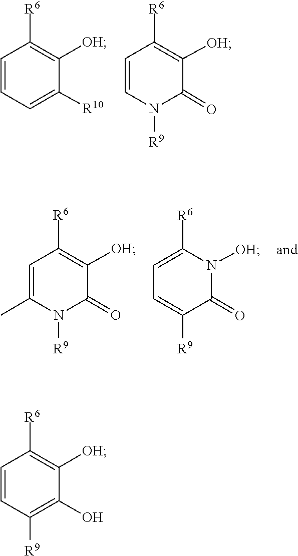

wherein R⁶, R⁹, and R¹⁰ are as defined herein, with the proviso that R⁶, R⁹, or R¹⁰ is a bond to L⁵; L^{o} is a linker joining the chelating agent (CH) to the targeting agent (TA), which is a member selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted heteroalkyl, substituted or unsubstituted heterocycloalkyl, substituted or unsubstituted aryl, substituted or unsubstituted arylalkyl, substituted or unsubstituted biaryl, substituted or unsubstituted heteroaryl, and a substituted or unsubstituted polycyclic ring system; and TA is a targeting moiety selected from a compound binding to PSMA and/or LHRH.

In some embodiments, one of L¹, L², L³, L⁴, L⁵, L⁶, L⁷, L⁸, and P¹ is substituted with L^{o} and, therethrough to TA. In some embodiments, P¹ is substituted with L^{o}. In some embodiments, L⁵ is substituted with L^{o}. In some embodiments, L² is substituted with L^{o}.

The invention also provides methods of using the compounds of the invention in the manufacture of pharmaceutal formulations, and for the diagnosis, imaging and treatment of diseases, including both primary and metastatic prostate cancer lesions.

Other embodiments, objects and uses of the invention will be apparent from the description that follows.

### BRIEF DESCRIPTION OF DRAWINGS

FIG. 1A-FIG. 1C. The HPLC chromatograms collected at 315 nm for a) the chelating agent starting material, b) the NHS reaction product, and c) the NHS reaction product dissolved in DMF at RT for 1 day. Note that all of the desired NHS reaction product is consumed by unknown side reactions within the 1 day incubation.

FIG. 2A-FIG. 2C. The HPLC chromatograms collected at 315 nm for a) the Ca starting material, b) the⋅Ca.NHS reaction product, and c) the Ca.NHS reaction product dissolved in DMF at RT for 1 day. Note that, unlike the previous experiment, the Ca.NHS reaction product remains intact after incubation for 1 day.

FIG. 3A-FIG. 3B. The HPLC chromatograms collected at 315 nm for a Ca.NHS (top) and Mg.NHS (bottom) dissolved in DMF at RT for 1 day.

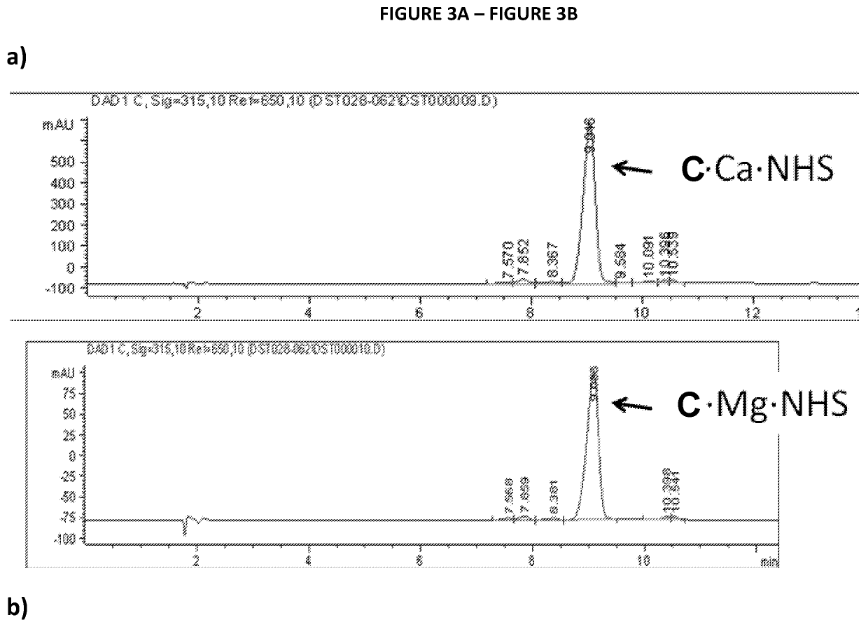

### DESCRIPTION OF EMBODIMENTS

### 1. Introduction

Described herein are compounds capable of binding to a cell-surface receptor, such as PSMA and/or LHRH and/or somatostatin receptor (e.g., ((Tyr3)-octreotate), and conjugates of these compounds with chelating agents, and chelating agents complexing a metal ion. Also described herein are compounds capable of targeting PSMA for delivery of diagnostic, imaging, and therapeutic agents. Also described herein are compounds and compositions, and methods and uses thereof for diagnosing, imaging, and treating diseases caused by pathogenic populations of cells that express, or overexpress, PSMA and/or LHRH.

PSMA and LHRH inhibitors appropriate as members of a conjugation pair are known in the art. See, for example WO2013/022797, and US20180085478. Exemplary classes of inhibitors include ureas, phosphoramidates and thiols. Ee, e.g., Gourni et al., Molecules 2017; 22: 523.

In another embodiment, pharmaceutical compositions containing one or more of the compounds and/or conjugates are also described herein. In one aspect, the compositions are in bulk form and are suitable for preparing unit doses, unit dosage forms, and the like that may be included in the uses and/or methods described herein. In another aspect, the compositions include a therapeutically effective amount of the one or more compounds for diagnosis, imaging, and/or treatment of diseases caused by PSMA expressing cells in a patient. Illustrative compositions include unit doses, unit dosage forms, and the like. It is to be understood that the compositions may include other components and/or ingredients, including, but not limited to, other therapeutically active compounds, and/or one or more carriers, and/or one or more diluents, and/or one or more excipients, and the like. In another embodiment, methods for using the compounds and pharmaceutical compositions for diagnosis, imaging, and/or treatment of diseases caused by PSMA expressing cells in a patient are also described herein. In one aspect, the methods include the step of administering one or more of the compounds and/or compositions described herein to the patient.

In another embodiment, uses of the compounds and compositions in the manufacture of a medicament for diagnosis, imaging, and/or treatment of diseases caused by PSMA expressing cells in a patient are also described herein. In one aspect, the medicaments include a therapeutically effective amount of the one or more compounds and/or compositions described herein.

### 2. Definitions

Where substituent groups are specified by their conventional chemical formulae, written from left to right, they optionally equally encompass the chemically identical substituents, which would result from writing the structure from right to left, e.g., —CH₂O— is intended to also recite —OCH₂—.

The term “alkyl”, by itself or as part of another substituent, means a straight or branched chain hydrocarbon, which may be fully saturated, mono- or polyunsaturated and includes mono-, di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl” can refer to “alkylene”, which by itself or as part of another substituent means a divalent radical derived from an alkane, as exemplified, but not limited, by —CH₂CH₂CH₂CH₂—. Typically, an alkyl (or alkylene) group will have from 1 to 30 carbon atoms. A “lower alkyl” or “lower alkylene” is a shorter chain alkyl or alkylene group, generally having eight or fewer carbon atoms. In some embodiments, alkyl refers to an alkyl or combination of alkyls selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl. In some embodiments, alkyl refers to C₁-C₂₅ alkyl. In some embodiments, alkyl refers to C₁-C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The term “heteroalkyl,” by itself or in combination with another term, means an alkyl in which one or more carbons are replaced with one or more heteroatoms selected from the group consisting of O, N, Si and S, (preferably O, N and S), wherein the nitrogen and sulfur atoms may optionally be oxidized and the nitrogen heteroatom may optionally be quaternized. The heteroatoms 0, N, Si and S may be placed at any interior position of the heteroalkyl group or at the position at which the alkyl group is attached to the remainder of the molecule. In some embodiments, depending on whether a heteroatom terminates a chain or is in an interior position, the heteroatom may be bonded to one or more H or substituents such as (C₁, C₂, C₃, C₄, C₅ or C₆) alkyl according to the valence of the heteroatom. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. No more than two heteroatoms may be consecutive, as in, for example, —CH₂—NH—OCH₃ and —CH₂—O—Si(CH₃)₃, and in some instances, this may place a limit on the number of heteroatom substitutions. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. The designated number of carbons in heteroforms of alkyl, alkenyl and alkynyl includes the heteroatom count. For example, a (C₁, C₂, C₃, C₄, C₅ or C₆) heteroalkyl will contain, respectively, 1, 2, 3, 4, 5 or 6 atoms selected from C, N, O, Si and S such that the heteroalkyl contains at least one C atom and at least one heteroatom, for example 1-5 C and 1 N or 1-4 C and 2 N. Further, a heteroalkyl may also contain one or more carbonyl groups. In some embodiments, a heteroalkyl is any C₂-C₃₀ alkyl, C₂-C₂₅ alkyl, C₂-C₂₀ alkyl, C₂-C₁₅ alkyl, C₂-C₁₀ alkyl or C₂-C₆ alkyl in any of which one or more carbons are replaced by one or more heteroatoms selected from O, N, Si and S (or from O, N and S). In some embodiments, each of 1, 2, 3, 4 or 5 carbons is replaced with a heteroatom.

The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The terms “cycloalkyl” and “heterocycloalkyl”, by themselves or in combination with other terms, refer to cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means a polyunsaturated, aromatic substituent that can be a single ring or optionally multiple rings (preferably 1, 2 or 3 rings) that are fused together or linked covalently. In some embodiments, aryl is a 3, 4, 5, 6, 7 or 8 membered ring, which is optionally fused to one or two other 3, 4, 5, 6, 7 or 8 membered rings. The term “heteroaryl” refers to aryl groups (or rings) that contain 1, 2, 3 or 4 heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

In some embodiments, any of alkyl, heteroalkyl, cycloalkyl, heterocycloalkyl, aryl and heteroaryl is optionally substituted. That is, in some embodiments, any of these groups is substituted or unsubstituted. In some embodiments, substituents for each type of radical are selected from those provided below.

Substituents for the alkyl, heteroalkyl, cycloalkyl and heterocycloalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents”. In some embodiments, an alkyl group substituent is selected from -halogen, —OR′, ═O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″ ″, —NR—C(NR′R″)═NR′, —S(O)R′, —S(O)₂R′, —S(O)₂NR′R″, —NRSO₂R′, —CN and —NO₂ in a number ranging from zero to (2m′+1), where m′ is the total number of carbon atoms in such radical. In one embodiment, R′, R″, R′″ and R″″ are each independently selected from hydrogen, alkyl (e.g., C₁, C₂, C₃, C₄, C₅ and C₆ alkyl). In one embodiment, R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. In one embodiment, R′, R″, R′″ and R″″ are each independently selected from hydrogen, alkyl, heteroalkyl, cycloalkyl, heterocycloalkyl, aryl, heteroaryl, alkoxy, thioalkoxy groups, and arylalkyl. When R′ and R″ are attached to the same nitrogen atom, they can be combined with the nitrogen atom to form a 5-, 6-, or 7-membered ring. For example, —NR′R″ can include 1-pyrrolidinyl and 4-morpholinyl. In some embodiments, an alkyl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Similar to the substituents described for the alkyl radical, substituents for the aryl and heteroaryl groups are generically referred to as “aryl group substituents”. In some embodiments, an aryl group substituent is selected from -halogen, —OR′, ═O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″ ″, —NR—C(NR′R″)═NR′, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. In some embodiments, R′, R″, R′″ and R″″ are independently selected from hydrogen and alkyl (e.g., C₁, C₂, C₃, C₄, C₅ and C₆ alkyl). In some embodiments, R′, R″, R′″ and R″″ are independently selected from hydrogen, substituted or unsubstituted alkyl, substituted or unsubstituted heteroalkyl, substituted or unsubstituted aryl and substituted or unsubstituted heteroaryl. In some embodiments, R′, R″, R′″ and R″″ are independently selected from hydrogen, alkyl, heteroalkyl, aryl and heteroaryl. In some embodiments, an aryl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “acyl” refers to a species that includes the moiety —C(O)R, where R has the meaning defined herein. Exemplary species for R include H, halogen, substituted or unsubstituted alkyl, substituted or unsubstituted aryl, substituted or unsubstituted heteroaryl, and substituted or unsubstituted heterocycloalkyl. In some embodiments, R is selected from H and (C₁-C₆)alkyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. In some embodiments, halogen refers to an atom selected from F, C₁ and Br.

The term “heteroatom” includes oxygen (O), nitrogen (N), sulfur (S) and silicon (Si). In some embodiments, a heteroatom is selected from N and S. In some embodiments, the heteroatom is O.

Unless otherwise specified, the symbol “R” is a general abbreviation that represents a substituent group that is selected from acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound includes more than one R, R′, R″, R′″ and R″″ group, they are each independently selected.

For groups with solvent exchangeable protons, the ionized form is equally contemplated. For example, —COOH also refers to —COO— and —OH also refers to —O⁻.

Any of the compounds disclosed herein can be made into a pharmaceutically acceptable salt. The term “pharmaceutically acceptable salts” includes salts of compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts. The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides any of the compounds disclosed herein in a prodrug form. Prodrugs of the compounds described herein are those compounds that readily undergo chemical changes under physiological conditions to provide the compounds of the present invention.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate.

Illustrative esters, also referred to as active esters, include but are not limited to 1-indanyl, N oxysuccinimide; acyloxyalkyl groups such as acetoxymethyl, pivaloyloxymethyl, -acetoxyethyl, -pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, a-ethoxycarbonyloxyethyl, -ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆) cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted arylalkanoyl and optionally substituted heteroarylalkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. For example, the compounds may be labeled with deuterium (²H) or radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The symbol [symbol] , displayed perpendicular to a bond, indicates the point at which the displayed moiety is attached to the remainder of the molecule.

In some embodiments, the definition of terms used herein is according to IUPAC.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to the site of disease, injury, or defect, or to a particular organ or tissue system.

Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the patient being treated. It is further appreciated that local administration may be directly in the injury site, or locally in the surrounding tissue. Similar variations regarding local delivery to particular tissue types, such as organs, and the like, are also described herein. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

Depending upon the disease as described herein, the route of administration and/or whether the compounds and/or compositions are administered locally or systemically, a wide range of permissible dosages are contemplated herein, including doses falling in the range from about 1 g/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The effective use of the compounds, compositions, and methods described herein for treating or ameliorating diseases caused by pathogenic cells expressing PSMA may be based upon animal models, such as murine, canine, porcine, and non-human primate animal models of disease. For example, it is understood that prostate cancer in humans may be characterized by a loss of function, and/or the development of symptoms, each of which may be elicited in animals, such as mice, and other surrogate test animals. In particular, the mouse models described herein where cancer cells, such as LNCaP cells are subcutaneously implanted may be used to evaluate the compounds, the methods of treatment, and the pharmaceutical compositions described herein to determine the therapeutically effective amounts described herein.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21st ed., 2005)).

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

### 3. Compositions

The invention provides numerous chelators and metal ion complexes thereof. Generally, a chelator comprises a plurality of chelating agents that are linked together by way of two or more scaffold moieties. Chelating moieties bound together by two scaffold moieties such that at least one closed ring is formed can be referred to as closed chelators, macrocycles or macrocyclic chelators.

There are several factors to be considered in the design for an alpha chelating agent for anticancer therapy. Some of the key issues apart from the kinetics will be the high affinity for the target metal (such as Th) which at the same time needs to have a low exchange rate for other biologically significant metal ions. So, in our ligand design, the electronic properties of the target metal and ligand are considered and matched. The chelate should also be able to assume the appropriate coordination cavity size and geometry for the desired metal. In this case, Th, an actinide ion, is a “hard” cation and has a large charge-to-radius ratio. Hence, Th prefers “hard” electron donors and negatively charged oxygen donors. A coordination number of 8 or greater is generally preferred by actinide ions as they have a tendency to form stable complexes with ligands of high denticity; however, the selectivity towards the binding of the thorium will be determined by our design of the chelating unit. The effective but nonselective amino-carboxylic acid ligands such as DTPA can deplete essential biological metal ions from patients, thus causing serious health problems. Selecting the correct type of chelating unit, therefore, is an important factor in achieving high selectivity toward the specific metal ion.

A chelator can comprise numerous chelating moieties. Particularly useful chelators contain a number of chelating moieties sufficient to provide, for example, 6, 8 or 10 heteroatoms such as oxygen that coordinate with a metal ion to form a complex. The heteroatoms such as oxygen provide electron density for forming coordinate bonds with a positively charged ion, and such heteroatoms can thus be considered “donors”. In some embodiments, the plurality of chelating moieties of a chelator comprises a plurality of oxygen donors and a metal ion (such as a radionuclide) is chelated to the chelator via at least one of the oxygen donors. In some embodiments, a chelator comprises a plurality of oxygen donors and a metal ion (such as a radionuclide) is chelated to the chelator via a plurality or all of the oxygen donors.

### 3.1. Macrocycles

An exemplary embodiment provides a compound having the structure:

CH-L⁰-TA

where CH is a chelating agent comprising one or more chelating moieties capable of binding and complexing a metal ion.

In one aspect, the invention provides a macrocycle chelating agent of formula (M2+) or (M3+):

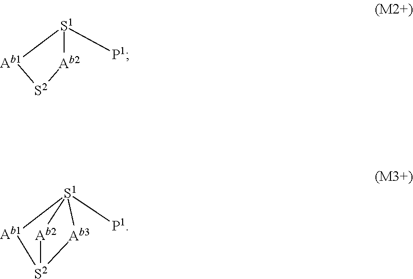

wherein S¹ and S² are independently selected scaffold moieties.
A¹, A², A³, and P¹ are independently selected chelating moieties.
Scaffold moieties and chelating moieties are as defined herein.

Any of the combinations of S¹, S², A¹, A², A³, and P¹ are encompassed by this disclosure and specifically provided by the invention.

In some embodiments, the macrocycle comprises a linker. In some embodiments, the linker is attached to a targeting moiety. In some embodiments, the macrocycle comprises a targeting moiety.

In some embodiments, the macrocycle comprises one or more additional, pendant chelating moieties (A^{x}), which may be attached to S¹, S², or P¹. Chelating moieties are as defined herein.

In some embodiments, the macrocycle comprises one, two or more modifying moieties. The modifying moieties can be the same or different.

### 2.1.1. Chelating Moieties

A¹, A², A³, and P¹ are chelating moieties having a structure independently selected from:

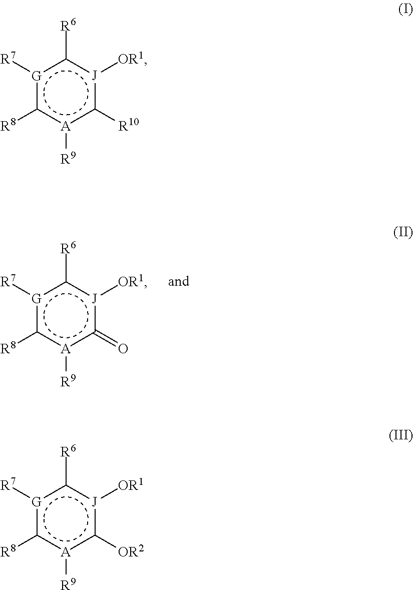

wherein
A and G are independently selected from carbon, nitrogen and oxygen;
wherein when A is oxygen, R⁹ is not present; and when G is oxygen, R⁷ is not present; J is selected from carbon and nitrogen;
each R¹ and R² is independently selected from H, an enzymatically labile group, a hydrolytically labile group, a metabolically labile group, a photolytically labile group and a single negative charge; each R⁶, R⁷, R⁸, R⁹, and R¹⁰ are independently selected from a bond to S¹ or S², alkanediyl attached to Si or S², H, substituted or unsubstituted alkyl, substituted or unsubstituted heteroalkyl, halogen, CN, —CF₃, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂,
wherein at least two of R⁶, R⁷, R⁸, R⁹, and R¹⁰ are optionally joined to form a ring system selected from substituted or unsubstituted cycloalkyl, substituted or unsubstituted heterocycloalkyl, substituted or unsubstituted aryl and substituted or unsubstituted heteroaryl; R¹⁷ and R¹⁸ are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R¹⁷ and R¹⁸, together with the atoms to which they are attached, are optionally joined to form a 5-, 6- or 7-membered ring; wherein A¹, A², and A³ are each attached to S¹ and S² through two members selected from R⁶, R⁷, R⁸, R⁹, and R¹⁰; and A¹ is attached to Si through a member selected from R⁶, R⁷, R⁸, R⁹, and R¹⁰.

In some embodiments, when any of A, A², and A³ has a structure according to formula (I), the respective chelating moiety is attached to S¹ and S² through R⁶ and R¹⁰.

In some embodiments, when any of A, A², and A³ has a structure according to formula (II) or (III), the respective chelating moiety is attached to S¹ and S² through R⁶ and R⁹.

In some embodiments, when P¹ has a structure according to formula (I), P¹ is attached to Si through R⁶ or R¹⁰.

In some embodiments, when P¹ has a structure according to formula (II) or (III), P¹ is attached to Si through R⁶ or R⁹.

In some embodiments, at least one of R⁶ and R¹⁰ in (I) is a bond attached to S¹ or S².

In some embodiments, A¹, A², A³, and P¹ are chelating moieties having a structure independently selected from:

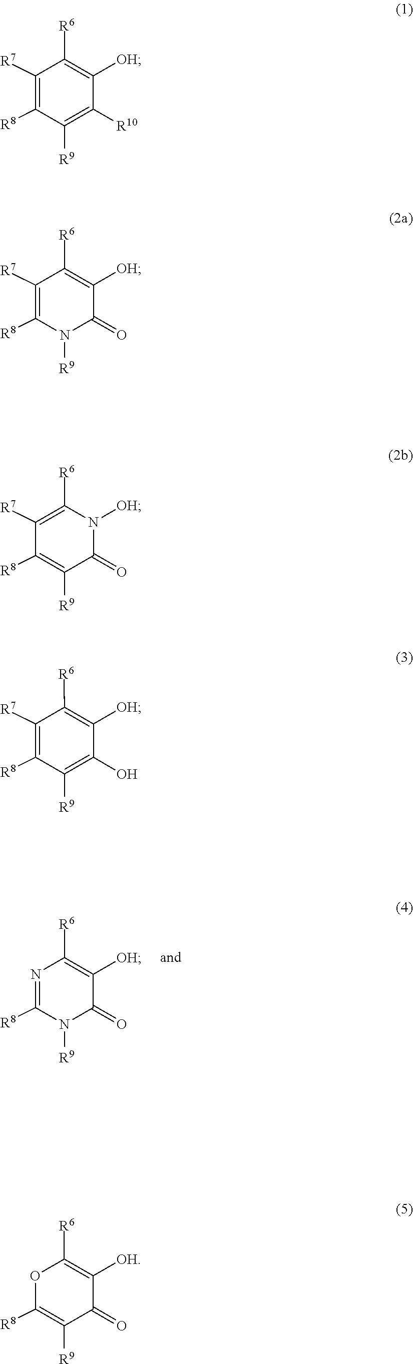

R⁶, R⁷, R⁸, R⁹, and R¹⁰ are as defined herein.

In some embodiments, A¹, A², A³, and P¹ are chelating moieties having a structure independently selected from:

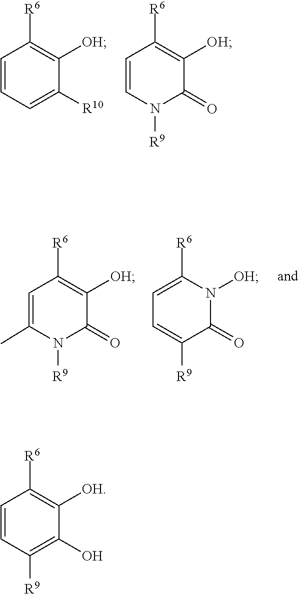

R⁶, R⁹, and R¹⁰ are as defined herein.

In some embodiments, A¹ and A² in formula (M2+) are the same. In some embodiments, A¹, A², and A³ in formula (M3+) are the same.

In some embodiments, at least one of A, A², and A³ does not have the structure:

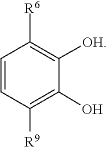

In some embodiments, A¹, A², and A³ do not have the structure:

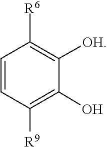

In some embodiments, P¹ comprises a linker. In some embodiments, the linker is attached to a targeting moiety. In some embodiments, P¹ comprises a targeting moiety.

In some embodiments, R⁶, R⁷, R⁸, R⁹, or R¹⁰ of A^{N} comprises a linker. In some embodiments, R⁶, R⁷, R⁸, R⁹, or R¹⁰ of P¹ is a linker.

In some embodiments, R⁶, R⁷, R⁸, R⁹, or R¹⁰ of P¹ is —C(O)NR¹⁷R¹⁸, wherein R¹⁷ or R¹⁸ comprises a linker. In some embodiments, R⁶, R⁷, R⁸, R⁹, or R¹⁰ of P¹ is —C(O)NR¹⁷R¹⁸, wherein R¹⁷ is H and R¹⁸ comprises a linker. In some embodiments, R⁶, R⁷, R⁸, R⁹, or R¹⁰ of P¹ is —C(O)NR¹⁷R¹⁸, wherein R¹⁷ is H and R¹⁸ is a linker.

In some embodiments, when P¹ has a structure according to formula (I) or (1), P¹ is attached to S¹ through R⁶, and R¹⁰ comprises a linker.

In some embodiments, when P¹ has a structure according to formula (I) or (1), P¹ is attached to S¹ through R¹⁰, and R⁶ comprises a linker.

In some embodiments, when P¹ has a structure according to formula (II), (III), (2a), (2b), (3), (4), or (5), P¹ is attached to S¹ through R⁶, and R⁹ comprises a linker.

In some embodiments, when P¹ has a structure according to formula (II), (III), (2a), (2b), (3), (4), or (5), P¹ is attached to S¹ through R⁹, and R⁶ comprises a linker. Linkers are as defined herein.

In some embodiments, P¹ comprises a modifying moiety. In some embodiments, R⁶, R⁷, R⁸, R⁹, or R¹⁰ of P¹ comprises a modifying moiety. In some embodiments, R⁶, R⁷, R⁸, R⁹, or R¹⁰ of P¹ is a modifying moiety.

In some embodiments, R⁶, R⁷, R⁸, R⁹, or R¹⁰ of P¹ is —C(O)NR¹⁷R¹⁸, wherein R¹⁷ or R¹⁸ comprises a modifying moiety. In some embodiments, R⁶, R⁷, R⁸, R⁹, or R¹⁰ of P¹ is —C(O)NR¹⁷R¹⁸, wherein R¹⁷ is H and R¹⁸ comprises a modifying moiety. In some embodiments, R⁶, R⁷, R⁸, R⁹, or R¹⁰ of P¹ is —C(O)NR¹⁷R¹⁸, wherein R¹⁷ is H and R¹⁸ is a modifying moiety.

In some embodiments, when P¹ has a structure according to formula (I) or (1), P¹ is attached to S¹ through R⁶, and R¹⁰ comprises a modifying moiety.

In some embodiments, when P¹ has a structure according to formula (I) or (1), P¹ is attached to S¹ through R¹⁰, and R⁶ comprises a modifying moiety.

In some embodiments, when P¹ has a structure according to formula (II), (III), (2a), (2b), (3), (4), or (5), P¹ is attached to S¹ through R⁶, and R⁹ comprises a modifying moiety.

In some embodiments, when P¹ has a structure according to formula (II), (III), (2a), (2b), (3), (4), or (5), P¹ is attached to S¹ through R⁹, and R⁶ comprises a modifying moiety.

Modifying moieties are as defined herein.

### 3.1.2. Scaffold Moieties

A “scaffold moiety” is any moiety useful for covalently linking two or more chelating moieties in any of the chelators (macrocycles) disclosed herein. In exemplary embodiments, any two scaffold moieties disclosed herein are joined via a plurality of chelating moieties to form a macrocycle. In exemplary embodiments, one or more scaffold moieties of a chelator is substituted with a linker. In one embodiment, the scaffold moiety is selected from substituted or unsubstituted alkyl and substituted or unsubstituted heteroalkyl. Exemplary scaffold moieties include linear or branched ethers and amines. In some embodiments, the linker is attached to a targeting moiety. In some embodiments, the scaffold moiety comprises a targeting moiety.

Other exemplary scaffold moieties include, but are not limited to:

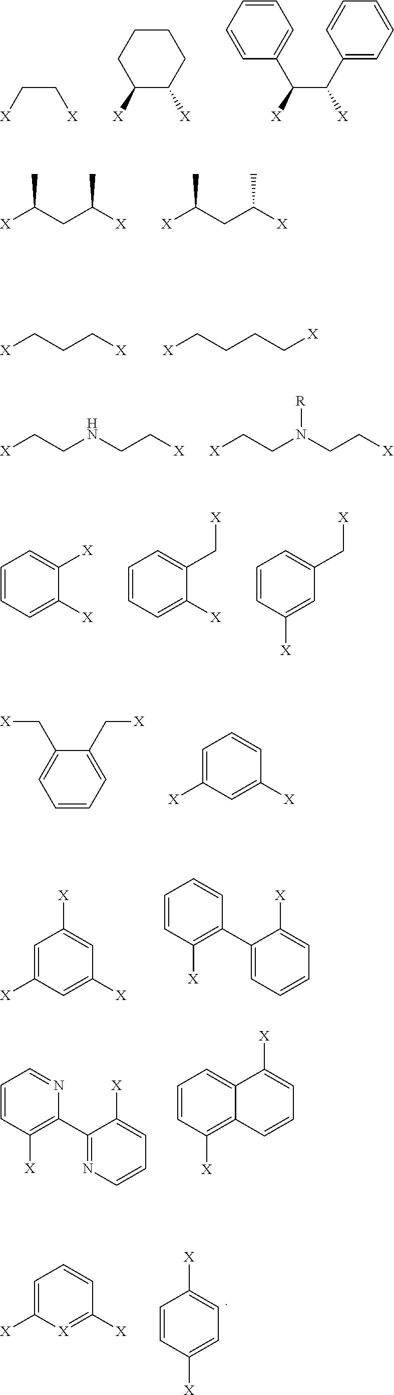

“X” represents a locus of attachment for a chelating moiety, and in exemplary embodiments includes a heteroatom such as nitrogen. Thus, in some embodiments, X is NR′R″, wherein R′ and R″ are independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, halogen, CN, CF₃, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R⁷, —C(O)NR¹⁷R¹⁸, —NR¹⁷C(O)R¹⁸, —NR¹⁷SO₂R¹⁸, —NO₂; and R¹⁷ and R¹⁸ are each independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; wherein at least one R′ or R″ comprises a bond to a chelating moiety. The chelating moiety can be attached to a scaffold via any appropriate linker.

In some embodiments, a scaffold moiety is linear. One exemplary scaffold moiety is X—(CH₂)₃—X—(CH₂)₄—X—(CH₂)₃—X, which is preferably substituted (e.g. with a linker) at at least one of the alkyl moieties. That is, one exemplary scaffold moiety is spermine based. Other exemplary scaffold moieties include

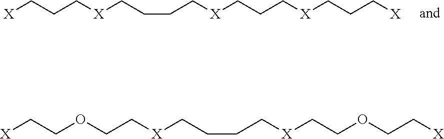

any of which is preferably substituted (e.g. with a linker) at at least one of the alkyl moieties. X is as given in the previous paragraph.

One preferred moiety for at least one of the X moieties is the 1,2-HOPO amide moiety, but those of skill in the art will appreciate that other chelating moieties in any used in any combination. In each of the scaffold structures, an aryl moiety or alkyl moiety can be substituted with one or more “aryl group substituent” or “alkyl group substituent” as defined herein.

A particularly useful scaffold moiety for any chelator described herein has the structure

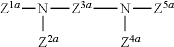

wherein Z^{1a}, Z^{2a}, Z^{3a}, Z^{4a} and Z^{5a} are selected from substituted or unsubstituted alkyl and substituted or unsubstituted heteroalkyl; and Z^{1a}, Z^{2a}, Z^{4a} and Z^{5a} comprise a bond to one of the chelating moieties.

In some embodiments, Z^{3a} is substituted or unsubstituted (C₁, C₂, C₃, C₄, C₅ or C₆) alkyl. In some embodiments, Z^{3a} is substituted or unsubstituted —(CH₂)ₘ(CH₂CH₂O)ₙ(CH₂)ₚ—, wherein m, n and p are integers independently selected from 1, 2, 3, 4, 5 and 6. In some embodiments, Z^{3a} is ethyl. In some embodiments, Z^{3a} is ethyl substituted with ═O.

In some embodiments, Z^{1a}, Z^{2a}, Z^{4a} and Z^{5a} have a structure selected from Z′R^{20a}N(H)C(O)Z″, Z′R^{20a}N(H)C(O)R^{21a}Z″ and Z′R^{21a}Z″ wherein Z′ is a bond to the second scaffold moiety, Z″ is a bond to one of the plurality of chelating moieties, R^{20a} is selected from substituted or unsubstituted alkyl and substituted or unsubstituted heteroalkyl. and R^{21a} is selected from substituted or unsubstituted alkyl and substituted or unsubstituted heteroalkyl. In some embodiments, R^{20a} is selected from substituted or unsubstituted (C₁, C₂, C₃, C₄, C₅ or C₆) alkyl and substituted or unsubstituted (C₁, C₂, C₃, C₄, C₅ or C₆) heteroalkyl. In some embodiments, R^{2′} is selected from substituted or unsubstituted ethyl. In some embodiments, R^{21a} is from substituted or unsubstituted —(CH₂)_{w}O— wherein w is selected from 1, 2, 3, 4, 5 and 6. In exemplary embodiments, w is 1 or 3.

In some embodiments, at least one of Z^{1a}, Z^{2a}, Z^{3a}, Z^{4a} and Z^{5a} is substituted with a linker.

Another particularly useful scaffold moiety for any chelator herein has the structure

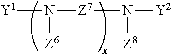

The index x is selected from 1, 2, 3 and 4. In exemplary embodiments, x is 1. In exemplary embodiments, x is 2. In exemplary embodiments, x is 3. In exemplary embodiments, x is 4.

Y¹ and Y² are each independently selected from H, substituted or unsubstituted alkyl and substituted or unsubstituted heteroalkyl. In exemplary embodiments, Y¹ and Y² are H.

Z⁷ is selected from substituted or unsubstituted alkyl and substituted or unsubstituted heteroalkyl. In exemplary embodiments, at least one Z⁷ is substituted with a linker. In some embodiments, each Z⁷ is independently substituted or unsubstituted (C₁, C₂, C₃, C₄, C₅ or C₆) alkyl. In exemplary embodiments, each Z⁷ is independently substituted or unsubstituted propyl or butyl. In some embodiments, each Z⁷ is independently substituted or unsubstituted heteroalkyl.

In exemplary embodiments, each Z⁷ is independently substituted or unsubstituted —(CH₂)ₘ(CH₂CH₂O)ₙ(CH₂)ₚ—, wherein m, n and p are integers independently selected from 1, 2, 3, 4, 5 and 6. In exemplary embodiments, each Z⁷ is substituted or unsubstituted —(CH₂)₂O(CH₂)₂—.

Z⁶ and Z⁸ are independently selected from —C(O)—, substituted or unsubstituted alkyl, and substituted or unsubstituted heteroalkyl; and each of Z⁶ and Z⁸ comprises a bond to one of the chelating moieties.

In exemplary embodiments, Z⁶ and Z⁸ are —C(O)—.

Another useful scaffold moiety has the structure:

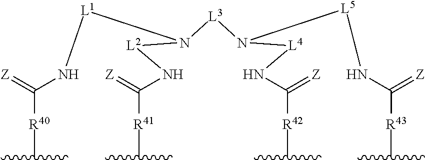

in which each Z is independently selected from O and S. In some embodiments, L³ comprises —(CH₂CH₂O)ₘR³¹— wherein m is an integer selected from 0, 1, 2, 3, 4, 5, 6, 7, 8 and 9. In some embodiments, m is 0. In some embodiments, m is 1. In some embodiments, L³ is —CH₂CH₂OCH₂CH₂—. L¹, L², L⁴, L⁵ and R³¹ are independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In exemplary embodiments, L¹, L², L⁴, L⁵ are independently selected substituted or unsubstituted (C₁, C₂, C₃, C₄, C₅ or C₆) alkyl. In some embodiments, R³¹ is substituted or unsubstituted (C₁, C₂, C₃, C₄, C₅ or C₆) alkyl. In exemplary embodiments, L¹, L², L⁴, L⁵ are independently selected substituted or unsubstituted ethyl. In some embodiments, R³¹ is substituted or unsubstituted ethyl. In exemplary embodiments, L¹, L², L⁴, L⁵ are ethyl, one or more of which is substituted with a linker. In some embodiments, L¹ is substituted with a linker. In some embodiments, L² is substituted with a linker. In some embodiments, L³ is substituted with a linker. In some embodiments, L⁴ is substituted with a linker. In some embodiments, L⁵ is substituted with a linker. In some embodiments, L¹ is ethyl substituted with a linker. In some embodiments, L² is ethyl substituted with a linker. In some embodiments, L³ is ethyl substituted with a linker. In some embodiments, L⁴ is ethyl substituted with a linker. In some embodiments, L⁵ is ethyl substituted with a linker. In some embodiments, R⁴⁰, R⁴¹, R⁴² and R⁴³ are bonds. In some embodiments, R⁴⁰, R⁴¹, R⁴² and R⁴³ are —(CH₂)_{w}O—, wherein w is selected from 0, 1, 2, 3, 4, 5, 6, 7, 8, 9 and 10. In exemplary embodiments, w is 3.

Another useful scaffold has the structure

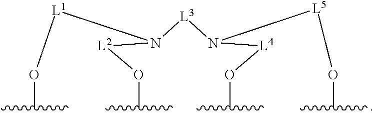

In some embodiments, L³ comprises —(CH₂CH₂O)ₘR³¹— wherein m is an integer selected from 0, 1, 2, 3, 4, 5, 6, 7, 8 and 9. In some embodiments, m is 0. In some embodiments, m is 1. In some embodiments, L³ is —CH₂CH₂OCH₂CH₂—. In some embodiments, L³ is —C(O)C(O)—. L¹, L², L⁴, L⁵ and R³¹ are independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In exemplary embodiments, L¹, L², L⁴, L⁵ are independently selected substituted or unsubstituted (C₁, C₂, C₃, C₄, C₅ or C₆) alkyl. In some embodiments, R³¹ is substituted or unsubstituted (C₁, C₂, C₃, C₄, C₅ or C₆) alkyl. In exemplary embodiments, L¹, L², L⁴, L⁵ are independently selected substituted or unsubstituted ethyl. In exemplary embodiments, L¹, L², L⁴, L⁵ are independently selected substituted or unsubstituted propyl. In some embodiments, R³¹ is substituted or unsubstituted ethyl. In exemplary embodiments, L¹, L², L⁴, L⁵ are ethyl, one or more of which is substituted with a linker. In some embodiments, L¹ is substituted with a linker. In some embodiments, L² is substituted with a linker. In some embodiments, L³ is substituted with a linker. In some embodiments, L⁴ is substituted with a linker. In some embodiments, L⁵ is substituted with a linker. In some embodiments, L¹ is propyl substituted with a linker. In some embodiments, L² is propyl substituted with a linker. In some embodiments, L³ is propyl substituted with a linker. In some embodiments, L⁴ is propyl substituted with a linker. In some embodiments, L⁵ is propyl substituted with a linker.

In some embodiments, a scaffold is selected from:

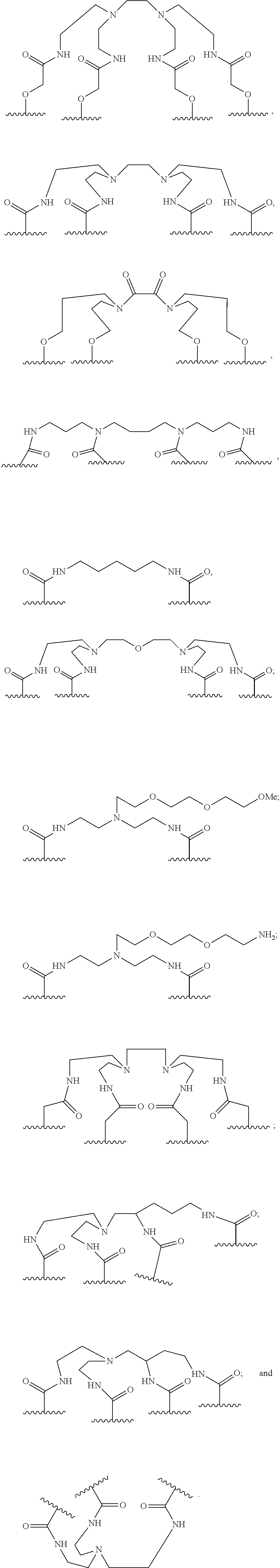

In any of these structures, one or more methyl, ethyl, propyl or butyl moieties can be substituted with one or more linkers. In some embodiments, two of these scaffold moieties, in which one or more methyl, ethyl, propyl or butyl moieties are optionally substituted with one or more linkers, are used to form a macrocycle.

In some embodiments, S¹, S², or both comprise a linker. In some embodiments, Si comprises a linker. In some embodiments, S² comprises a linker. In some embodiments, the linker is attached to a targeting moiety. In some embodiments, S¹, S², or both comprise a targeting moiety. In some embodiments, S¹ comprises a targeting moiety. In some embodiments, S² comprises a targeting moiety.

### 3.1.2a S¹

In some embodiments, S¹ has the structure:

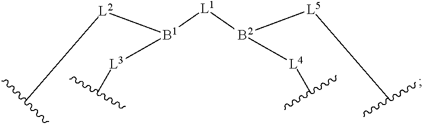

wherein L¹, L², L³, L⁴, and L⁵ are independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In some embodiments, L¹, L², L³, L⁴, and L⁵ are independently selected from substituted or unsubstituted alkyl and substituted or unsubstituted heteroalkyl.

In some embodiments, L¹, L², L³, L⁴, and L⁵ are independently selected from substituted or unsubstituted C₁-C₆ alkyl and substituted or unsubstituted C₁-C₆ heteroalkyl.

In some embodiments, P¹ is attached to L⁵, and L⁵ comprises a cleavable bond, allowing P¹ to be cleaved from the macrocycle under appropriate conditions (for instance, by an enzyme).

In some embodiments, the cleavable bond is an enzymatically cleavable bond, a hydrolytically cleavable bond, a metabolically cleavable bond, or a photolytically cleavable bond.

In some embodiments, the cleavable bond is part of a peptide, peptide mimetic, oligonucleotide, or DNA.

In some embodiments, one of L⁵ and L¹ is substituted with a linker. In some embodiments, L⁵ is substituted with a linker. Linkers are as defined herein.

B¹ and B² are independently selected from the elements capable of 3, 4, or 5 covalent bonds.

In some embodiments, B¹ and B² are independently selected from N, C, B, S¹, and P.

In some embodiments, B¹ and B² are independently selected from N and C.

In some embodiments, B¹ and B² are N.

In some embodiments, S¹ has the structure:

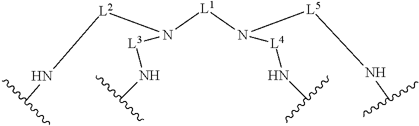

wherein L¹, L², L³, L⁴, and L⁵ are as defined herein.

In some embodiments, S¹ has the structure:

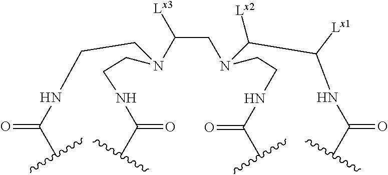

wherein L^{x1}, L^{x2} and L^{x3} are independently selected from H and a linker.

In some embodiments, only one of L^{x1}, L^{x2} and L^{x3} is a linker.

In some embodiments, L^{x1} is a linker. Linkers are as defined herein.

In some embodiments, L^{x1}, L^{x2} and L^{x3} are H.

In some embodiments, S¹ has the structure:

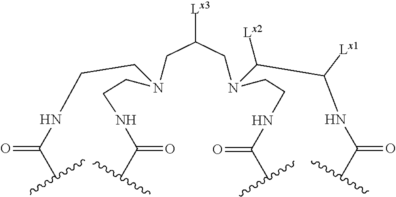

wherein L^{x1}, L^{x2} and L^{x3} are independently selected from H and a linker.

In some embodiments, only one of L^{x1}, L^{x2} and L^{x3} is a linker.

In some embodiments, L^{x3} is a linker. Linkers are as defined herein.

In some embodiments, L^{x1}, L^{x2} and L^{x3} are H.

In some embodiments, S¹ has the structure:

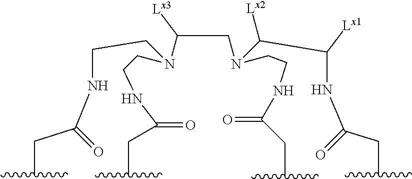

wherein L^{x1}, L^{x2} and L^{x3} are independently selected from H and a linker.

In some embodiments, only one of L^{x1}, L^{x2} and L^{x3} is a linker.

In some embodiments, L^{x1} is a linker. Linkers are as defined herein.

In some embodiments, L^{x1}, L^{x2} and L^{x3} are H.

In some embodiments, S¹ has the structure:

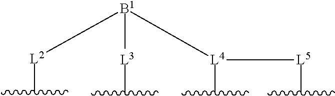

wherein B¹, L², L³, L⁴, and L⁵ are as defined herein.

In some embodiments, one of L², L³, L⁴, and L⁵ is substituted with a linker.

In some embodiments, L² is substituted with a linker.

In some embodiments, L⁵ is substituted with a linker. Linkers are as defined herein.

In some embodiments, S¹ has the structure:

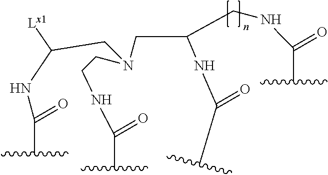

wherein n is 1, 2, 3, 4, 5, or 6; and L^{x1} is H or a linker.

In some embodiments, S¹ has the structure:

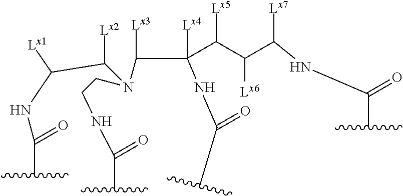

wherein L^{x1}, L^{x2}, L^{x3}, L^{x4}, L^{x5}, L^{x6}, and L^{x7} are independently selected from H and a linker.

In some embodiments, only one of L^{x1}, L^{x2}, L^{x3}, L^{x4}, L^{x5}, L^{x6}, and L^{x7} is a linker.

In some embodiments, L^{x1} is a linker. Linkers are as defined herein.

In some embodiments, L^{x1}, L^{x2}, L^{x3}, L^{x4}, L^{x5}, L^{x6}, and L^{x7} are H.

In some embodiments, S1 has the structure:

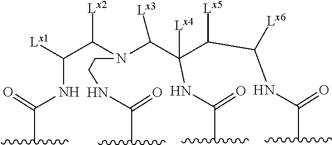

wherein L^{x1}, L^{x2}, L^{x3}, L⁴, L^{x5}, and L^{x6} are independently selected from H and a linker.

In some embodiments, only one of L^{x1}, L^{x2}, L^{x3}, L⁴, L^{x5}, and L^{x6} is a linker.

In some embodiments, L^{x1} is a linker. Linkers are as defined herein.

In some embodiments, L^{x1}, L^{x}, L^{x3}, L⁴, L^{x5}, and L^{x6} are H.

In some embodiments, S¹ has the structure:

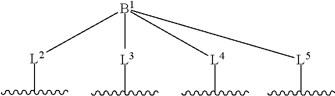

wherein B¹, L², L³, L⁴, and L⁵ are as defined herein.

In some embodiments, one of L², L³, L⁴, and L⁵ is substituted with a linker.

In some embodiments, L² is substituted with a linker.

In some embodiments, L⁵ is substituted with a linker. Linkers are as defined herein.

In some embodiments, S¹ has the structure:

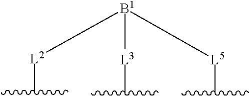

wherein B¹, L², L³, and L⁵ are as defined herein.

In some embodiments, one of L², L³, and L⁵ is substituted with a linker.

In some embodiments, L² is substituted with a linker.

In some embodiments, L⁵ is substituted with a linker. Linkers are as defined herein.

In some embodiments, S¹ has the structure:

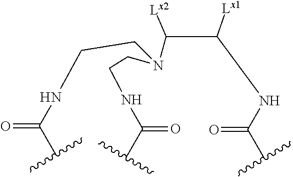

wherein L^{x1} and L^{x2} are independently selected from H and a linker.

In some embodiments, only one of L^{x1} and L^{x2} is a linker.

In some embodiments, L^{x1} is a linker. Linkers are as defined herein.

In some embodiments, L^{x1} and L^{x2} are H.

### 3.1.2b S²

In some embodiments, S² has the structure:

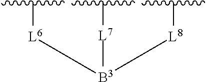

wherein L⁶, L⁷, and L⁸ are independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In some embodiments, L⁶, L⁷, and L⁸ are independently selected from substituted or unsubstituted alkyl and substituted or unsubstituted heteroalkyl.

In some embodiments, L⁶, L⁷, and L⁸ are independently selected from substituted or unsubstituted C₁-C₆ alkyl and substituted or unsubstituted C₁-C₆ heteroalkyl.

In some embodiments, one of L⁶, L⁷, and L⁸ is substituted with a linker. Linkers are as defined herein.

B³ is selected from the elements capable of 3, 4, or 5 covalent bonds.

In some embodiments, B³ is selected from N, C, B, S¹, and P.

In some embodiments, B³ is selected from N and C. In some embodiments, B³ is N.

In some embodiments, S² has the structure:

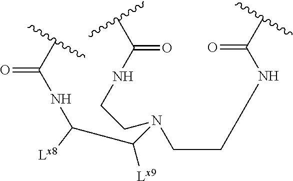

wherein L^{x8} and L^{x9} are independently selected from H and a linker.

In some embodiments, only one of L^{x8} and L^{x9} is a linker. In some embodiments, L^{x8} is a linker. Linkers are as defined herein.

In some embodiments, L^{x8} and L^{x9} are H.

In some embodiments, S² has the structure:

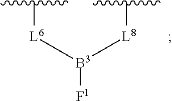

wherein L⁶ and L⁷ are independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In some embodiments, L⁶ and L⁷ are independently selected from substituted or unsubstituted alkyl and substituted or unsubstituted heteroalkyl.

In some embodiments, L⁶ and L⁷ are independently selected from substituted or unsubstituted C₁-C₆ alkyl and substituted or unsubstituted C₁-C₆ heteroalkyl.

In some embodiments, one of L⁶ and L⁷ is substituted with a linker. Linkers are as defined herein.

B³ is as defined herein.

F¹ is selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In some embodiments, F¹ is as defined herein.

In some embodiment S² has the structure:

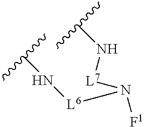

wherein L⁶, L⁷ and F¹ are as defined herein.

In some embodiment S² has the structure:

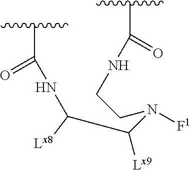

wherein L^{x8} and L^{x9} are independently selected from H and a linker.

In some embodiments, only one of L^{x8} and L^{x9} is a linker. In some embodiments, L^{x8} is a linker. Linkers are as defined herein.

In some embodiments, L^{x8} and L^{x9} are H.

F¹ is as defined herein.

In some embodiment S² has the structure:

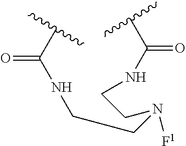

wherein F¹ is as defined herein.

In some embodiment S² has the structure:

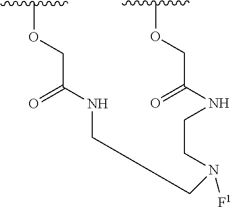

wherein F¹ is as defined herein.

In some embodiment S² has the structure:

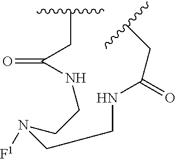

wherein F¹ is as defined herein.

### 3.1.3. Linker to Functional/Targeting Moiety

A “linker”, “linking member”, or “linking moiety” as used herein is a moiety that joins or potentially joins, covalently or noncovalently, a first moiety to a second moiety. In particular, a linker attaches or could potentially attach a chelator described herein to another molecule, such as a targeting moiety. In some embodiments, a linker attaches or could potentially attach a chelator described herein to a solid support. A linker comprising a reactive functional group that can be further reacted with a reactive functional group on a structure of interest in order to attach the structure of interest to the linker is referred to as a “functionalized linker”. In exemplary embodiments, a linker is a functionalized linker. In exemplary embodiments, a chelator comprises one or more functionalized linkers. In some embodiments, a linker comprises a targeting moiety. In some embodiments, a linker to a targeting moiety comprises a bond to the targeting moiety.

A linker can be any useful structure for that joins a chelator to a reactive functional group or a targeting moiety, such as an antibody. Examples of a linker include 0-order linkers (i.e., a bond), substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Further exemplary linkers include substituted or unsubstituted (C₁, C₂, C₃, C₄, C₅, C₆, C₇, C₈, C₉ or C₁₀) alkyl, substituted or unsubstituted heteroalkyl, —C(O)NR′—, —C(O)O—, —C(O)S—, and —C(O)CR′R″, wherein R′ and R″ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In some embodiments, a linker includes at least one heteroatom. Exemplary linkers also include —C(O)NH—, —C(O), —NH—, —S—, —O—, and the like. In an exemplary embodiment, a linker is a heteroalkyl substituted with a reactive functional group.

### 3.1.4 Reactive Functional Groups

In one embodiment, a linker comprises a reactive functional group (or a “reactive functional moiety”, used synonymously), which can be further reacted to covalently attach the linker to a targeting (or other) moiety. Reactive functional groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in March, Advanced Organic Chemistry (3rd Ed., John Wiley & Sons, New York, 1985); Hermanson, Bioconjugate Techniques (Academic Press, San Diego, 1996); and Feeney et al., Modification of Proteins, Advances in Chemistry Series, Vol. 198 (American Chemical Society, Washington, D.C., 1982).

In some embodiments, a reactive functional group refers to a group selected from olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds., Organic Functional Group Preparations, (Academic Press, San Diego, 1989)).

A reactive functional group can be chosen according to a selected reaction partner. As an example, an activated ester, such as an NHS ester will be useful to label a protein via lysine residues. Sulfhydryl reactive groups, such as maleimides can be used to label proteins via amino acid residues carrying an SH-group (e.g., cystein). Antibodies may be labeled by first oxidizing their carbohydrate moieties (e.g., with periodate) and reacting resulting aldehyde groups with a hydrazine containing ligand.

The reactive functional groups can be chosen such that they do not participate in, or interfere with, the reactions necessary to assemble the reactive ligand. Alternatively, a reactive functional group can be protected from participating in the reaction by means of a protecting group. Those of skill in the art understand how to protect a particular functional group so that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### 3.1.4a Amines and Amino-Reactive Groups

In one embodiment, a reactive functional group is selected from an amine, (such as a primary or secondary amine), hydrazine, hydrazide and sulfonylhydrazide. Amines can, for example, be acylated, alkylated or oxidized. Useful non-limiting examples of amino-reactive groups include N-hydroxysuccinimide (NHS) esters, sulfur-NHS esters, imidoesters, isocyanates, isothiocyanates, acylhalides, arylazides, p-nitrophenyl esters, aldehydes, sulfonyl chlorides, thiazolides and carboxyl groups.

NHS esters and sulfur-NHS esters react preferentially with a primary (including aromatic) amino groups of a reaction partner. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide.

Imidoesters are the most specific acylating reagents for reaction with amine groups of a molecule such as a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components (e.g., ε-amino group of lysine residues). Although unstable, Schiff bases are formed upon reaction of the protein amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry, 1981, 20: 4836-4842, e.g., teach how to modify a protein with carbodiimides.

### 3.1.4b Sulfhydryl and Sulfhydryl-Reactive Groups

In another embodiment, a reactive functional group is selected from a sulfhydryl group (which can be converted to disulfides) and sulfhydryl-reactive group. Useful non-limiting examples of sulfhydryl-reactive groups include maleimides, alkyl halides, acyl halides (including bromoacetamide or chloroacetamide), pyridyl disulfides, and thiophthalimides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

### 3.14c Other Reactive Functional Groups

Other exemplary reactive functional groups include:
(i) carboxyl groups and various derivatives thereof including, but not limited to, N-hydroxybenztriazole esters, acid halides, acyl imidazoles, thioesters, p-nitrophenyl esters, alkyl, alkenyl, alkynyl and aromatic esters;
(ii) hydroxyl groups, which can be converted to esters, ethers, aldehydes, etc.;
(iii) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(iv) dienophile groups, which are capable of participating in Diels-Alder reactions such as, for example, maleimido groups;
(v) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(vi) alkenes, which can undergo, for example, cycloadditions, acylation, Michael addition, etc; (vii) epoxides, which can react with, for example, amines and hydroxyl groups;
(ix) phosphoramidites and other standard functional groups useful in nucleic acid synthesis and
(x) any other functional group useful to form a covalent bond between the functionalized ligand and a molecular entity or a surface.
   3.1.4d Functional Groups with Non-specific Reactivities

In addition to the use of site-specific reactive moieties, the present invention contemplates the use of non-specific reactive groups to link a chelator to a targeting moiety. Non-specific groups include photoactivatable groups, for example.

Photoactivatable groups are ideally inert in the dark and are converted to reactive species in the presence of light. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

In exemplary embodiments, a linker joins a chelator to a targeting moiety. That is, in exemplary embodiments, a linker comprises a targeting moiety. In some embodiments, a chelator comprises a linker to a targeting moiety. Any linker described herein may be a linker comprising a reactive functional group that could react with a reactive functional group on a targeting moiety to join the linker to the targeting moiety. Any linker described herein may be a linker comprising a bond to a targeting moiety. The term “targeting moiety” refers to a moiety serves to target or direct the molecule to which it is attached (e.g., a chelator or a chelator complexed to a metal ion (such as a radionuclide)) to a particular location or molecule. Thus, for example, a targeting moiety may be used to target a molecule to a specific target protein or enzyme, or to a particular cellular location, to a particular cell type or to a diseased tissue. As will be appreciated by those in the art, the localization of proteins within a cell is a simple method for increasing effective concentration. For example, shuttling an imaging agent and/or therapeutic into the nucleus confines them to a smaller space thereby increasing concentration. Finally, the physiological target may simply be localized to a specific compartment, and the agents must be localized appropriately.

The targeting moiety can be a small molecule (e.g., MW<1000D), which includes both non-peptides and peptides. Examples of a targeting moiety also include peptides, polypeptides (including proteins, and in particular antibodies, which includes antibody fragments), nucleic acids, oligonucleotides, carbohydrates, lipids, hormones (including proteinaceous and steroid hormones (for instance, estradiol)), growth factors, lectins, receptors, receptor ligands, cofactors and the like. Targets of a targeting moiety can include a cell-surface receptor, complementary nucleic acid, a receptor, an antibody, an antigen or a lectin, for example. In various embodiments, a targeting or modifying moiety binds to albumin or other plasma protein to extend the half life of the compound of the invention. In various embodiments, the modifying or targeting moiety is a steroid hormone. In various embodiments, the modifying or targeting moiety is an aryl moiety, e.g., a haloaryl, e.g., iodophenyl, iodobenzyl, etc.

The targeting or modifying moiety can also serve as a pharmacokinetic modifying agent (circulating half-life, solubility, immune system activater, etc.).

In various embodiments, a targeting or modifying agent is conjugated through a branch point in L^{o} or TA. In a preferred embodiment, L^{o} is a branched linker and one branch comprises a modifying moiety and another branch comprises TA.

In exemplary embodiments, a targeting moiety can bind to a target with high binding affinity.

In other words, a targeting moiety with high binding affinity to a target has a high specificity for or specifically binds to the target. In some embodiments, a high binding affinity is given by a dissociation constant K_{d} of about 10⁻⁷ M or less. In exemplary embodiments, a high binding affinity is given by a dissociation constant K_{d} of about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less or about 10⁻¹⁵ M or less. A compound may have a high binding affinity for a target if the compound comprises a portion, such as a targeting moiety, that has a high binding affinity for the target.

In exemplary embodiments, a targeting moiety is an antibody. An “antibody” refers to a protein comprising one or more polypeptides substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

While a targeting moiety may be appended to a chelator in order to localize the compound to a specific region in an animal, certain chelators have a natural affinity for cells, tissue, organs or some other part of the animal. For example, a chelator disclosed herein might have a natural or intrinsic affinity for bone. Thus, in some embodiments, a chelator (macrocycle), does not comprise a targeting moiety or a linker to a targeting moiety. A chelator lacking a targeting moiety can be used in any method that does not require specific targeting.

In some embodiments, a chelator comprises a linker to a solid support. That is, any linker described herein may be a linker comprising a reactive functional group that could react with a reactive functional group on a solid support to join the linker to the solid support. Any linker described herein may be a linker comprising a bond to a solid support. A “solid support” is any material that can be modified to contain discrete individual sites suitable for the attachment or association of a chelator. Suitable substrates include biodegradable beads, non-biodegradable beads, silica beads, magnetic beads, latex beads, glass beads, quartz beads, metal beads, gold beads, mica beads, plastic beads, ceramic beads, or combinations thereof. Of particular use are biocompatible polymers, including biodegradable polymers that are slowly removed from the system by enzymatic degradation. Example biodegradable materials include starch, cross-linked starch, poly(ethylene glycol), polyvinylpyrrolidine, polylactides (PLA), polyglycolides (PGA), poly(lactide-co-glycolides) (PLGA), polyanhydrides, polyorthoesters, poly(DTH iminocarbonate), poly(bisphenol A iminocarbonate), polycyanoacrylate, polyphosphazene, mixtures thereof and combinations thereof. Other suitable substances for forming the particles exist and can be used. In some embodiments, a solid support is a bead comprising a cross-linked starch, for example, cross-linked potato starch. Beads made from starch are completely biodegradable in the body, typically by serum amylase, a naturally occurring enzyme found in the body. In these embodiments, the chelator optionally further comprises a targeting moiety or a linker to a targeting moeity. In cases where a chelator that is attached to a solid support does not comprise a targeting moiety, the chealtor can be localized directly by the practitioner, for example, by direct surgical implantation.

In some embodiments, a linker has the structure -L¹¹-X, wherein L¹¹ is selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and X is a reactive functional group or a targeting moiety.

In some embodiments, L¹¹ is selected from substituted or unsubstituted alkyl and substituted or unsubstituted heteroalkyl. In some embodiments, L¹¹ is heteroalkyl. In some embodiments, L¹¹ is (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ or C₂₀) alkyl in which 1, 2 or 3 atoms are replaced with a heteroatom, such as nitrogen or oxygen.

In some embodiments, X is selected from —NH₂ and —CO(O)H.

In some embodiments, -L¹¹-X is selected from

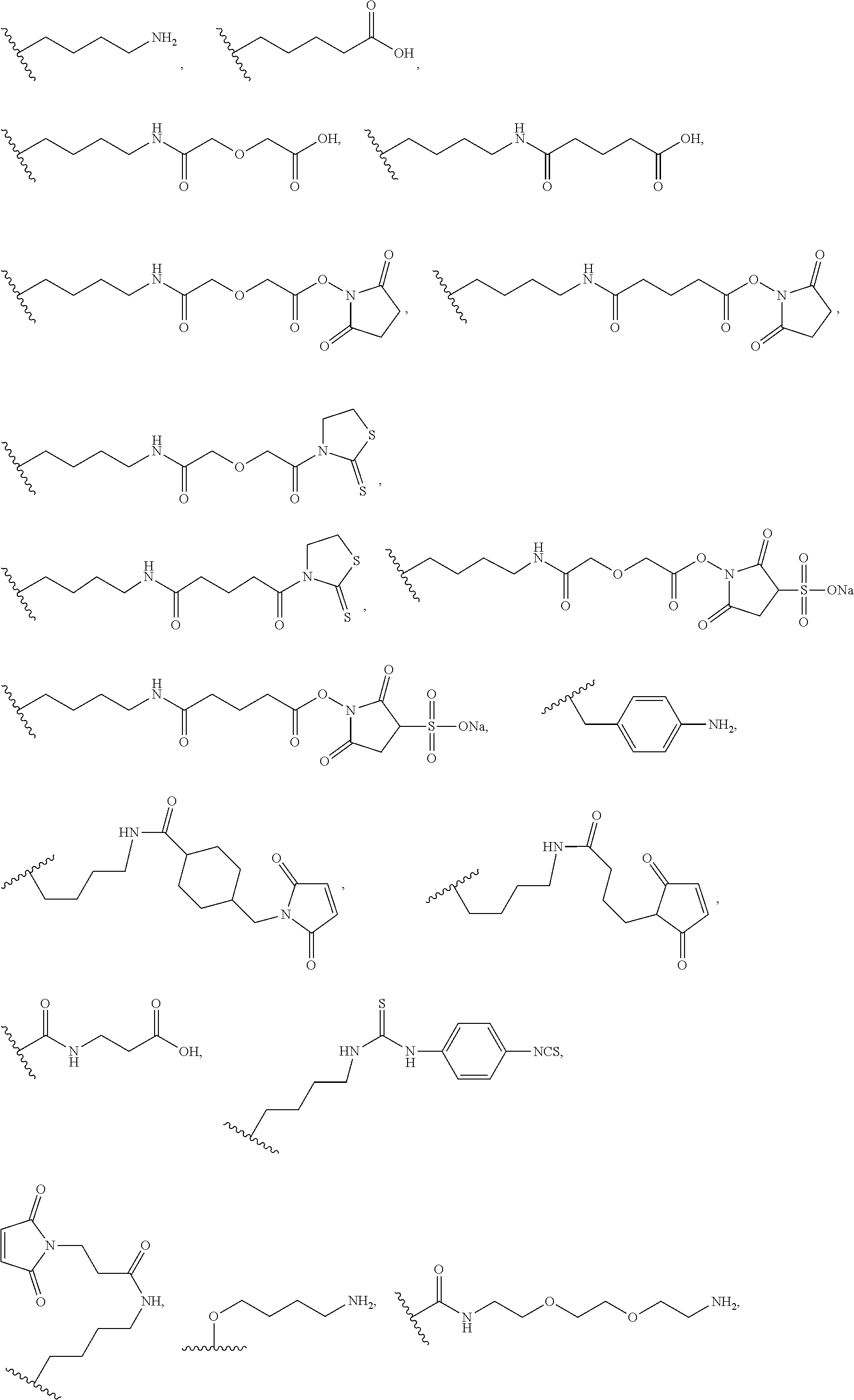

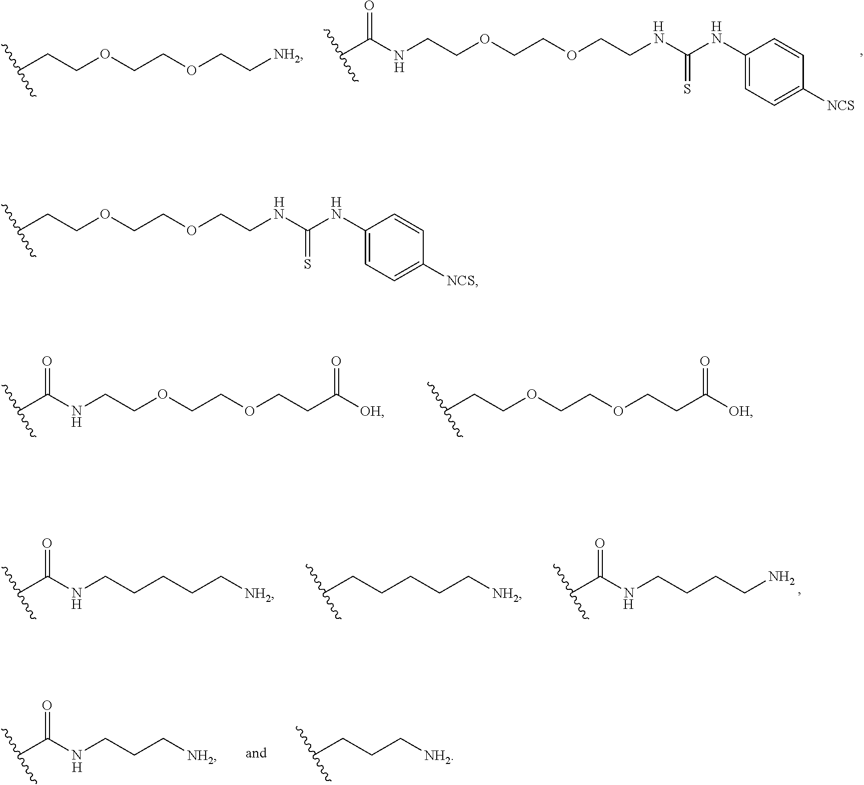

In exemplary embodiments, X is a targeting moiety.

In exemplary embodiments, a linker is a linker to a targeting moiety. In some embodiments, the targeting moiety is selected from a polypeptide, a nucleic acid, a lipid, a polysaccharide, a small molecule, a cofactor and a hormone. In exemplary embodiments, the targeting moiety is an antibody or antibody fragment.

In some embodiments, a linker includes an aliphatic carbon chain or a poly-ethyleneglycol (PEG) chain. Thus, a linker can comprise a structure selected from:

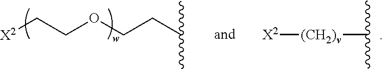

The integer v is selected from 1 to 20, and w is an integer from 1 to 1,000 or 1 to 500 or 1 to 100 or 1 to 50 or 1 to 10.

Exemplary X² groups include OH, alkoxy, and one of the following structures:

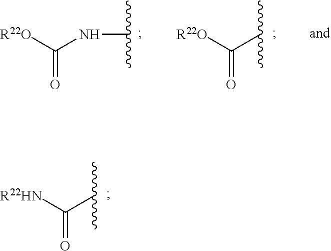

wherein R²² is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl. The integer v is selected from 1 to 20, and w is an integer from 1 to 1,000 or 1 to 500 or 1 to 100 or 1 to 50 or 1 to 10.

In some embodiments, a linker has the structure:

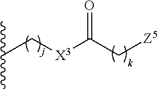

wherein Z⁵ is selected from H, OR²³, SR²³, NHR²³, OCOR²⁴, OC(O)NHR²⁴, NHC(O)OR²³, OS(O)₂OR²³, and C(O)R²⁴. R²³ is selected from H, substituted or unsubstituted alkyl, and substituted or unsubstituted heteroalkyl. R²⁴ is selected from H, OR²⁵, NR²⁵NH₂, SH, C(O)R²⁵, NR²⁵H, substituted or unsubstituted alkyl and substituted or unsubstituted heteroalkyl. R²⁵ is selected from H, substituted or unsubstituted alkyl and substituted or unsubstituted alkyl. X³ is selected from O, S and NR²⁶, wherein R²⁶ is a member selected from H, substituted or unsubstituted alkyl and substituted or unsubstituted heteroalkyl. The integers j and k are members independently selected from 1, 2, 3, 4, 5, 6, 7, 8, 9, 10, 11, 12, 13, 14, 15, 16, 17, 18, 19 and 20. In some embodiments, the integers j and k are members independently selected from 1, 2, 3, 4, 5, 6.

In a linker with multiple reactive functional groups, a particular functional group can be chosen such that it does not participate in, or interfere with, the reaction controlling the attachment of the functionalized spacer component to another ligand component. Alternatively, the reactive functional group can be protected from participating in the reaction by the presence of a protecting group. Those of skill in the art understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

### 2.1.4. Modifying Moiety

In some embodiments, one, two or all of S¹, S² and P¹ comprise a modifying moiety. Each of the modifying moieties can be the same or different. The modifying moiety modifies various properties of the macrocycle and/or a complex formed between the macrocycle and a metal ion, such as solubility, charge, or affinity. In some embodiments, the modifying moiety does not interact with the metal when the macrocycle is complexed to a metal. In some embodiments, the modifying moiety is a solubilizing group, a hormone-derived moiety, a prodrug moiety (for example, with a cleavable moiety), an oligonucleotide, ssDNA, dsDNA, RNA, or a peptide. The solubilizing group improves solubility of the macrocycle and/or a complex formed between the macrocycle and a metal ion in aqueous media. In some embodiments, the hormone (of the homone-derived moiety) is a steroid. In some embodiments, the steroid is estradiol. In some embodiments, the modifying moiety is an estradiol-derived moiety.

Peptides of a hydrophilic and hydrophobic nature by virtue of their amino acid composition may be used to tune solubility of the macrocycle and/or a complex formed between the macrocycle and a metal ion.

In some embodiments, S² comprises a modifying moiety. In some embodiments, P¹ comprises a linker; and S¹, S², or both comprise a modifying moiety. In some embodiments, S¹ comprises a linker; and S², Pi, or both comprise a modifying moiety. In some embodiments, S¹ comprises a linker; and Pi comprises a modifying moiety.

In some embodiments, F¹ comprises a modifying moiety. In some embodiments, F¹ is a modifying moiety.

In some embodiments, F¹ is substituted or unsubstituted heteroalkyl. In some embodiments, F¹ is a substituted or unsubstituted polyether. In some embodiments, F¹ comprises a hormone or hormone analog, e.g., estradiol of an estradiol-derived moiety. In some embodiments, F¹ is a polyether substituted with a hormone or hormone analog, e.g., estradiol or an estradiol-derived moiety.

In some embodiments, F¹ is selected from:

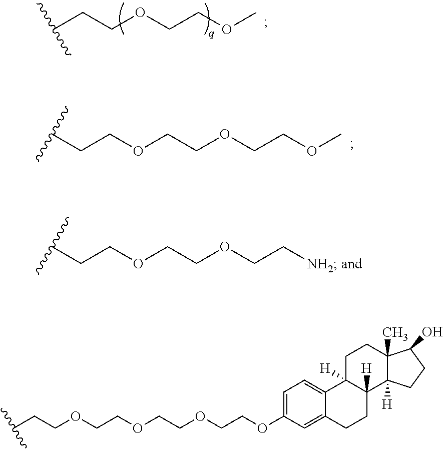

in which the index q is an integer from 0 to 500, e.g., 2 to 250.

In some embodiments, F¹ is a peptide. In some embodiments, F¹ is

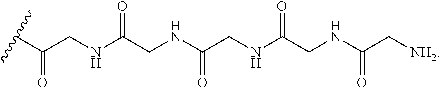 [SMILES: *C(=O)CNC(=O)CNC(=O)CNC(=O)CNC(=O)CN]

In some embodiments, F¹ comprises an oligunucleotide.

In some embodiments, F¹ is a linker.

In various embodiments, a targeting or modifying moiety binds to albumin or other plasma protein to extend the half life of the compound of the invention. In various embodiments, the modifying or targeting moiety is a steroid hormone. In various embodiments, the modifying or targeting moiety is an aryl moiety, e.g., a haloaryl, e.g., iodophenyl, iodobenzyl, etc. The targeting or modifying moiety can also serve as a pharmacokinetic modifying agent (circulating half-life, solubility, immune system activater, etc.).

### 3.1.5 Targeting Moiety

The compounds of the invention include at least one targeting moiety capable of binding to a cell-surface receptor, e.g., PSMA, LHIRH and/or the somatostatin receptor. Ligands binding to PSMA, LHRH and somatostatin receptor are known in the art, and methods of functionalizing these ligands such that they are appropriate conjugation partners for agents of use in diagnosing, imaging and/or treating diseases such as primary and metastatic prostate cancer lesions. Non-limiting reference to useful targeting agents, methods of exploring structural diversity in PSMA and other ligands, and methods of forming and using conjugates of these ligands are set forth in, for example, Anderson et al., Bioorg Med Chem. 2007; 15(21): 6678-6686, which discloses various peptidomimetic ligands. See, also, Benesova et al., J Nucl Med. 2015; 56:914-920; Anderson et al., Theranostics 2017; 7(7): 1928-1939, which discusses various phosphoramidate ligands. Such ligands are also the focus of, for example, Ganguly et al., Nucl Med Biol 2015; 42(10): 780-787, which discloses a phopsphoramidate peptide-mimetic; Schally et al., Eur J Endocrin. 1999; 141: 11-14; and Bouvet et al., EJNMMI Res. 2016; 6: 40, disclosing small molecule PSMA inhibitors which are radiolabeled. Wu et al., Biorg Med Chem 2007; 15(231): 7434-7443 provides PSMA inhibitor analogs of the natural substrate retaining elements of this substrate, ie, folyl-γ-Glu. This structure includes a serine residue at the P1 position, which provides a convenient locus for attachment of a phosphoramidate moiety, which can bind zinc used by PSMA. Other disclosures on PSMA inhibitors include Umbrecht et al., EJNMI Res 2017: 7-9; and Goumi et al., Molecules 2017; 22: 525.

It is within the skills of the worker ordinarily skilled in the art to understand these references and the larger body of learning from which they are taken, to judiciously select an appropropriate PSMA or LHRH ligand and prepare a derivated for coupling to the chelating agent (CH) via linker L^{o} to prepare the compositions of the invention.

Furthermore, these cited references, and others readily available and understandable to those of ordinary skill in the art, include descriptions of the in vitro and in vivo characterization of diagnostic, imaging or therapeutic conjugates of the ligand conjugates. The described procedures are readily modified as needed and reproduced by the ordinarily skilled worker.

In an exemplary embodiment, the precursor for the targeting agent, the conjugate binding partner has the formula:

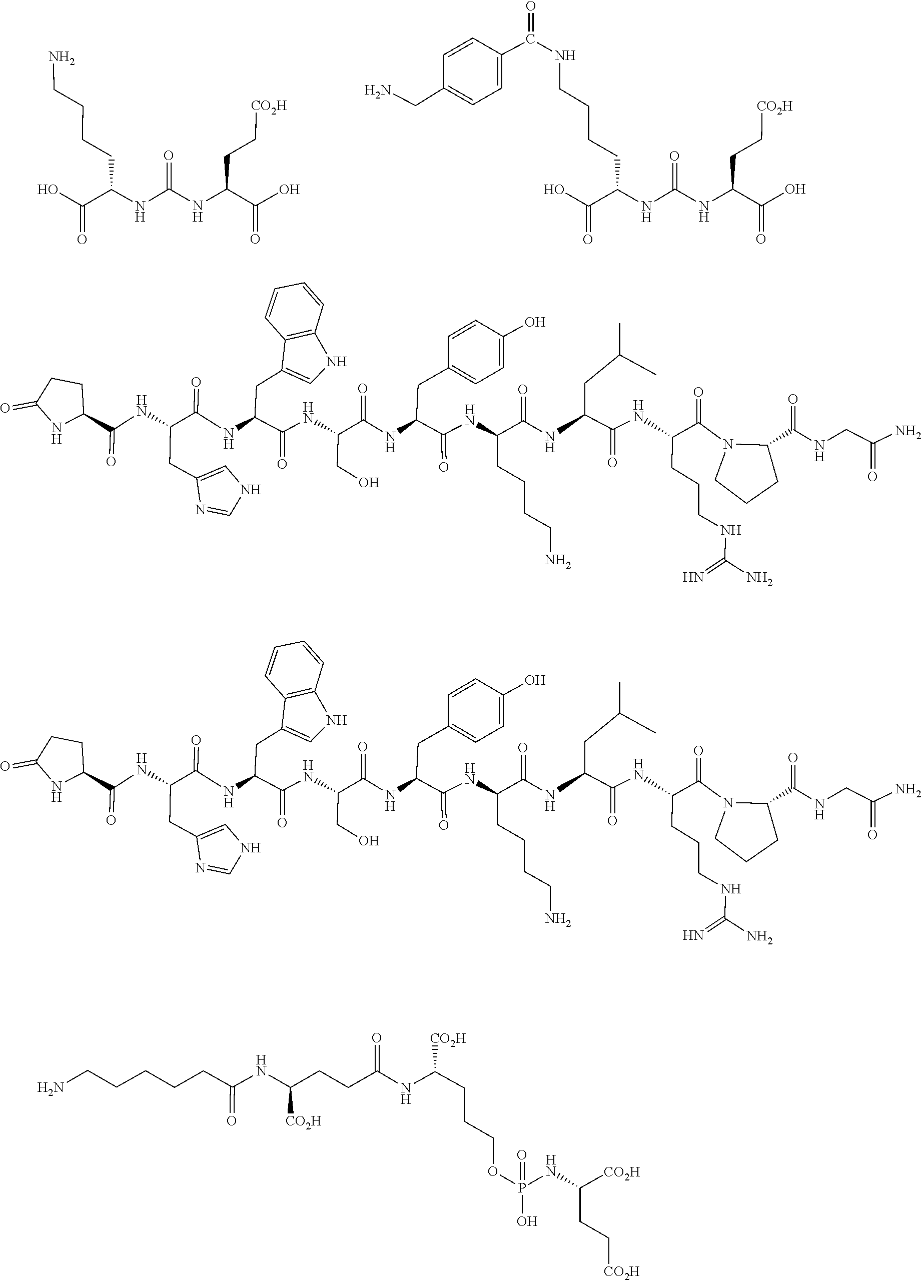

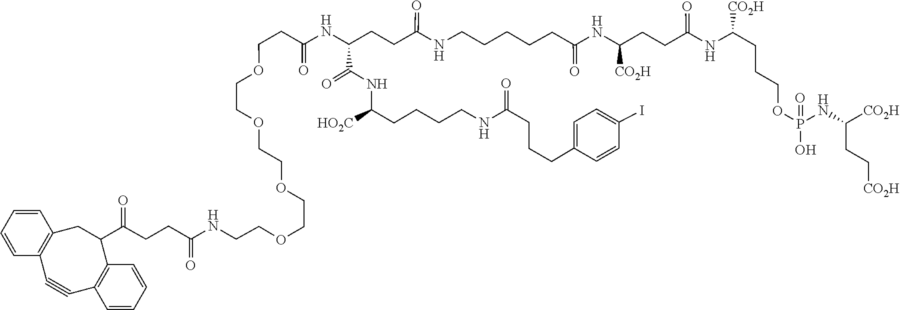 [SMILES: O=C(O)CC[C@H](NP(=O)(O)OCCC[C@H](NC(=O)CC[C@H](NC(=O)CCCCCNC(=O)CC[C@@H](NC(=O)CCOCCOCCOCCOCCNC(=O)CCC(=O)C1Cc2ccccc2C#Cc2ccccc21)C(=O)N[C@@H](CCCCNC(=O)CCCc1ccc(I)cc1)C(=O)O)C(=O)O)C(=O)O)C(=O)O]

### 3.1.6 Exemplary Macrocycles

In some embodiments, the invention provides a macrocycle having a structure selected from:

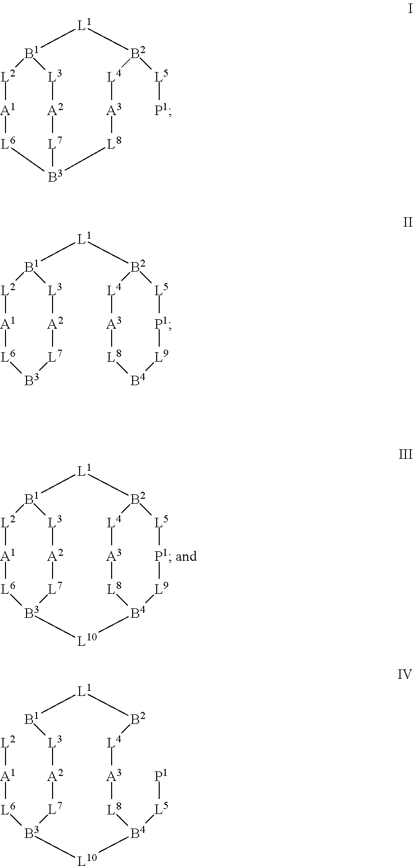

wherein B¹, B², and B³ are independently selected from N and C; L¹, L², L³, L⁴, L⁵, L⁶, L⁷, and L⁸ are independently selected from substituted or unsubstituted alkyl, and substituted or unsubstituted heteroalkyl; A¹, A², and A³ are members independently selected from:

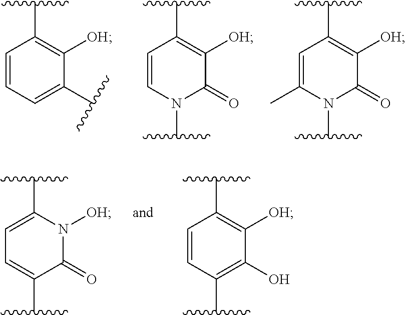

and P¹ is a member selected from:

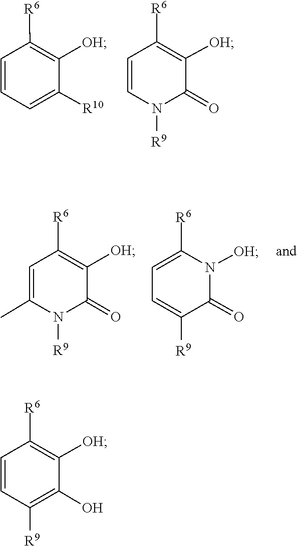

wherein R⁶, R⁹, and R¹⁰ are as defined herein, with the proviso that R⁶, R⁹, or R¹⁰ is a bond to L⁵.

In some embodiments, the macrocycle is covalently modified with at least one linker. In various embodiments, the linker is conjugated to a targeting agent.

In some embodiments, one of L¹, L², L³, L⁴, L⁵, L⁶, L⁷, L⁸, and P¹ is substituted with a linker.

In various embodiments, the linker is conjugated to a targeting agent.

In some embodiments, P¹ is substituted with a linker. In various embodiments, the linker is conjugated to a targeting agent.

In some embodiments, L⁵ is substituted with a linker. In various embodiments, the linker is conjugated to a targeting agent.

In some embodiments, L² is substituted with a linker. In various embodiments, the linker is conjugated to a targeting agent.

Additional relevant disclosure is found in WO 2018/045385, the entire disclosure of which is incorporated herein by reference in its entirety for all purposes. This disclosure finds particular relevance with respect to Formula III and IV.

In some embodiments, the invention provides a macrocycle having the structure:

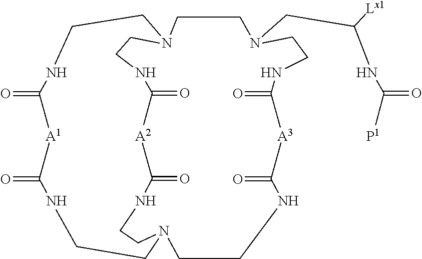

wherein A¹, A², and A³ are members independently selected from:

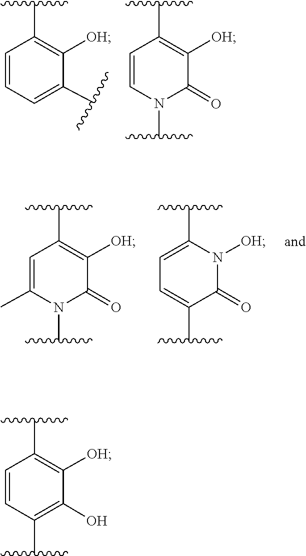

and
P¹ is a member selected from:

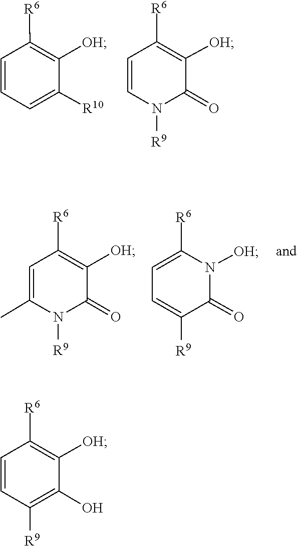

wherein R⁶, R⁹, and R¹⁰ are as defined herein, with the proviso that R⁶, R⁹, or R¹⁰ is a bond to L⁵; and L^{x1} is H or a linker.

In some embodiments, the macrocycle is covalently modified with at least one linker. In various embodiments, the linker is conjugated to a targeting agent.

In some embodiments, L^{x1} is a linker or P¹ is substituted with a linker. In various embodiments, the linker is conjugated to a targeting agent.

In some embodiments, P¹ is substituted with a linker. In various embodiments, the linker is conjugated to a targeting agent.

In some embodiments, L^{x1} is a linker.

In some embodiments, the invention provides a macrocycle having the structure:

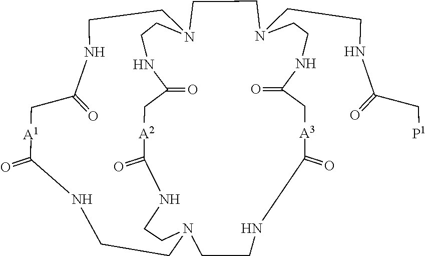

wherein A¹, A², and A³ are members independently selected from:

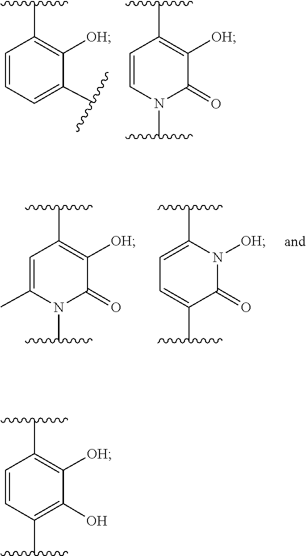

P¹ is a member selected from:

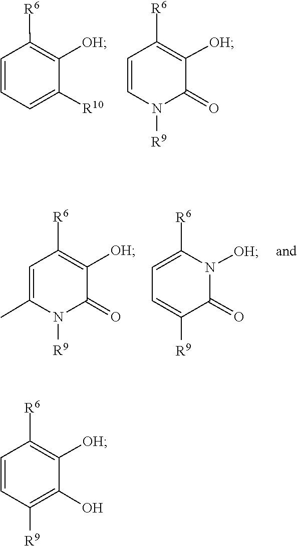

wherein R⁶, R⁹, and R¹⁰ are as defined herein, with the proviso that R⁶, R⁹, or R¹⁰ is a bond to L⁵.

In some embodiments, the macrocycle is covalently modified with at least one linker. In various embodiments, the linker is conjugated to a targeting agent.

In some embodiments, P¹ is substituted with a linker. In various embodiments, the linker is conjugated to a targeting agent.

In some embodiments, the invention provides a macrocycle having the structure:

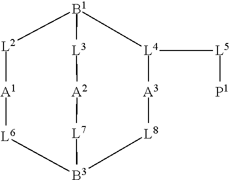

wherein B¹ and B³ are independently selected from N and C; L², L³, L⁴, L⁵, L⁶, L⁷, and L⁸ are independently selected from substituted or unsubstituted alkyl, and substituted or unsubstituted heteroalkyl; A¹, A², and A³ are members independently selected from:

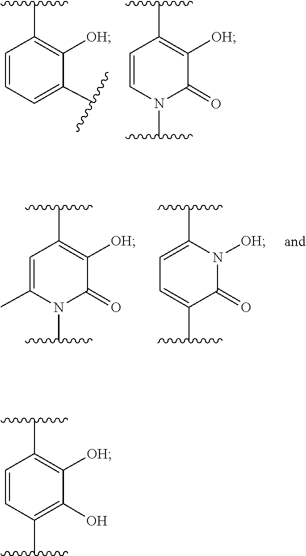

and
P¹ is a member selected from:

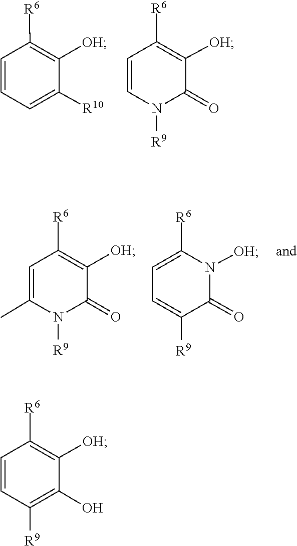

wherein R⁶, R⁹, and R¹⁰ are as defined herein, with the proviso that R⁶, R⁹, or R¹⁰ is a bond to L⁵.

In some embodiments, the macrocycle is covalently modified with at least one linker. In various embodiments, the linker is conjugated to a targeting agent.

In some embodiments, one of L², L³, L⁴, L⁵, L⁶, L⁷, L⁸, and P¹ is substituted with a linker. In various embodiments, the linker is conjugated to a targeting agent.

In some embodiments, P¹ is substituted with a linker. In various embodiments, the linker is conjugated to a targeting agent.

In some embodiments, L⁵ is substituted with a linker. In various embodiments, the linker is conjugated to a targeting agent.

In some embodiments, L² is substituted with a linker. In various embodiments, the linker is conjugated to a targeting agent.

In some embodiments, the invention provides a macrocycle having the structure:

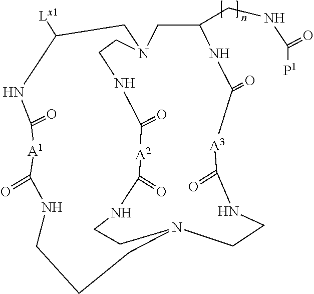

wherein n is 1, 2, 3, 4, 5, or 6; A¹, A², and A³ are members independently selected from:

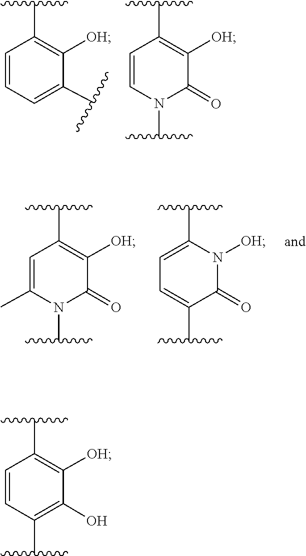

and
P¹ is a member selected from:

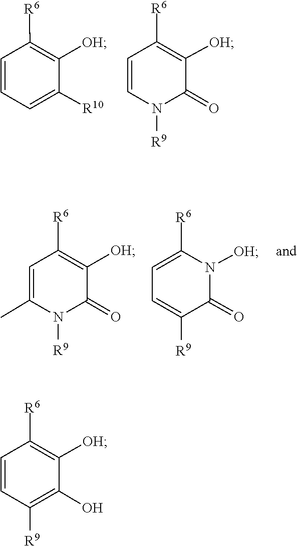

wherein R⁶, R⁹, and R¹⁰ are as defined herein, with the proviso that R⁶, R⁹, or R¹⁰ is a bond to L⁵; and L^{x1} is H or a linker.

In some embodiments, the macrocycle is covalently modified with at least one linker. In various embodiments, the linker is conjugated to a targeting agent.

In some embodiments, L^{x1} is a linker or P¹ is substituted with a linker. In various embodiments, the linker is conjugated to a targeting agent.

In some embodiments, P¹ is substituted with a linker. In various embodiments, the linker is conjugated to a targeting agent.

In some embodiments, L^{x1} is a linker.

In some embodiments, the invention provides a macrocycle having the structure:

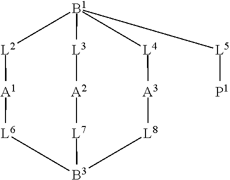

wherein B¹ is C; B³ is N or C; L², L³, L⁴, L⁵, L⁶, L⁷, and L⁸ are independently selected from substituted or unsubstituted alkyl, and substituted or unsubstituted heteroalkyl; A¹, A², and A³ are members independently selected from:

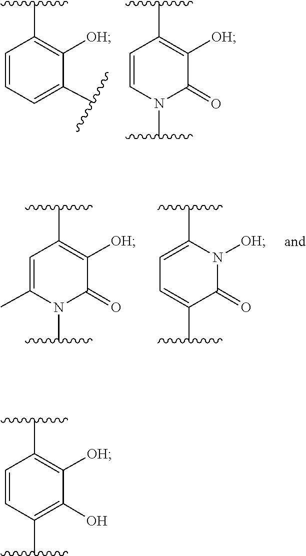

and
A^{p1} is a member selected from:

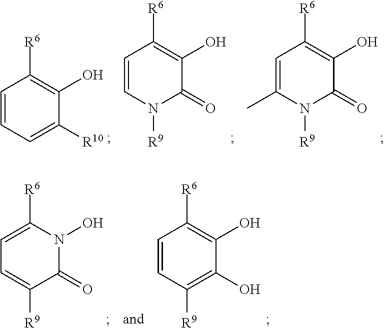

wherein R⁶, R⁹, and R¹⁰ are as defined herein, with the proviso that R⁶, R⁹, or R¹⁰ is a bond to L⁵.

In some embodiments, the macrocycle is covalently modified with at least one linker. In various embodiments, the linker is conjugated to a targeting agent.

In some embodiments, one of L², L³, L⁴, L⁵, L⁶, L⁷, L⁸, and P¹ is substituted with a linker. In various embodiments, the linker is conjugated to a targeting agent.

In some embodiments, P¹ is substituted with a linker. In various embodiments, the linker is conjugated to a targeting agent.

In some embodiments, L⁵ is substituted with a linker. In various embodiments, the linker is conjugated to a targeting agent.

In some embodiments, L² is substituted with a linker. In various embodiments, the linker is conjugated to a targeting agent.

In some embodiments, the invention provides a macrocycle having the structure:

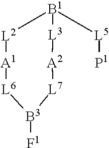

wherein B¹ and B³ are independently selected from N and C; F¹ is selected from H, substituted or unsubstituted alkyl, substituted or unsubstituted heteroalkyl, substituted or unsubstituted aryl, substituted or unsubstituted heteroaryl and substituted or unsubstituted heterocycloalkyl; L², L³, L⁵, L⁶, and L⁷ are independently selected from substituted or unsubstituted alkyl, and substituted or unsubstituted heteroalkyl; A¹ and A² are members independently selected from:

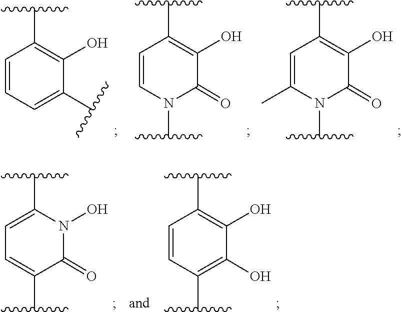

and

A^{p1} is a member selected from:

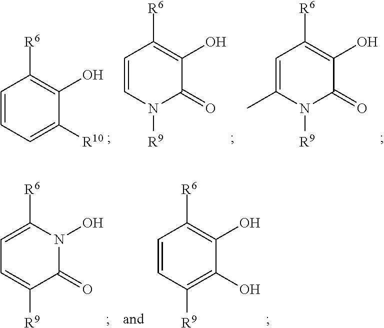

wherein R⁶, R⁹, and R¹⁰ are as defined herein, with the proviso that R⁶, R⁹, or R¹⁰ is a bond to L⁵.

In some embodiments, the macrocycle is covalently modified with at least one linker. In various embodiments, the linker is conjugated to a targeting agent.

In some embodiments, one of L², L³, L⁵, L⁶, L⁷, and A^{p1} is substituted with a linker. In various embodiments, the linker is conjugated to a targeting agent.

In some embodiments, P¹ is substituted with a linker. In various embodiments, the linker is conjugated to a targeting agent.

In some embodiments, L⁵ is substituted with a linker. In various embodiments, the linker is conjugated to a targeting agent.

In some embodiments, L² is substituted with a linker. In various embodiments, the linker is conjugated to a targeting agent.

In some embodiments, F¹ is modifying moiety. Modifying moieties are as defined herein.

In some embodiments, the invention provides a macrocycle having the structure:

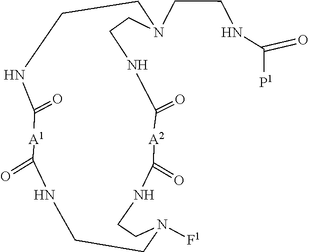

wherein F¹ is selected from H, substituted or unsubstituted alkyl, substituted or unsubstituted heteroalkyl, substituted or unsubstituted aryl, substituted or unsubstituted heteroaryl and substituted or unsubstituted heterocycloalkyl; A¹ and A² are members independently selected from:

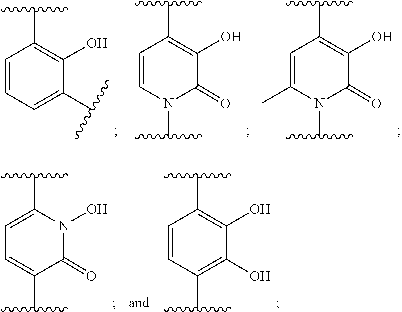

P¹ is a member selected from:

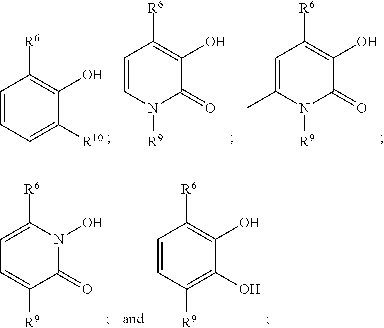

wherein R⁶, R⁹, and R¹⁰ are as defined herein, with the proviso that R⁶, R⁹, or R¹⁰ is a bond to L⁵.

In some embodiments, the macrocycle is covalently modified with at least one linker.

In some embodiments, P¹ is substituted with a linker. In various embodiments, the linker is conjugated to a targeting agent.

In some embodiments, F¹ is modifying moiety. Modifying moieties are as defined herein.

In an exemplary embodiment, the invention provides a macrocycle macrocycle conjugate having the formula:

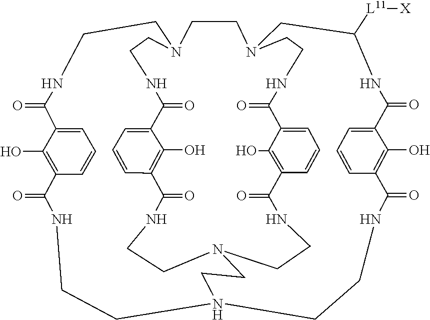

In an exemplary embodiment, the invention provides a macrocycle conjugate having the formula:

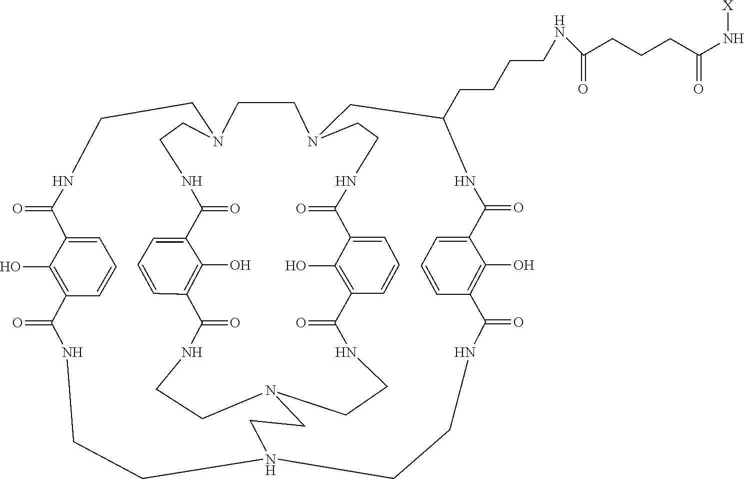

Additional exemplary macrocycles are shown in the Examples.

### 3.2. Complexes

In one aspect, the invention provides a complex of a macrocycle disclosed herein with a metal ion.

### Complexes

In one aspect, the invention provides a complex of a compound (ligand) disclosed herein with a metal ion.

In an exemplary embodiment, the complex is a complex of a +2 metal cation, e.g., Ca⁺² or Mg⁺². In a preferred embodiment, the +2 metal cation protects a reactive moiety on a reagent contacting the complex from unproductive reaction with the complex during the course of modification of the complex with the reagent. An exemplary reactive moiety is an imide moiety, e.g, N-hydoxysuccinimide (NHS). In a preferred embodiment, the complex of the +2 metal cation is converted to an NHS ester by contacting the complex with a reagent comprising a NHS moiety. In a preferred embodiment, the +2 metal cation is displaced following reaction with the reagent, e.g., following NHS ester formation, by a cation of valence higher than +2, e.g., +3 or +4. In a preferred embodiment, the cation of higher valence is selected from an ion of lanthanides, transition metals and actinides.

In some embodiments, the complex is luminescent.

In some embodiments, the complex includes a metal ion that is a known radioisotope.

In another aspect, the invention provides a complex of a compound (ligand) disclosed herein with an element, or ion thereof, from periods 4, 5, 6 and 7 and/or from groups 13, 14, 15, 16. In another aspect, the invention provides a complex of a compound (ligand) disclosed herein with an element, or ion thereof, from periods 3, 4, 7, 8, 9, 10, 11, 13, 14, and 15. In some embodiments, the invention provides a complex of a compound (ligand) disclosed herein with an element, or ion thereof, from periods 3, 4, and 13.

In some embodiments, complexes disclosed in WO 2013/187971 A2 are excluded.

Any of the combinations of compounds (ligands) disclosed herein and a metal ion disclosed herein are encompassed by this disclosure and specifically provided by the invention.

Exemplary complexes are shown in the Examples.

Any of the combinations of macrocycles disclosed herein and a metal ion disclosed herein are encompassed by this disclosure and specifically provided by the invention.

In some embodiments, the complex is luminescent. 3.2.1. Metals

In some embodiments, the metal is an actinide. In some embodiments, the actinide is thorium (Th).

In some embodiments, the metal is a lanthanide. In some embodiments, the lanthanide is terbium (Tb). In some embodiments, the lanthanide is europium (Eu). In some embodiments, the lanthanide is dysprosium (Dy). In some embodiments, the lanthanide is lutetium (Lu). In some embodiments, the lanthanide is gadolinium (Gd).

In some embodiments the metal is yttrium (Y). In some embodiments, the metal is zirconium (Zr).

In some embodiments, the metal ion is yttrium(III). In some embodiments, the metal ion is europium(III).

In some embodiments, the metal ion is terbium(III).

In some embodiments, the metal ion is zirconium(IV). In some embodiments, the metal ion is thorium(IV).

In some embodiments, the metal (ion) is a radionuclide. In some embodiments, the metal ion is ²²⁷Th(IV).

In some embodiments, the metal ion is ⁸⁹Zr(IV).

In some embodiments, the metal is ¹⁷⁷Lu.

In some embodiments, the metal is ¹⁶⁶Ho.

In some embodiments, the metal is ¹⁵³Sm.

In some embodiments, the metal is ⁹⁰Y.

In some embodiments, the metal is ⁸⁶Y.

In some embodiments, the metal is ¹⁶⁶Dy.

In some embodiments, the metal is ¹⁶⁵Dy.

In some embodiments, the metal is ¹⁶⁹Er.

In some embodiments, the metal is ¹⁷Yb

In some embodiments, the metal is ²²⁵Ac.

In some embodiments, the metal is ¹⁴⁹Tb.

In some embodiments, the metal is ¹⁵³Gd.

In some embodiments, the metal is ²³⁰U.

In some embodiments, the metal is ¹¹¹In.

In some embodiments, the metal is ⁶⁷Ga.

In some embodiments, the metal is ⁶⁷Cu.

In some embodiments, the metal is ⁶⁴Cu.

In some embodiments, the metal is ¹⁸⁶Re.

In some embodiments, the metal is ¹⁸⁸Re.

In some embodiments, the metal is ¹¹¹Ag.

In some embodiments, the metal is ¹⁰⁹Pd.

In some embodiments, the metal is ²¹²Pb.

In some embodiments, the metal is ²⁰³Pb.

In some embodiments, the metal is ²¹²Bi. In some embodiments, the metal is ²¹³Bi.

In some embodiments, the metal is ^{195m}Pt.

In some embodiments, the metal is ²⁰¹Tl. In some embodiments, the metal is ⁵⁵Co.

In some embodiments, the metal is ^{99m}Tc.

### 3.2.1.1. Radionuclides

The chelating moieties disclosed herein can be used to bind metal ions, in particular, a radionuclide. The term “radionuclide” or “radioisotope” refers to a radioactive isotope or element with an unstable nucleus that tends to undergo radioactive decay. Numerous decay modes are known in the art and include alpha decay, proton emission, neutron emission, double proton emission, spontaneous fission, cluster decay, β⁻ decay, positron emission (β⁺ decay), electron capture, bound state beta decay, double beta decay, double electron capture, electron capture with positron emission, double positron emission, isomeric transition and internal conversion.

Exemplary radionuclides include alpha-emitters, which emit alpha particles during decay. In some embodiments, a radionuclide is an emitter of a gamma ray or a particle selected from an alpha particle, an electron and a positron.

In some embodiments, the radionuclide is an actinide. In some embodiments, the radionuclide is a lanthanide. In some embodiments, the radionuclide is a 3⁺ ion. In some embodiments, the radionuclide is a 4⁺ ion. In some embodiments the radionuclide is a 2⁺ ion.

Of particular use in the complexes provided herein are radionuclides selected from isotopes of U, Pu, Fe, Cu, Sm, Gd, Tb, Dy, Ho, Er, Yb, Lu, Y, Th, Zr, In, Ga, Bi, Ra, At and Ac. In some embodiments, a radionuclide is selected form radium-223, thorium-227, astatine-211, bismuth-213, Lutetium-177, and actinium-225. Other useful radioisotopes include bismuth-212, iodine-123, copper-64, iridium-192, osmium-194, rhodium-105, samarium-153, and yttrium-88, yttrium-90, and yttrium-91. In exemplary embodiments, the radionuclide is thorium, particularly selected from thorium-227 and thorium-232. In some embodiments, thorium-226 is excluded. In some embodiments, U is excluded. In some embodiments, uranium-230 is excluded. That is, in some embodiments, a radionuclide is not U, or a radionuclide is not uranium-230 or a radionuclide is not thorium-226.

²³²Th exists in nature as an α-emitter with a half life of 1.4×10¹⁰ yr. In aqueous solution, Th(IV) is the only oxidation state. Thorium(IV) ion is bigger than Pu(IV) and usually forms complexes with 9 or higher coordination number. For example, the crystal structure of both Th(IV) complexes of simple bidentate 1,2-HOPO and Me-3,2-HOPO have been determined as nine coordinated species.

Similar to other actinide ions, thorium(IV) prefers forming complexes with oxygen, especially negative oxygen donor ligands. Thorium(IV) also prefers octadentate or higher multidentate ligands:

| Ligand | Acac | NTA | HEDTA* | EDTA** | DTPA | TTHA |
|---|---|---|---|---|---|---|
| Ligand Type | Bi-dentate | Tetra- | Hexa- | Hexa- | Octa- | Deca- |
| Log K₁ | 7.85 | 16.9 | 18.5 | 25.3 | 30.34 | 31.9 |

*with one alcoholic oxygen and three carboxyl groups;
**with four carboxyl groups.

Other radionuclides with diagnostic and therapeutic value that can be used with the compounds disclosed herein can be found, for example, in U.S. Pat. Nos. 5,482,698 and 5,601,800; and Boswell and Brechbiel, Nuclear Medicine and Biology, 2007 October, 34(7): 757-778 and the manuscript thereof made available in PMC 2008 Oct. 1.

### 4. Uses

The chelators and complexes disclosed herein can be used in a wide variety of therapeutic and diagnostic settings.

In another embodiment, the compounds described herein can be internalized into the targeted pathogenic cells by binding to PSMA. In particular, PSMA selectively and/or specifically binds the conjugate, and internalization can occur, for example, through PSMA mediated endocytosis. Once internalized, conjugates containing a releasable linker can complete delivery of the drug to the interior of the target cell. Without being bound by theory, it is believed herein that in those cases where the drug is toxic to normal cells or tissues, such delivery system can decrease toxicity against those non-target cells and tissues because the releasable linker remains substantially or completely intact until the compounds described herein are delivered to the target cells. Accordingly, the compounds described herein act intracellularly by delivering the drug to an intracellular biochemical process, which in tum decreases the amount of unconjugated drug exposure to the host animal's healthy cells and tissues.

The conjugates described herein can be used for both human clinical medicine and veterinary applications. Thus, the host animal harboring the population of pathogenic cells and treated with the compounds described herein can be human or, in the case of veterinary applications, can be a laboratory, agricultural, domestic, or wild animal. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity.

The drug delivery conjugate compounds described herein can be administered in a combination therapy with any other known drug whether or not the additional drug is targeted.

Illustrative additional drugs include, but are not limited to, peptides, oligopeptides, retro-inverso oligopeptides, proteins, protein analogs in which at least one non-peptide linkage replaces apeptide linkage, apoproteins, glycoproteins, enzymes, coenzymes, enzyme inhibitors, amino acids and their derivatives, receptors and other membrane proteins, antigens and antibodies thereto, haptens and antibodies thereto, hormones, lipids, phospholipids, liposomes, toxins, antibiotics, analgesics, bronchodilators, beta-blockers, antimicrobial agents, antihypertensive agents, cardiovascular agents including antiarrhythmics, cardiac glycosides, antianginals, vasodilators, central nervous system agents including stimulants, psychotropics, antimanics, and depressants, antiviral agents, antihistamines, cancer drugs including chemotherapeutic agents, tranquilizers, anti-depressants, H-2 antagonists, anticonvulsants, antinauseants, prostaglandins and prostaglandin analogs, muscle relaxants, anti-inflammatory substances, stimulants, decongestants, antiemetics, diuretics, antispasmodics, antiasthmatics, anti-Parkinson agents, expectorants, cough suppressants, mucolytics, and mineral and nutritional additives.

In one aspect, the invention provides a method of treating a disease in an animal comprising administering a complex disclosed herein to the animal, whereby the disease is ameliorated or eliminated.

In one aspect, the invention provides a method of diagnosing a disease in an animal comprising (a) administering a complex disclosed herein to the animal and (b) detecting the presence or absence of a signal emitted by the complex. In some embodiments, the detecting step comprises obtaining an image based on the signal.

In some embodiments, the disease is cancer. In various embodiments, the cancer is selected from primary or metastatic prostate cancer lesions.

In some embodiments, the complex comprises a linker to a targeting moiety and the method further comprises localizing the complex to a targeting site in the animal by binding the targeting moiety to the targeting site.

The compounds disclosed herein are particularly well suited for the preparation of stable, pre-labeled antibodies for use in the diagnosis and treatment of cancer and other diseases. For example, antibodies expressing affinity for specific tumors or tumor-associated antigens are labeled with a diagnostic radionuclide-complexed chelate, and the labeled antibodies can be further stabilized through lyophilization. Where a chelate is used, it generally is covalently attached to the antibody. The antibodies used can be polyclonal or monoclonal, and the radionuclide-labeled antibodies can be prepared according to methods known in the art. The method of preparation will depend upon the type of radionuclide and antibody used. A stable, lyophilized, radiolabeled antibody can be reconstituted with suitable diluent at the time of intended use, thus greatly simplifying the on site preparation process. The methods of the invention can be applied to stabilize many types of pre-labeled antibodies, including, but not limited to, polyclonal and monoclonal antibodies to tumors associated with melanoma, colon cancer, breast cancer, prostate cancer, etc. Such antibodies are known in the art and are readily available.

In some embodiments, cleavage of P¹ from the macrocycle (for example, when L⁵ comprises a cleavable bond as disclosed herein) results in a detectable change in a property (such as MRI signal or fluorescence) of the macrocycle or complex thereof. This mechanism can be used, for example, to detect an enzyme capable of cleaving an enzymatically cleavable bond of L⁵.

The chelators and complexes of the invention are also of use in in vitro applications such as drug discovery, e.g., in vitro screening. An exemplary compound of the invention is utilized in a luminescence mode, e.g., TRF or TR-FRET. The compounds of the invention provide a rare instance in which a probe compound is the same as the therapeutic compound. This is also true of the use of a compound of the invention as a theranostic, e.g, where the compound is dual labeled with more than one metal ion, e.g., Th and Y or Th and Ar. The compound can be imaged in real time after administration to trace the delivery and/or distribution of the therapeutic compound

### 5. Synthesis

Any scaffold moiety can be derivatized with at least one linker, such as a functionalized linker. Thus, in one exemplary embodiment, a linker, such as a functionalized linker, can be attached to the scaffold moiety. In another exemplary embodiment, a linker, such as a functionalized linker, is attached to a chelating moiety. A functionalized linker can reacted to form a bond with a targeting moiety. The linker can also be attached to any other linker within a compound.

Scaffold moieties that include a linker can be prepared by the following exemplary methods.

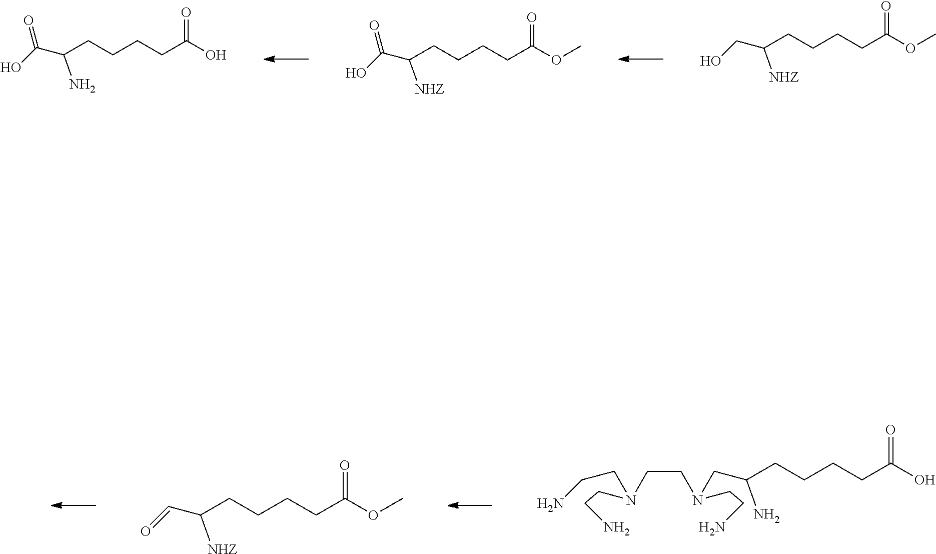

Scheme 1.1. Reverse synthetic scheme for carboxyl functionalized H22 cap-amine.

Other functionalize scaffolds include those in which the chiral carbon is placed on the central ethylene bridge of H22-amine. An exemplary route to such a scaffold initiates with 2,3-Diaminopropionic acid, as its carboxyl group is connected directly to the amine backbone to give a very rigid geometry, extended carboxyl chain is needed to provide flexibility for eventual protein conjugating. A synthetic scheme to the scaffold is shown in scheme 1.2.

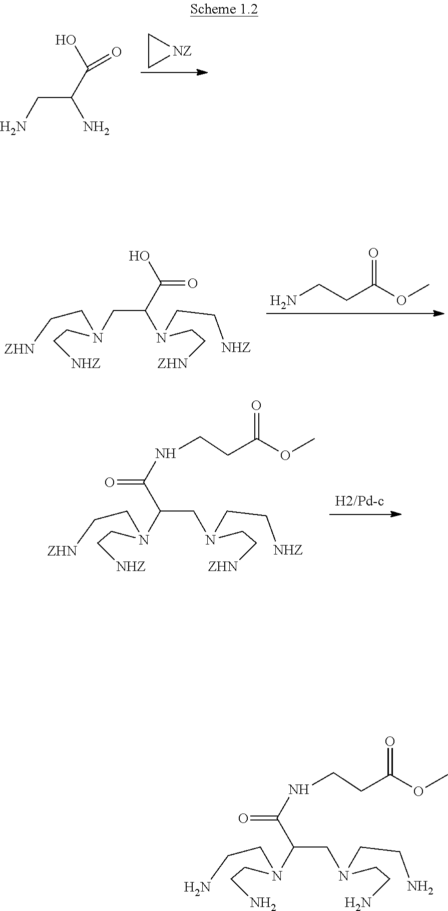

Variations on this synthesis include the use of a nitrophenylalanine or a BOC-amino group, which are optionally converted to carboxyl groups. Synthetic routes to these scaffolds are shown in Schemes 1.3 and 1.4.

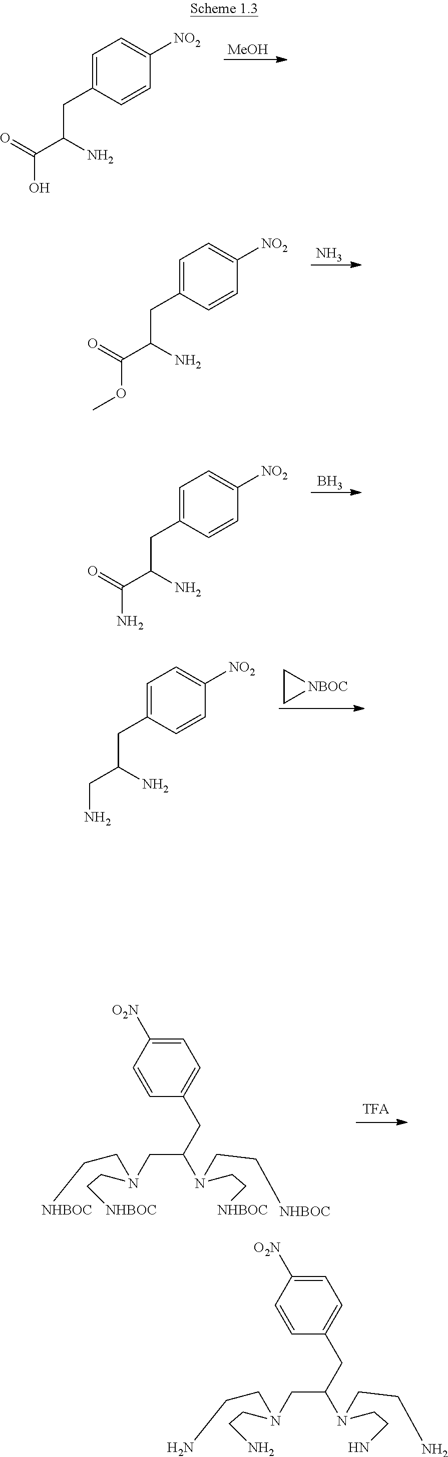

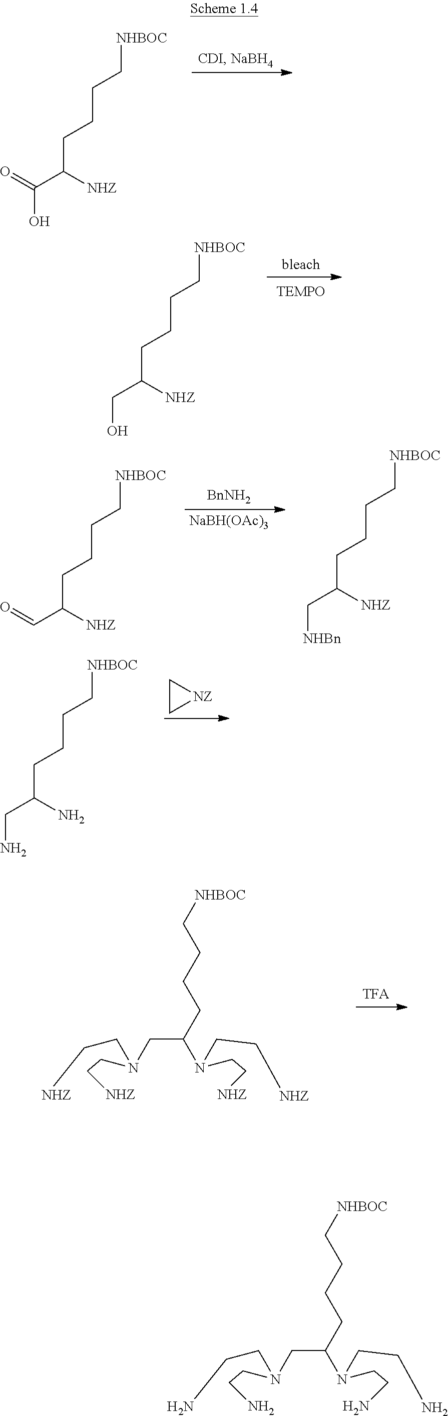

One concern with HOPO chelating moieties is that it might be difficult to couple these to a targeting moiety, such as an antibody, without protection in some form or another. One approach for HOPO chelating moiety protection/deprotection is to use a metal complex in the coupling reaction, then remove the metal from the metal complex-antibody conjugate after coupling to make room for the radionuclide (transmetalation). Another approach is to use ortho-nitrobenzyl in place of the benzyl protective group in the HOPO chelating moiety synthesis, and photodeprotect this after coupling the potential chelating moiety to the antibody.

Additional guidance for deprotecting, activating and attaching one or more chelating moieties to one or more scaffolds can be found, for example in U.S. Pat. Nos. 5,624,901; 6,406,297; 6,515,113 and 6,846,915; US Patent Application Publications 2008/0213780; 2008/0213917 and 2010/0015725; and PCT/US2010/046517.

It is to be understood that the compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers. Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

In each of the foregoing and each of the following embodiments, it is also to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to be a description of such hydrates and/or solvates, including pharmaceutically acceptable solvates.

In each of the foregoing and each of the following embodiments, it is also to be understood that the formulae include and represent each possible isomer, such as stereoisomers and geometric isomers, both individually and in any and all possible mixtures. In each of the foregoing and each of the following embodiments, it is also to be understood that the formulae include and represent any and all crystalline forms, partially crystalline forms, and non crystalline and/or amorphous forms, and co-crystals of the compounds.

Exemplary macrocycles, any of which can be derivatized with a linker (e.g., a functionalized linker or a linker comprising a targeting moiety) are disclosed throughout the application.

The following examples are intended to further illustrate the invention and are intended solely for illustration and are non-limiting.

### EXAMPLES

The compounds and complexes of the invention are synthesized by an appropriate combination of generally well-known synthetic methods. Techniques useful in synthesizing the compounds of the invention are both readily apparent and accessible to those of skill in the relevant art. The discussion below is offered to illustrate certain of the diverse methods available for use in assembling the compounds of the invention, it is not intended to limit the scope of reactions or reaction sequences that are useful in preparing the compounds of the present invention.

### Example 1: Synthesis of a PSMA-Targeting Ligand-Macrocyclic Metal Chelator Conjugate (Scheme 1)

### Lumi4-Conjugate 3

Lumi4-NHS 1 and (S)-2-[3-((S)-5-amino-1-tert-butoxycarbonylpentyl)ureido]pentanedioic acid di-tert-butyl ester 2 were synthesized as previously described (1,2). (S)-2-[3-((S)-5-Amino-1-tert-butoxycarbonylpentyl)ureido]pentanedioic acid di-tert-butyl ester 2 (39 mg, 80 μmol) was dissolved in anhydrous dimethylformamide (DMF, 200 μL) and triethylamine (TEA, 11 μL, 80 μmol). This solution was added to Lumi4-NHS 1 (11.4 mg, 7.88 μmol) and the resulting solution was shaken under a nitrogen atmosphere for 1.5 hours. The solution was transferred to a 10 mL round bottom flask using 1 mL methanol, solvents were removed at reduced pressure, and the residue was dried overnight in vacuo. The residue was dissolved in a solution (1 mL) of 5% triisopropyl silane (TIPS) and 50% trifluoroacetic acid (TFAA) in dichloromethane (DCM) and the resulting solution was stirred for 4 hours. Solvents were removed in vacuo, and the residue was transferred using methanol (500 μL) to a 2 mL O-ring capped microcentrifuge tube. Diethyl ether (1.5 mL) was added to form a precipitate, which was centrifuged at 13,500 rpm for 3 minutes. The supernatant was removed, and the pellet was washed with diethyl ether (1.5 mL). The pellet was dried in vacuo, dissolved in methanol (500 μL), and 0.1% TFAA (500 μL) was added. The crude product was purified by high performance liquid chromatography (HPLC) using a reverse phase column and a mobile phase consisting of 0.1% TFAA and a gradient of 20% to 30% (v/v) acetonitrile. Fractions containing product were combined, solvent removed by freeze drying, and the resulting solid was dissolved in endotoxin-free water (2 mL). A sample was quantified by UV-vis spectroscopy using the extinction coefficient for the Lumi4 tri-macrocycle (26,000 M⁻¹ cm⁻¹) at 347 nm in 50 mM tris(hydroxymethyl) aminomethane (TRIS) buffered saline (TBS), pH 7.4. Quantitation by UV-vis indicated a yield of 4.6 μmol product (58% based on Lumi4-NHS 1). HPLC of the product indicated the presence of two overlapping peaks at retention times of 14.8 minutes (26%) and 15.0 minutes (73%). FTMS+pESI. Calc. for C₇₃H₉₉N₁₆O₂₁ (M+H)⁺, 1535.7165; found, 1535.7168.

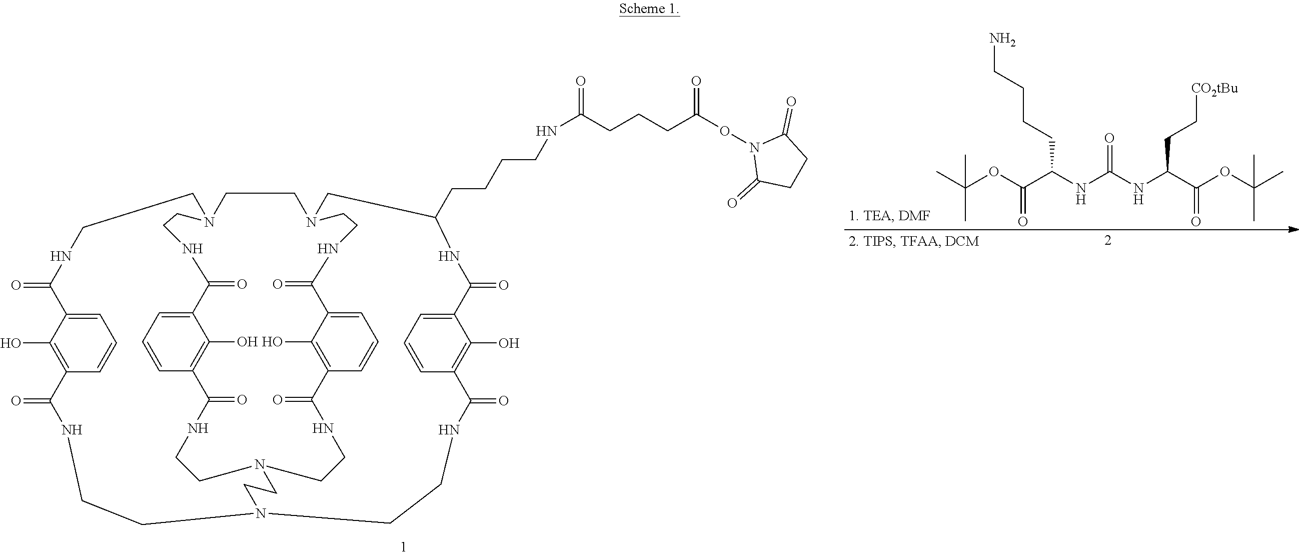 [SMILES: O=C(CCCC(=O)ON1C(=O)CCC1=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2 ; CC(C)(C)OC(=O)CC[C@H](NC(=O)N[C@@H](CCCCN)C(=O)OC(C)(C)C)C(=O)OC(C)(C)C]

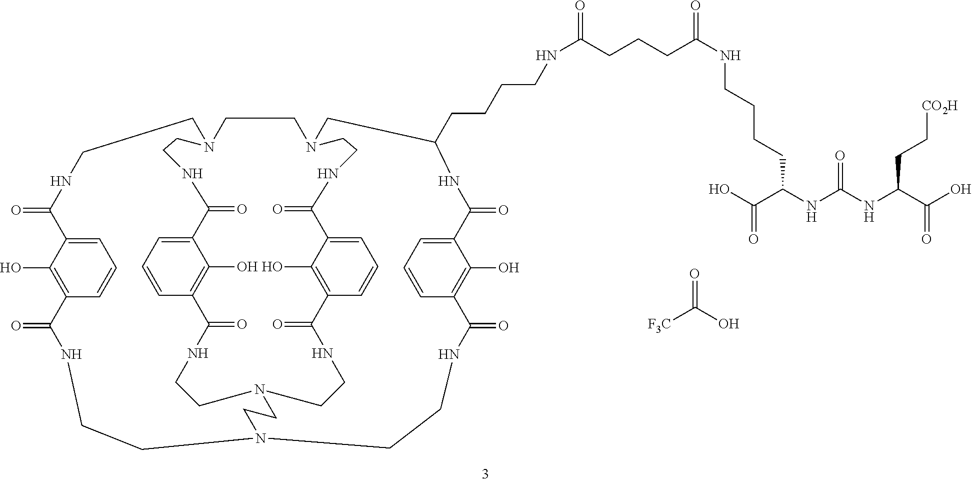 [SMILES: O=C(O)CC[C@H](NC(=O)N[C@@H](CCCCNC(=O)CCCC(=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2)C(=O)O)C(=O)O ; O=C(O)C(F)(F)F]

### Example 2: Synthesis of a LHRH-Targeting Ligand-Macrocyclic Metal Chelator Conjugate (Scheme 2)

### Lumi4-Conjugate 5

Luteinizing hormone releasing hormone analogue ((D-Lys6)-LHR, Aeterna-Zentaris GmbH, Frankfurt, 18.4 mg, 11.5 μmol) was dissolved in anhydrous DMF (200 μL) in a 2 mL O-ring microcentrifuge tube. Triethylamine (TEA, 15.8 μL, 114 μmol) was added. The resulting solution was added to Lumi4-NHS 1 (10.9 mg, 7.55 μmol), mixed, divided between two tubes, and shaken under an inert atmosphere for 3 hours. Trifluoroacetic acid (8 μL per tube) was then added, the solution vortexed briefly, and diethyl ether (1.7 mL per tube) was added to form a precipitate. The tubes were centrifuged, decanted, and the pellets washed with ether (2 mL). After drying, the pellets were dissolved in 0.1% trifluoroacetic acid (0.5 mL per tube) and the resulting solutions were subjected to reverse phase HPLC. The product fractions were collected, combined, and lyophilized. The product was dissolved in endotoxin free water (3 mL). Quantitation by UV-vis indicated a yield of 5.1 μmol (67% based on Lumi4-NHS 1). HPLC of the product indicated single peak at a retention time of 15.9 minutes (98%). FTMS+pESI. Calc. for C₁₂₀H₁₆₃N₃₁O₂₇ (M+2H)²⁺, 1235.1162; found, 1235.1173.

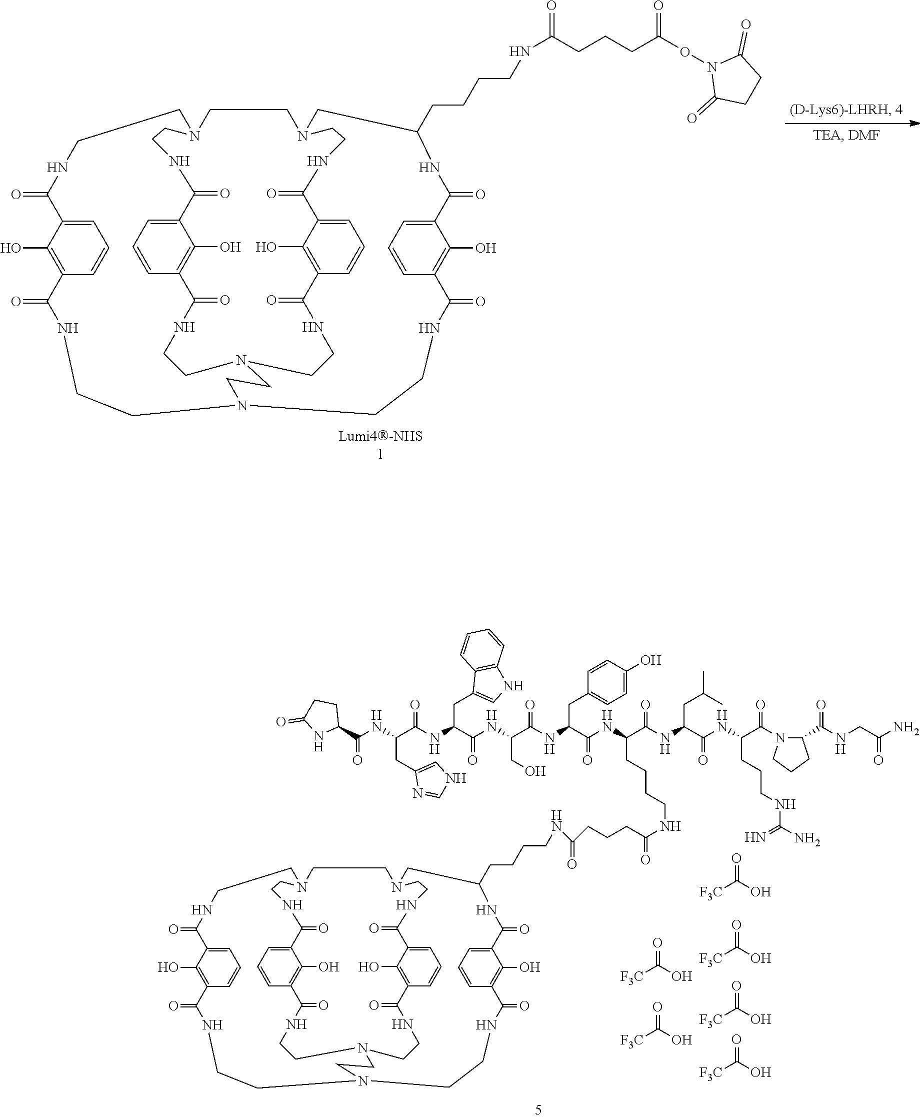

### Example 3: Synthesis of a PSMA-Targeting Ligand-Macrocyclic Metal Chelator Conjugate (Scheme 3)

### tert-Butyl (4-(2-thioxothiazolidine-3-carbonyl)benzyl)carbamate 7

4-(((tert-Butoxycarbonyl)amino)methyl)benzoic acid 6 (1000 mg, 3.98 mmol), 1-[bis(dimethylamino)methylene]-1H-1,2,3-triazolo[4,5-b]pyridinium 3-oxide hexafluorophosphate (HATU, 1.891 g, 4.97 mmol), and 4-dimethylaminopyridine (DMAP, 49 mg, 0.4 mmol) were dried together overnight in vacuo. 2-Mercaptothiazoline (712 mg, 5.97 mmol) was dried separately in vacuo. The mixture of 6, HATU, and DMAP was suspended in dry DCM (20 mL), and diisopropylethylamine (DIPEA, 690 μL, 3.98 mmol) was added. After 30 minutes, 2-mercaptothiazoline and additional DIPEA (1.39 mL, 7.96 mmol) were added, and the resulting solution was stirred for 90 minutes. The volume of the organic layer was reduced under low pressure. The crude product was purified by silica gel chromatography using 1% isopropyl alcohol in dichloromethane to elute the product. Solvents were removed under reduced pressure, and the residue was dried in vacuo to provide 1.29 g (92.0%) compound 7 as a yellow solid.

### di-tert-Butyl (((S)-1-(tert-butoxy)-6-(4-(((tert-butoxycarbonyl)amino)methyl)benzamido)-1-oxohexan-2-yl)carbamoyl)-L-glutamate 8

(S)-2-[3-((S)-5-Amino-1-tert-butoxycarbonylpentyl)ureido]pentanedioic acid di-tert-butyl ester 2 (38 mg, 78 μmol) and tert-butyl (4-(2-thioxothiazolidine-3-carbonyl)benzyl)carbamate 7 (33 mg, 94 μmol) were combined in a flask, dichloromethane (1 mL) and triethylamine (33 μL, 237 μmol) were added, and the solution was stirred at ambient temperature overnight. The solution was concentrated under reduced pressure, and the product was purified by silica gel chromatography using 2% isopropyl alcohol in dichloromethane to elute the product. Solvents were removed under reduced pressure, and the residue was dried in vacuo to provide 50 mg (89%) compound 8.

### (((S)-5-(4-(Aminomethyl)benzamido)-1-carboxypentyl)carbamoyl)-L-glutamic Acid 9

di-tert-Butyl (((S)-1-(tert-butoxy)-6-(4-(((tert-butoxycarbonyl)amino)methyl)benzamido)-1-oxohexan-2-yl)carbamoyl)-L-glutamate 8 (47 mg, 65 μmol) was dissolved in a solution (1.2 mL) of 5% triisopropyl silane (TIPS) and 50% trifluoroacetic acid in dichloromethane and the resulting solution was stirred for 3.5 hours. The solvents were removed under reduced pressure, and the product was dried in vacuo overnight. The residue was dissolved in dimethylformamide (300 μL) and used in the next step without further purification.

### Lumi4-Conjugate 10

Lumi4-NHS 1 (8.04 mg, 5.61 μmol) and (((S)-5-(4-(aminomethyl)benzamido)-1-carboxypentyl)carbamoyl)-L-glutamic acid 9 (200 μL of the solution prepared above, ca. 43 μmol) were combined in a microcentrifuge tube. Triethylamine (16.2 μL, 116 μmol) was added, and the resulting suspension was shaken overnight at 1200 rpm. The suspension was dissolved by the addition of methanol (500 μL) and trifluoroacetic acid (10 μL). The crude product was purified by high performance liquid chromatography (HPLC) using a reverse phase column and a mobile phase consisting of 0.1% TFAA and a gradient of 22% to 30% (v/v) acetonitrile. Fractions containing product were combined, solvent removed by freeze drying, and the resulting solid was dissolved in endotoxin-free water (2 mL). A sample was quantified by UV-vis spectroscopy using the extinction coefficient for the Lumi4 tri-macrocycle (26,000 M⁻¹ cm⁻¹) at 347 nm in 50 mM tris(hydroxymethyl) aminomethane (TRIS) buffered saline (TBS), pH 7.4. Quantitation by UV-vis indicated a yield of 2.3 μmol (40% based on Lumi4-NHS 1). HPLC of the product indicated the presence of one peak at retention time of 15.6 minutes (100%). FTMS+pESI. Calc. for C₈₁H₁₀₇N₁₇O₂₂ (M+2H)²⁺, 834.8883; found, 834.8884.

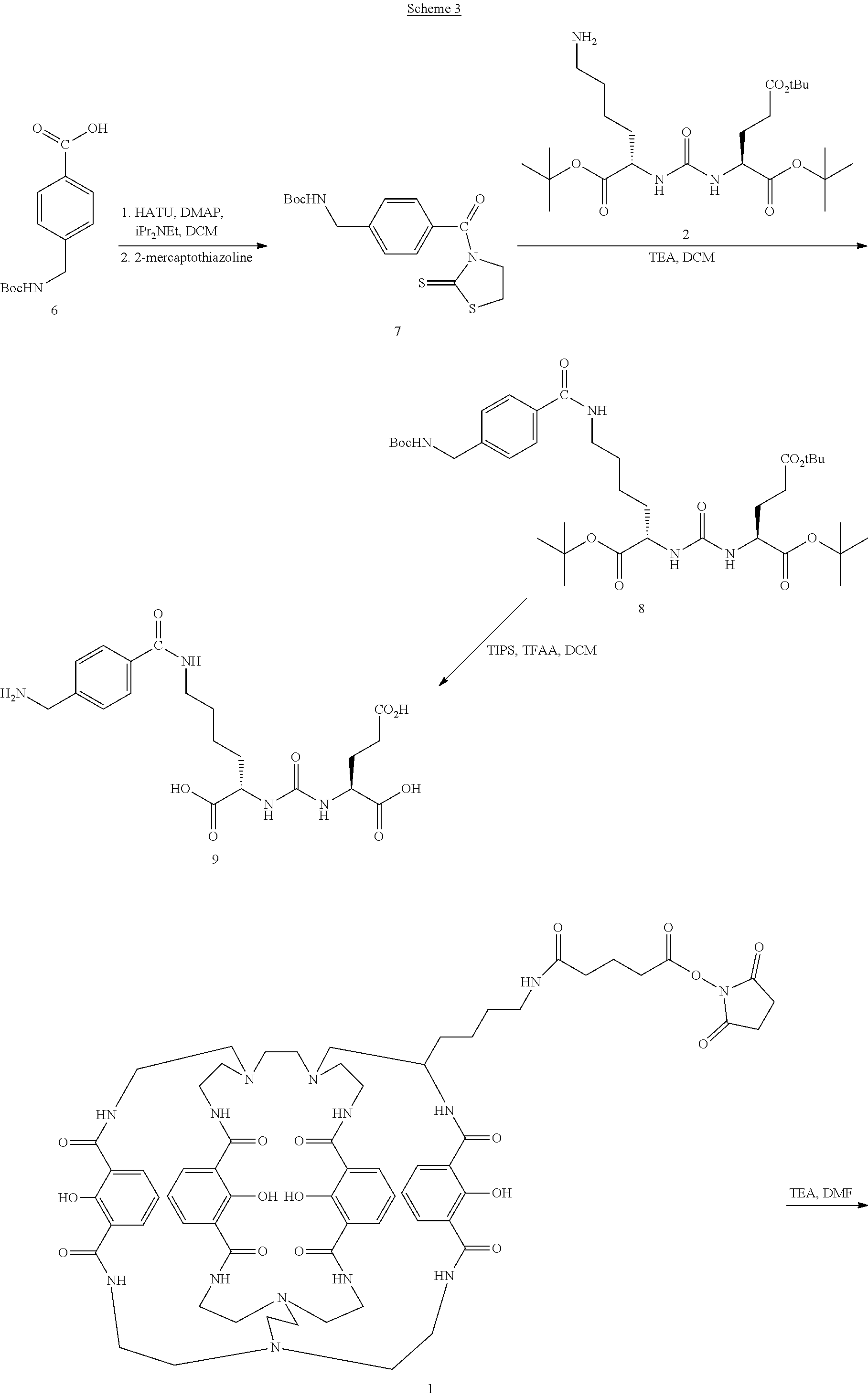 [SMILES: CC(C)(C)OC(=O)NCc1ccc(C(=O)O)cc1 ; CC(C)(C)OC(=O)NCc1ccc(C(=O)N2CCSC2=S)cc1 ; CC(C)(C)OC(=O)CC[C@H](NC(=O)N[C@@H](CCCCN)C(=O)OC(C)(C)C)C(=O)OC(C)(C)C ; CC(C)(C)OC(=O)CC[C@H](NC(=O)N[C@@H](CCCCNC(=O)c1ccc(CNC(=O)OC(C)(C)C)cc1)C(=O)OC(C)(C)C)C(=O)OC(C)(C)C ; NCc1ccc(C(=O)NCCCC[C@H](NC(=O)N[C@@H](CCC(=O)O)C(=O)O)C(=O)O)cc1 ; O=C(CCCC(=O)ON1C(=O)CCC1=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2]

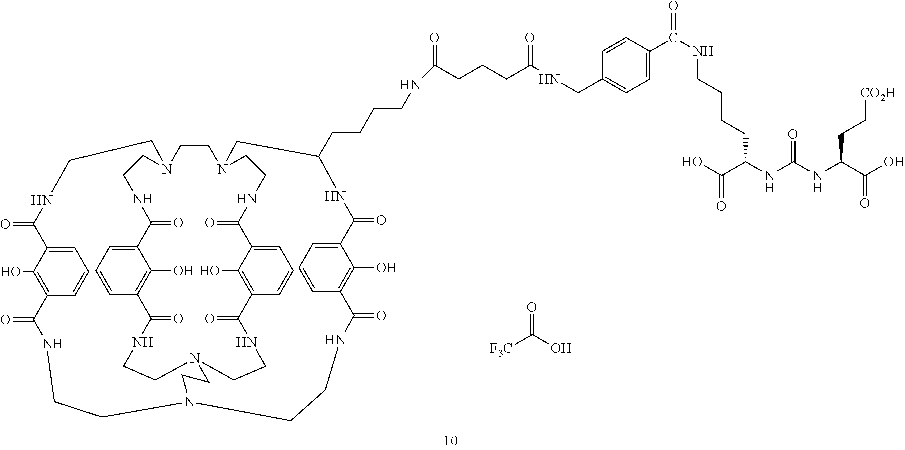 [SMILES: O=C(O)CC[C@H](NC(=O)N[C@@H](CCCCNC(=O)c1ccc(CNC(=O)CCCC(=O)NCCCCC2CN3CCNC(=O)c4cccc(c4O)C(=O)NCCN4CCNC(=O)c5cccc(c5O)C(=O)NCCN(CCNC(=O)c5cccc(c5O)C(=O)NCCN(CCNC(=O)c5cccc(c5O)C(=O)N2)CC4)CC3)cc1)C(=O)O)C(=O)O ; O=C(O)C(F)(F)F]

### Example 4: Synthesis of a PSMA-Targeting Ligand-Macrocyclic Metal Chelator Conjugate (Scheme 4)

### (8R,9S,13S,14S,17S)-13-Methyl-3-(2-(2-(2-(2-(tosyloxy)ethoxy)ethoxy)ethoxy)ethoxy)-7,8,9,11,12,13,14,15,16,17-decahydro-6H-cyclopenta[a]phenanthren-17-yl Acetate 13

β-Estradiol 17-acetate 11, tetraethylene glycol di(p-toluenesulfonate) 12, and potassium carbonate are combined in a flask. Acetonitrile is added, and the mixture is heated at reflux. After cooling, solvent is removed under reduced pressure. The residue is dissolved in dichloromethane, washed with water, and the crude product is purified using silica gel chromatography.

### tert-Butyl (S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanoate 15

(8R,9S,13S,14S,17S)-13-methyl-3-(2-(2-(2-(2-(tosyloxy)ethoxy)ethoxy)ethoxy)ethoxy)-7,8,9,11,12,13,14,15,16,17-decahydro-6H-cyclopenta[a]phenanthren-17-yl acetate 13, Z-L-tyrosine tert-butyl ester 14, and potassium carbonate are combined in a flask. Acetonitrile is added, and the mixture is heated at reflux. After cooling, solvent is removed under reduced pressure. The residue is dissolved in dichloromethane, washed with water, and the crude product is purified using silica gel chromatography.

### 2,5-Dioxopyrrolidin-1-yl (S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanoate 16

tert-Butyl (S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanoate 15 is dissolved in a solution of 5% triisopropyl silane (TIPS) and 50% trifluoroacetic acid (TFAA) in dichloromethane (DCM) and the resulting solution is stirred for 4 hours. The solvents are removed under reduced pressure, and the residue is dissolved in anhydrous tetrahydrofuran. N-hydroxysuccinimide is added, and a 1M solution of N,N-dicyclohexylcarbodiimide in dichloromethane is added thereafter. After stirring, the suspension is filtered, and solvent is reduced in volume under reduced pressure. The residue is dissolved in DCM and diethyl ether is added to form a precipitate. The precipitate is filtered, dissolved in dichloromethane, and diethyl ether is again added to form a precipitate. The product is collected and dried in vacuo.

### di-tert-Butyl (((S)-6-((S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanamido)-1-(tert-butoxy)-1-oxohexan-2-yl)carbamoyl)-L-glutamate 17

(S)-2-[3-((S)-5-Amino-1-tert-butoxycarbonylpentyl)ureido]pentanedioic acid di-tert-butyl ester 2 is dissolved in dichloromethane and triethylamine. 2,5-Dioxopyrrolidin-1-yl (S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanoate 16 is added, and the solution is stirred at ambient temperature. The solution is concentrated under reduced pressure, and the product is purified using silica gel chromatography.

### di-tert-Butyl (((S)-6-((S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-aminopropanamido)-1-(tert-butoxy)-1-oxohexan-2-yl)carbamoyl)-L-glutamate 18

di-tert-Butyl (((S)-6-((S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-(((benzyloxy)carbonyl)amino)propanamido)-1-(tert-butoxy)-1-oxohexan-2-yl)carbamoyl)-L-glutamate 17 is dissolved in ethanol. Palladium on carbon (10%) catalyst is added, and the atmosphere is exchanged five times for hydrogen gas using a balloon. After stirring overnight the catalyst is filtered using Celite© filter aid, the filtrate is concentrated under reduced pressure, and the product is dried in vacuo.

### Lumi4-Conjugate 19

Lumi4-NHS 1 and di-tert-butyl (((S)-6-((S)-3-(4-(2-(2-(2-(2-(((8S,9R,13R,14R,17R)-17-acetoxy-13-methyl-7,8,9,11,12,13,14,15,16,17-decahydro-6H-cyclopenta[a]phenanthren-3-yl)oxy)ethoxy)ethoxy)ethoxy)ethoxy)phenyl)-2-aminopropanamido)-1-(tert-butoxy)-1-oxohexan-2-yl)carbamoyl)-L-glutamate 18 are reacted as described above in the preparation of conjugate 3, except that the initially formed residue is dissolved in methanol, sodium hydroxide solution is added, and the solution is stirred. Solvents are removed under reduced pressure, and the residue is dissolved in a solution of 5% triisopropyl silane (TIPS) and 50% trifluoroacetic acid (TFAA) in dichloromethane (DCM) as described above. Solvents are removed and the residue is dried overnight in vacuo. The residue is transferred using methanol to a 2 mL O-ring capped microcentrifuge tube. Diethyl ether is added to form a precipitate, which is centrifuged at 13,500 rpm for 3 minutes. The supernatant is removed, and the pellet is washed with diethyl ether. The pellet is dried in vacuo, dissolved in methanol, and 0.1% TFAA is added. The crude product is purified by high performance liquid chromatography (HPLC) using a reverse phase column and a mobile phase consisting of 0.1% TFAA and a gradient of acetonitrile. Fractions containing product are combined, solvent removed by freeze drying, and the resulting solid is dissolved in endotoxin-free water to provide a solution of Lumi4-conjugate 19.

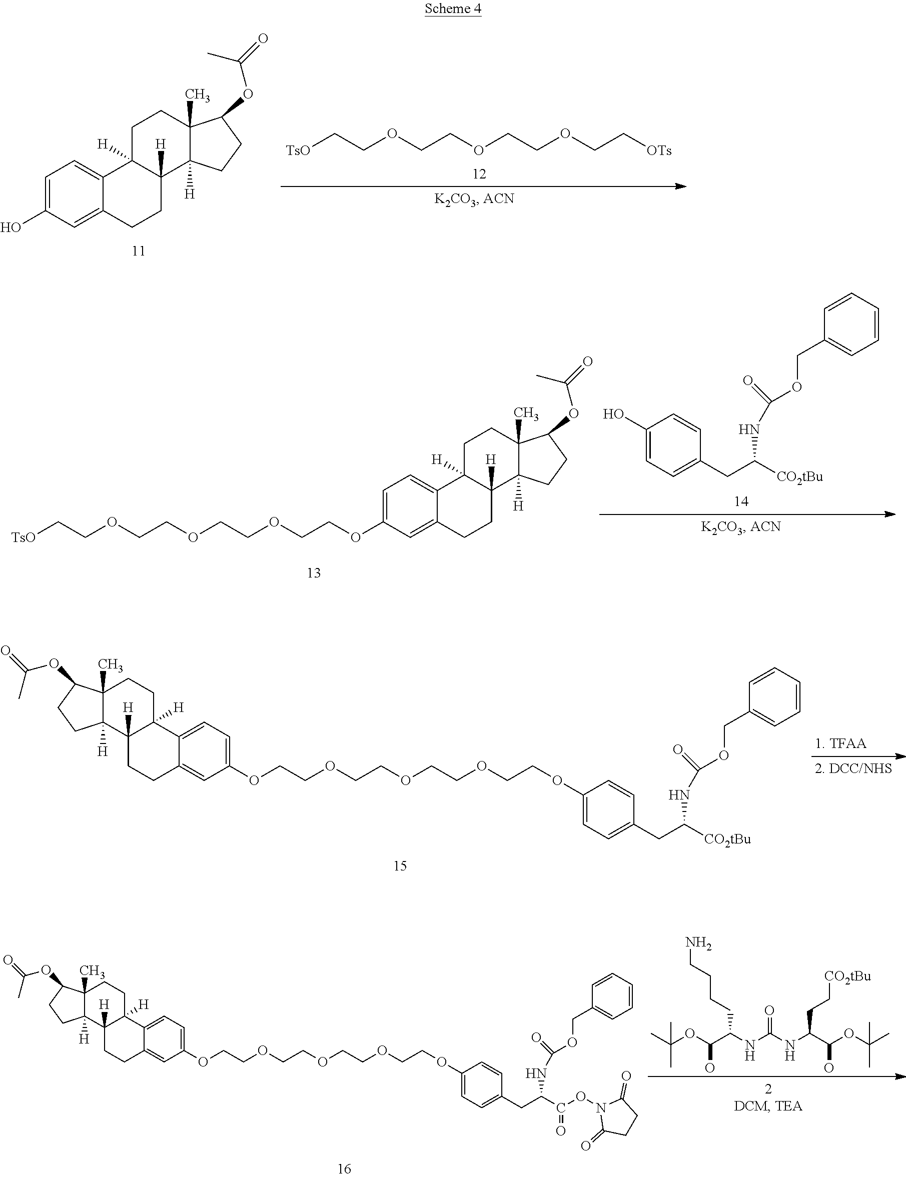

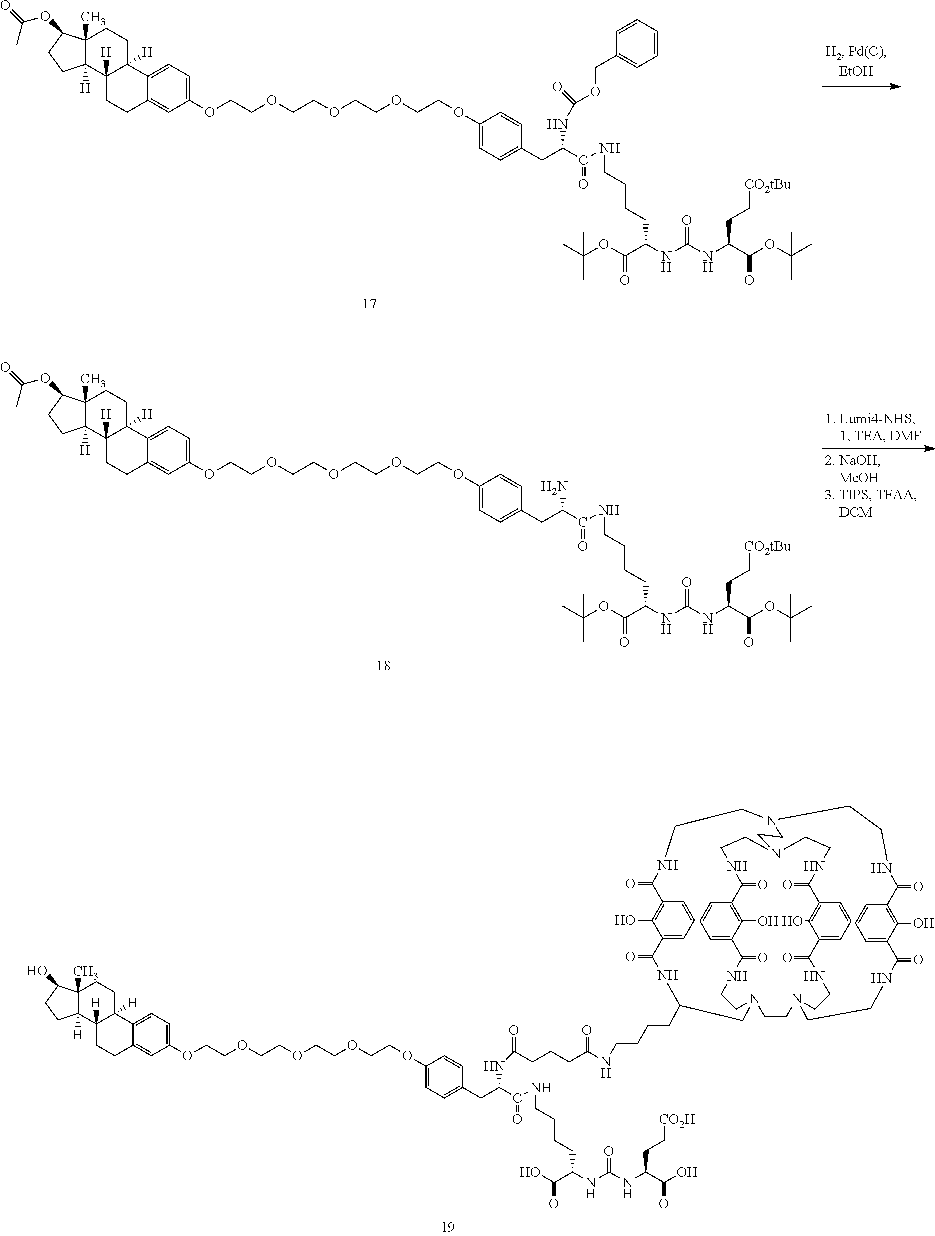

### Example 5: Synthesis of a PSMA-Targeting Ligand-Macrocyclic Metal Chelator Conjugate (Scheme 5)

### Lumi804-NCS 21

Lumi804-NH2 20 was synthesized as previously described (3). Lumi804-NH2 20 (25 mg, 25.8 μmol) was dissolved in DMF (0.3 mL), and added to phenylenediisothiocyanate (49.6 mg, 258 μmol) that was dissolved in DMF (0.5 mL) and TEA (26.1 mg, 258 μmol). The solution was mixed at 1200 rpm for one hour, whereupon the reaction mixture was divided into 200 uL/tube and diethyl ether (1.8 mL/tube) was added to form a precipitate. The mixture was centrifuged for 3 minutes at 12000 rpm, the supernatant removed, and the precipitate was washed with diethyl ether (ca. 1.7 mL/tube). The mixture was centrifuged, the supernatant was removed, and the pellet was allowed to dry. The pellet was dissolved in DMF (150 L/tube), and diethyl ether (1.8 mL/tube) was added to form a precipitate. The mixture was centrifuged for 3 minutes at 12000 rpm, the supernatant removed, and the precipitate was washed with diethyl ether (ca. 1.7 mL). The mixture was centrifuged, the supernatant was removed, and the pellet was allowed to dry. This precipitation procedure was repeated two additional times to assure complete removal of phenylenediisothiocyanate reagent. The product was dried overnight in vacuo to provide ca. 30 mg of yellow powder. FTMS-pESI. Calc. for C50H54N13O14S2 (M−H)−, 1124.3360; found, 1124.3338.

### Lumi804-Conjugate 23

(S)-2-[3-((S)-5-Amino-1-tert-butoxycarbonylpentyl)ureido]pentanedioic acid di-tert-butyl ester 2 (6.5 mg, 13.3 μmol) was dissolved in a solution (1 mL) of 5% triisopropyl silane (TIPS) and 50% trifluoroacetic acid in dichloromethane and the resulting solution was stirred for 3.5 hours. The solvents were removed under reduced pressure, and the product was dried in vacuo overnight. The intermediate 22 was used without further purification. Compound 22 was dissolved in dimethylformamide (100 μL) and added to a solution of compound 21 (7.5 mg, 6.7 μmol) in DMF (300 μL). Triethylamine (50 μL, 359 μmol) was added, and the resulting suspension was shaken overnight at 1200 rpm. Water (2 mL) was added to the reaction mixture to form a solution. The crude product was purified by HPLC using a reverse phase column and a mobile phase consisting of 0.1% TFAA and a gradient of 10% to 40% (v/v) acetonitrile. The column temperature was 60° C. Fractions containing product were combined, solvent removed by freeze drying, and the resulting solid was dissolved in TBS buffer, pH 7.4 (1 mL). A sample was quantified by UV-vis spectroscopy using the extinction coefficient for the Lumi804 macrocycle (14,300 M⁻¹ cm⁻¹) at 383 nm in TBS buffer, pH 7.4. Quantitation by UV-vis indicated a yield of 2.15 μmol (32% based on Lumi804-NCS 21). FTMS-pESI. Calc. for C₆₂H₇₅N₁₆O₂₁S₂ (M−H)⁻, 1443.4740; found, 1443.4713.

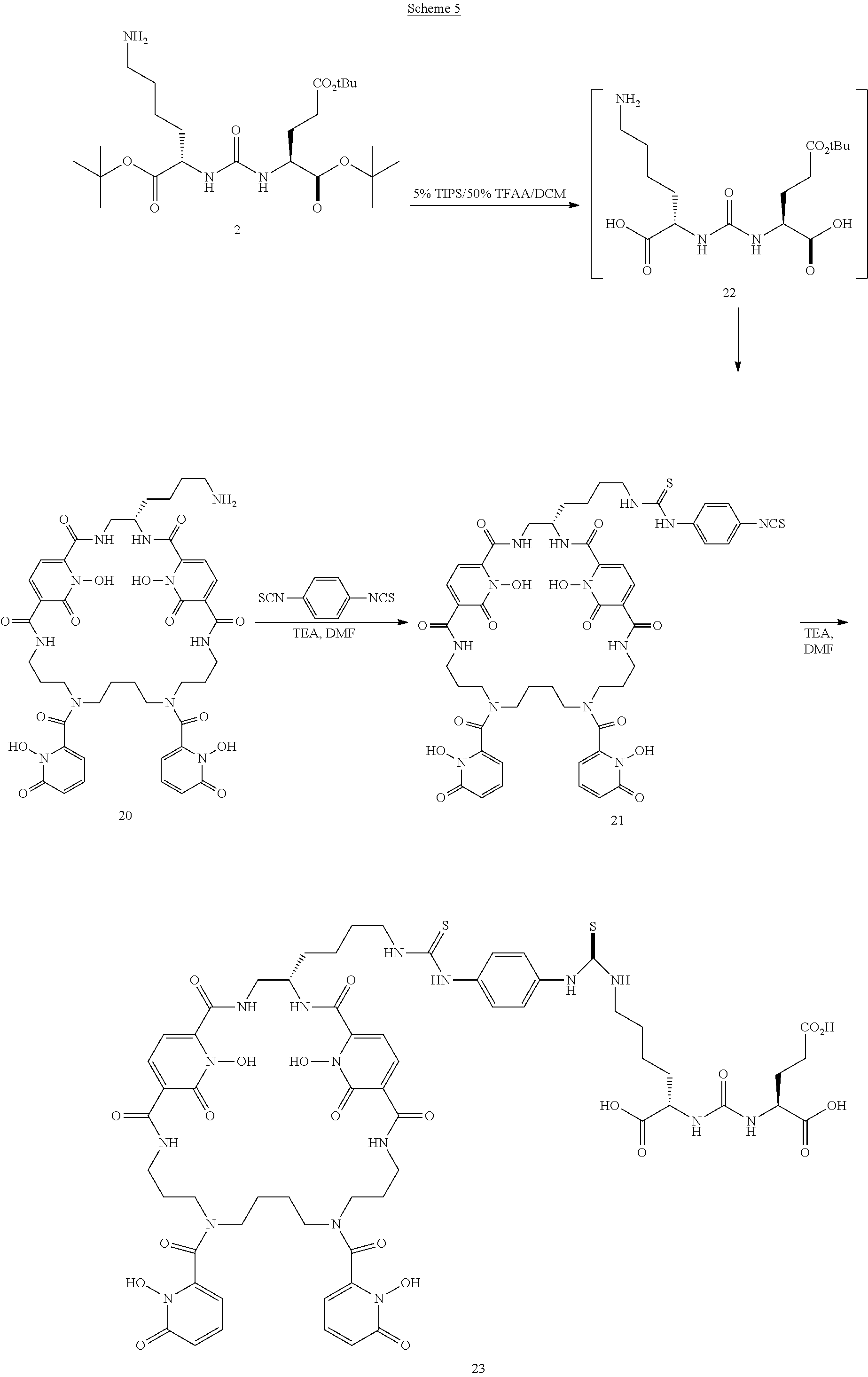

### Example 6: Synthesis of a PSMA-Targeting Ligand-Macrocyclic Metal Chelator Conjugate (Scheme 6)

### Lumi4-Conjugate 25

### 2-(((S)-((4R)-4-(4-(6-Aminohexanamido)-4-carboxybutanamido)-4-carboxy butoxy) (hydroxy)phosphoryl)amino)pentanedioic Acid 24 is Prepared as Described⁴

2-(((S)-((4R)-4-(4-(6-Aminohexanamido)-4-carboxybutanamido)-4-carboxy butoxy) (hydroxy)phosphoryl)amino)pentanedioic acid 24 is dissolved in anhydrous DMF and TEA. This solution is added to Lumi4-NHS 1 and the resulting solution is shaken under a nitrogen atmosphere. Solvents are removed and the residue is dried overnight in vacuo. The residue is transferred using methanol to a 2 mL O-ring capped microcentrifuge tube. Diethyl ether is added to form a precipitate, which is centrifuged at 13,500 rpm for 3 minutes. The supernatant is removed, and the pellet is washed with diethyl ether. The pellet is dried in vacuo, dissolved in methanol, and 0.1% TFAA is added. The crude product is purified by high performance liquid chromatography (HPLC) using a reverse phase column and a mobile phase consisting of 0.1% TFAA and a gradient of acetonitrile. Fractions containing product are combined, solvent removed by freeze drying, and the resulting solid is dissolved in endotoxin-free water to provide a solution of Lumi4-conjugate 25.

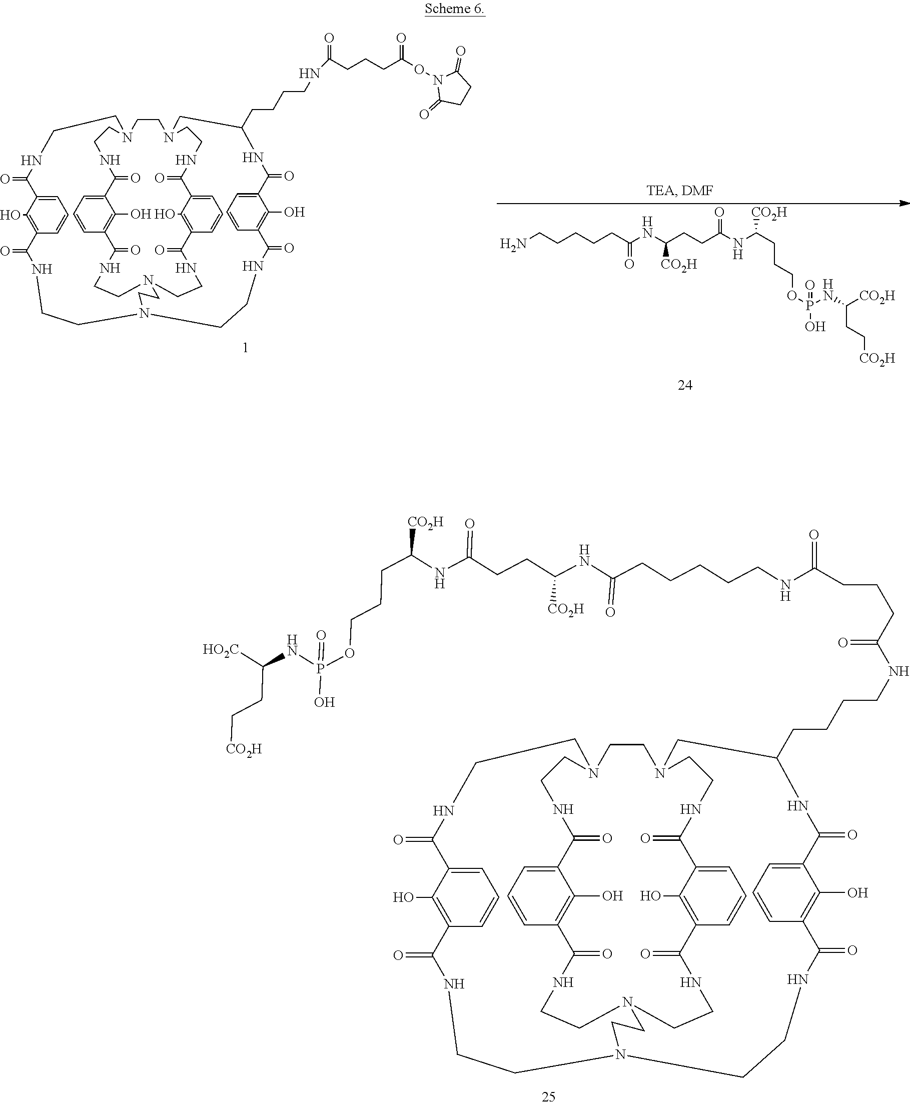 [SMILES: O=C(CCCC(=O)ON1C(=O)CCC1=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2 ; NCCCCCC(=O)N[C@@H](CCC(=O)N[C@@H](CCCOP(=O)(O)N[C@@H](CCC(=O)O)C(=O)O)C(=O)O)C(=O)O ; O=C(O)CC[C@H](NP(=O)(O)OCCC[C@H](NC(=O)CC[C@H](NC(=O)CCCCCNC(=O)CCCC(=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2)C(=O)O)C(=O)O)C(=O)O]

### Example 7: Synthesis of a PSMA-Targeting Ligand-Macrocyclic Metal Chelator Conjugate (Scheme 7)

Lumi4-azide 26. Lumi4-NHIS 1 is dissolved in DMF and TEA. A solution of 3-azido-1-propanamine is added, and the resulting solution is shaken under a nitrogen atmosphere. Solvents are removed and the residue is dried overnight in vacuo. The residue is transferred using methanol to a 2 mL O-ring capped microcentrifuge tube. Diethyl ether is added to form a precipitate, which is centrifuged at 13,500 rpm for 3 minutes. The supernatant is removed, and the pellet is washed with diethyl ether. The residue is dissolved in neutral ammonium acetate buffer and used in the next step without further purification.

### Lumi4-Conjugate 28

CTT1402 27 is prepared as described.⁵ CTT1402 27 is dissolved in neutral ammonium acetate buffer. This solution is added to Lumi4-azide 26 and the resulting solution is warmed to 37° C. The crude product is purified by high performance liquid chromatography (HPLC) using a reverse phase column and a mobile phase consisting of 0.1% TFAA and a gradient of acetonitrile. Fractions containing product are combined, solvent removed by freeze drying, and the resulting solid is dissolved in endotoxin-free water to provide a solution of Lumi4-conjugate 28.

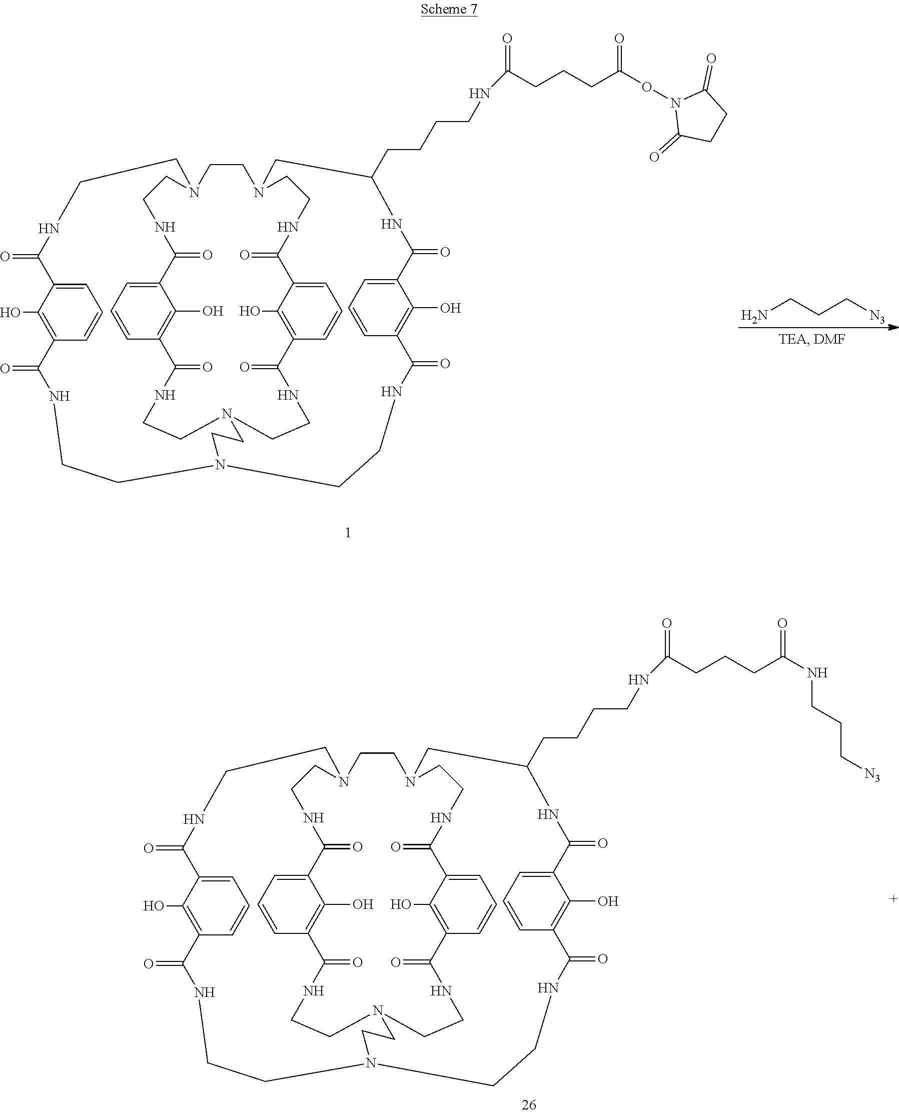 [SMILES: O=C(CCCC(=O)ON1C(=O)CCC1=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2 ; [N-]=[N+]=NCCCN ; [N-]=[N+]=NCCCNC(=O)CCCC(=O)NCCCCC1CN2CCNC(=O)c3cccc(c3O)C(=O)NCCN3CCNC(=O)c4cccc(c4O)C(=O)NCCN(CCNC(=O)c4cccc(c4O)C(=O)NCCN(CCNC(=O)c4cccc(c4O)C(=O)N1)CC3)CC2]

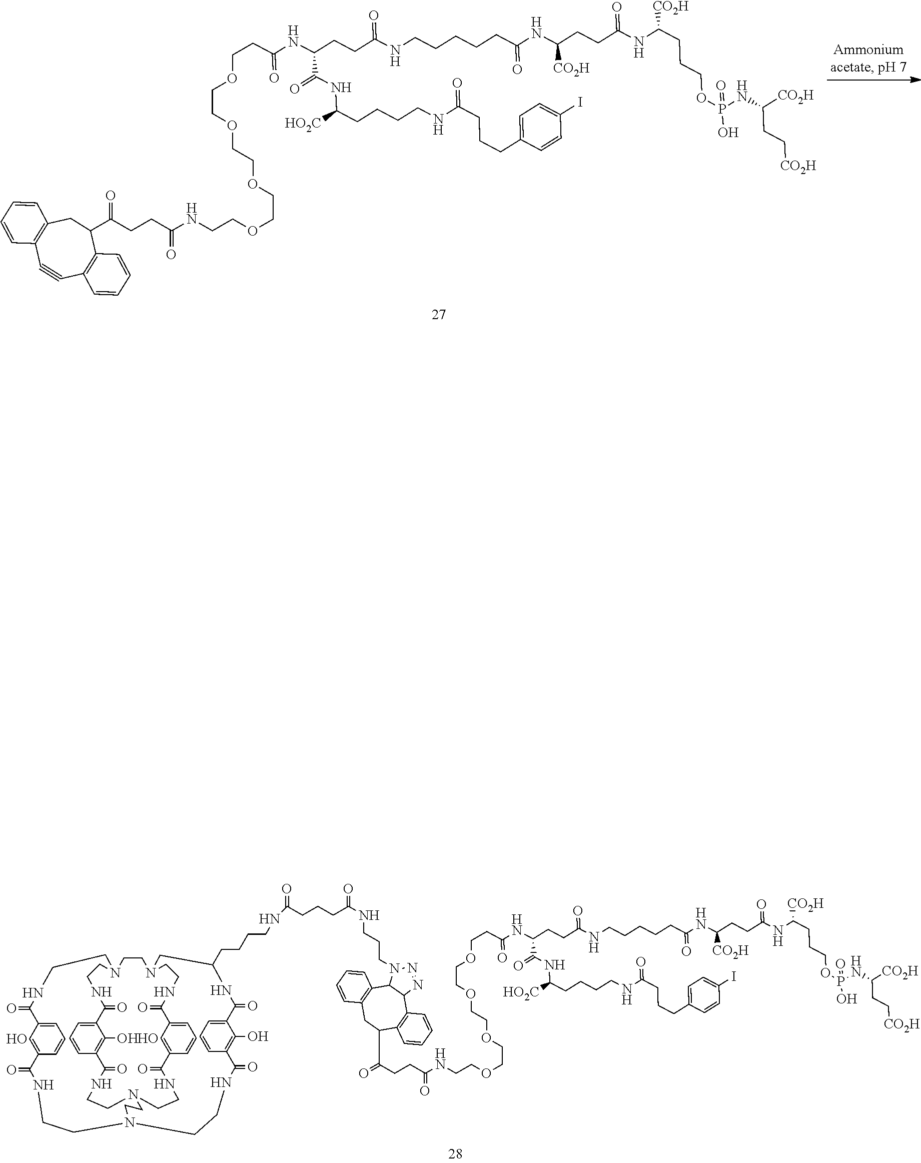

### Example 8: Synthesis of a PSMA-Targeting Ligand-Macrocyclic Metal Chelator Conjugate (Scheme 8)

### 2,5-Dioxopyrrolidin-1-yl (1r,4r)-4-(((((9H-fluoren-9-yl)methoxy)carbonyl)amino)methyl)cyclohexane-1-carboxylate 30

(1r,4r)-4-(((((9H-fluoren-9-yl)methoxy)carbonyl)amino)methyl)cyclohexane-1-carboxylic acid 29 and N-hydroxysuccinimide (NHS) are dried together overnight in vacuo. The compound mixture is dissolved in dry tetrahydrofuran (THF), and dicyclohexylcarbodiimide (DCC) is added. After several hours, the dicyclohexylurea precipitate is filtered, washed with THF, and the volume of the combined filtrates is reduced under low pressure. The crude product is purified by recrystallization from dry DCM and ethyl ether.

### di-tert-Butyl (((S)-6-((1r,4S)-4-(((((9H-fluoren-9-yl)methoxy)carbonyl)amino)methyl)cyclohexane-1-carboxamido)-1-(tert-butoxy)-1-oxohexan-2-yl)carbamoyl)-L-glutamate 31

(S)-2-[3-((S)-5-Amino-1-tert-butoxycarbonylpentyl)ureido]pentanedioic acid di-tert-butyl ester 2 and 2,5-dioxopyrrolidin-1-yl (1r,4r)-4-(((((9H-fluoren-9-yl)methoxy)carbonyl)amino)methyl)cyclohexane-1-carboxylate 30 are combined in a flask, dichloromethane and diisopropylethylamine are added, and the solution is stirred at ambient temperature. The solution is concentrated under reduced pressure, and the product is purified by silica gel chromatography using methyl alcohol in dichloromethane to elute the product. Solvents are removed under reduced pressure, and the residue is dried in vacuo.

### di-tert-Butyl (((S)-6-((1r,4S)-4-(aminomethyl)cyclohexane-1-carboxamido)-1-(tert-butoxy)-1-oxohexan-2-yl)carbamoyl)-L-glutamate 32

di-I-Butyl (((S)-6-((1r,4S)-4-(((((9H-fluoren-9-yl)methoxy)carbonyl)amino)methyl)cyclohexane-1-carboxamido)-1-(tert-butoxy)-1-oxohexan-2-yl)carbamoyl)-L-glutamate 31 is treated with 20% piperidine in DCM. Solvents are removed under reduced pressure, and the residue is dried in vacuo. The residue is used in the next step without further purification.

### (((S)-5-((1r,4S)-4-(Aminomethyl)cyclohexane-1-carboxamido)-1-carboxypentyl)carbamoyl)-L-glutamic Acid 33

di-tert-butyl (((S)-6-((1r,4S)-4-(aminomethyl)cyclohexane-1-carboxamido)-1-(tert-butoxy)-1-oxohexan-2-yl)carbamoyl)-L-glutamate 32 is dissolved in a solution of 5% triisopropyl silane (TIPS) and 50% trifluoroacetic acid in dichloromethane and the resulting solution is stirred for several hours. The solvents are removed under reduced pressure, and the product is dried in vacuo. The residue is dissolved in dimethylformamide and used in the next step without further purification.

### Lumi4-Conjugate 34

Lumi4-NHS 1 and (((S)-5-((1r,4S)-4-(aminomethyl)cyclohexane-1-carboxamido)-1-carboxypentyl)carbamoyl)-L-glutamic acid 33 are combined in a microcentrifuge tube. Triethylamine is added, and the resulting suspension is shaken at 1200 rpm. The suspension is dissolved by the addition of methanol and trifluoroacetic acid. The crude product is purified by high performance liquid chromatography (HPLC) using a reverse phase column and a mobile phase consisting of 0.1% TFAA and acetonitrile. Fractions containing product are combined, solvent removed by freeze drying, and the resulting solid is dissolved in endotoxin-free water.

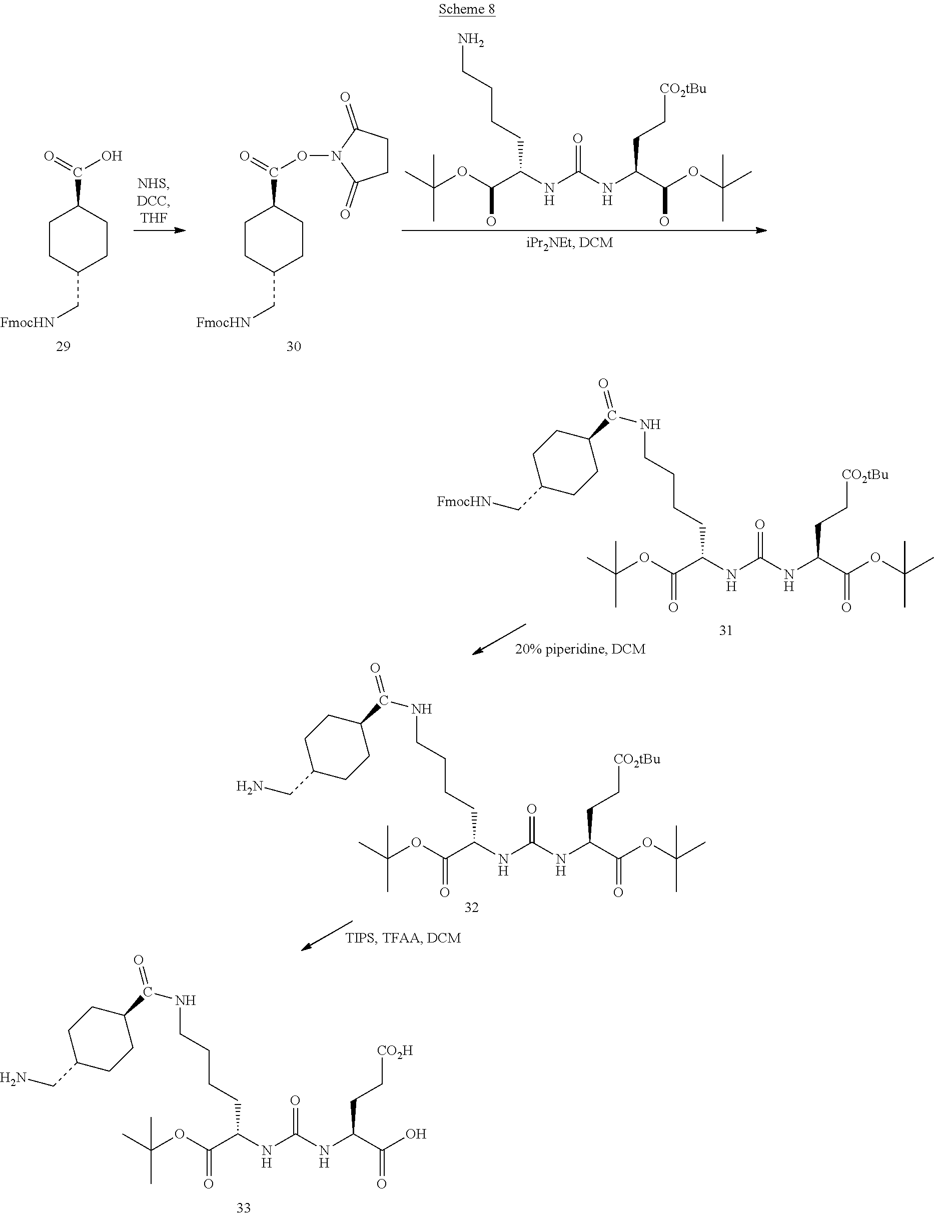

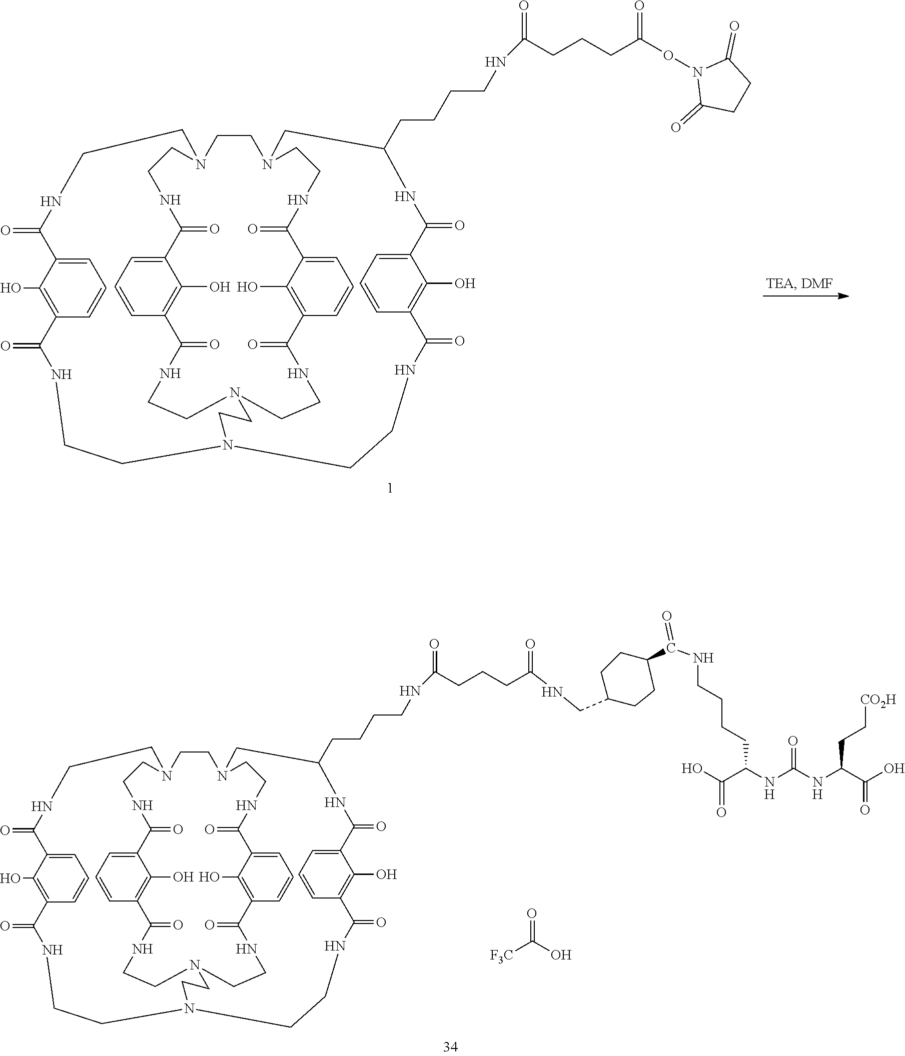

### Example 9: Synthesis of a PSMA-Targeting Ligand-Macrocyclic Metal Chelator Conjugate (Scheme 9)

### 2,5-Dioxopyrrolidin-1-yl (S)-2-((((9H-fluoren-9-yl)methoxy)carbonyl)amino)-3-(naphthalen-1-yl)propanoate 36

(S)-2-((((9H-fluoren-9-yl)methoxy)carbonyl)amino)-3-(naphthalen-1-yl)propanoic acid 35 and N-hydroxysuccinimide (NHS) are dried together overnight in vacuo. The compound mixture is dissolved in dry tetrahydrofuran (THF), and dicyclohexylcarbodiimide (DCC) is added. After several hours, the dicyclohexylurea precipitate is filtered, washed with THF, and the volume of the combined filtrates is reduced under low pressure. The crude product is purified by recrystallization from dry DCM and ethyl ether.

### di-tert-Butyl (((S)-6-((1S,4S)-4-(((S)-2-((((9H-fluoren-9-yl)methoxy)carbonyl)amino)-3-(naphthalen-1-yl)propanamido)methyl)cyclohexane-1-carboxamido)-1-(tert-butoxy)-1-oxohexan-2-yl)carbamoyl)-L-glutamate 37

di-tert-butyl (((S)-6-((1r,4S)-4-(aminomethyl)cyclohexane-1-carboxamido)-1-(tert-butoxy)-1-oxohexan-2-yl)carbamoyl)-L-glutamate 32 and 2,5-dioxopyrrolidin-1-yl (S)-2-((((9H-fluoren-9-yl)methoxy)carbonyl)amino)-3-(naphthalen-1-yl)propanoate 36 are combined in a flask, dichloromethane and diisopropylethylamine are added, and the solution is stirred at ambient temperature. The solution is concentrated under reduced pressure, and the product is purified by silica gel chromatography using methyl alcohol in dichloromethane to elute the product. Solvents are removed under reduced pressure, and the residue is dried in vacuo.

### di-tert-Butyl (((S)-6-((1S,4S)-4-(((S)-2-amino-3-(naphthalen-1-yl)propanamido)methyl)cyclohexane-1-carboxamido)-1-(tert-butoxy)-1-oxohexan-2-yl)carbamoyl)-L-glutamate 38

di-tert-Butyl (((S)-6-((1S,4S)-4-(((S)-2-((((9H-fluoren-9-yl)methoxy)carbonyl)amino)-3-(naphthalen-1-yl)propanamido)methyl)cyclohexane-1-carboxamido)-1-(tert-butoxy)-1-oxohexan-2-yl)carbamoyl)-L-glutamate 37 is treated with 20% piperidine in DCM. Solvents are removed under reduced pressure, and the residue is dried in vacuo. The residue is used in the next step without further purification.

### (((S)-5-((1S,4S)-4-(((S)-2-Amino-3-(naphthalen-1-yl)propanamido)methyl)cyclohexane-1-carboxamido)-1-carboxypentyl)carbamoyl)-L-glutamic Acid 39

di-tert-Butyl (((S)-6-((1S,4S)-4-(((S)-2-amino-3-(naphthalen-1-yl)propanamido)methyl)cyclohexane-1-carboxamido)-1-(tert-butoxy)-1-oxohexan-2-yl)carbamoyl)-L-glutamate 38 is dissolved in a solution of 5% triisopropyl silane (TIPS) and 50% trifluoroacetic acid in dichloromethane and the resulting solution is stirred for several hours. The solvents are removed under reduced pressure, and the product is dried in vacuo. The residue is dissolved in dimethylformamide and used in the next step without further purification.

### Lumi4-Conjugate 40

Lumi4-NHS 1 and (((S)-5-((1S,4S)-4-(((S)-2-amino-3-(naphthalen-1-yl)propanamido)methyl)cyclohexane-1-carboxamido)-1-carboxypentyl)carbamoyl)-L-glutamic acid 39 are combined in a microcentrifuge tube. Triethylamine is added, and the resulting suspension is shaken at 1200 rpm. The suspension is dissolved by the addition of methanol and trifluoroacetic acid. The crude product is purified by high performance liquid chromatography (HPLC) using a reverse phase column and a mobile phase consisting of 0.1% TFAA and acetonitrile. Fractions containing product are combined, solvent removed by freeze drying, and the resulting solid is dissolved in endotoxin-free water.

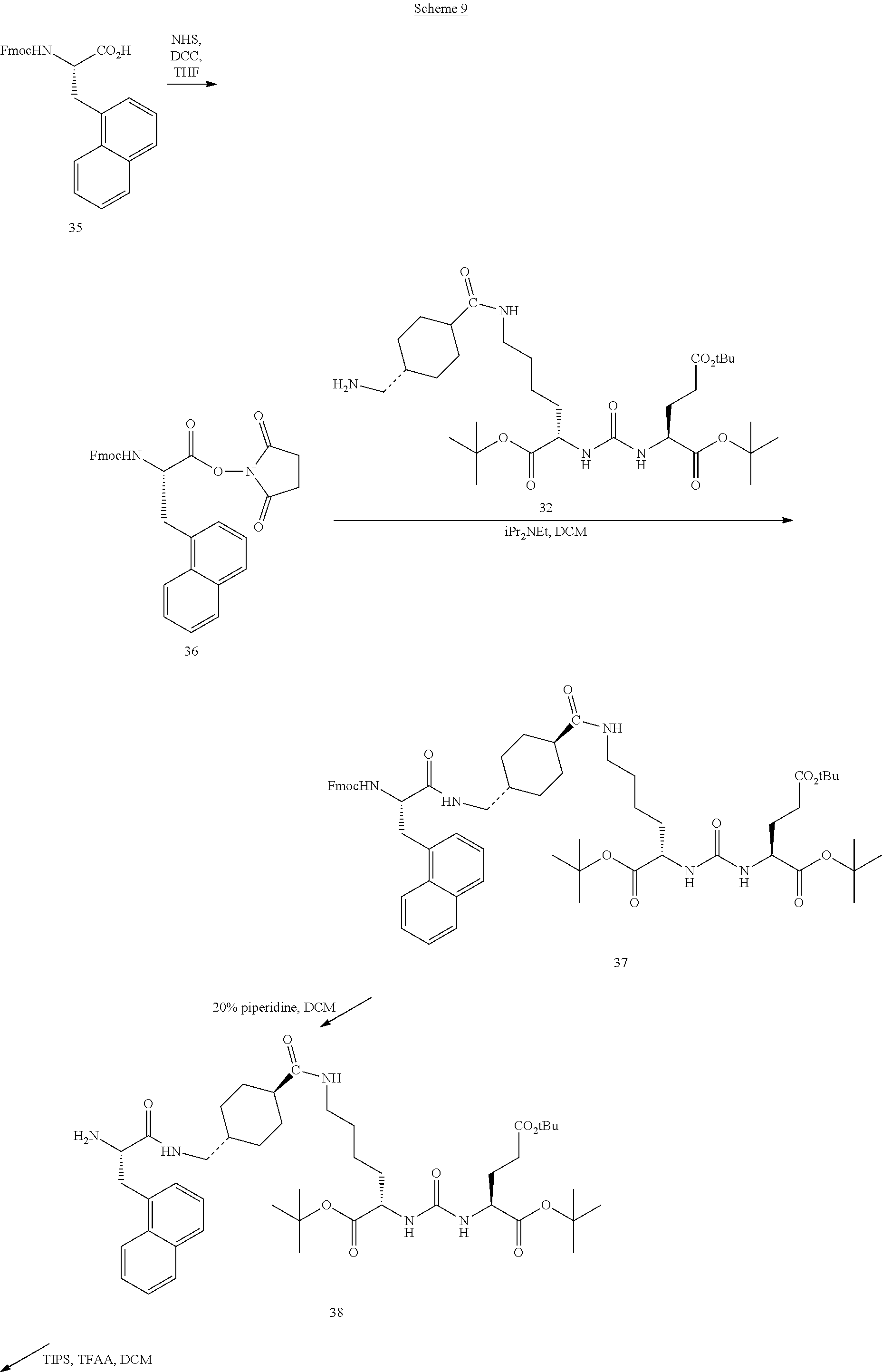

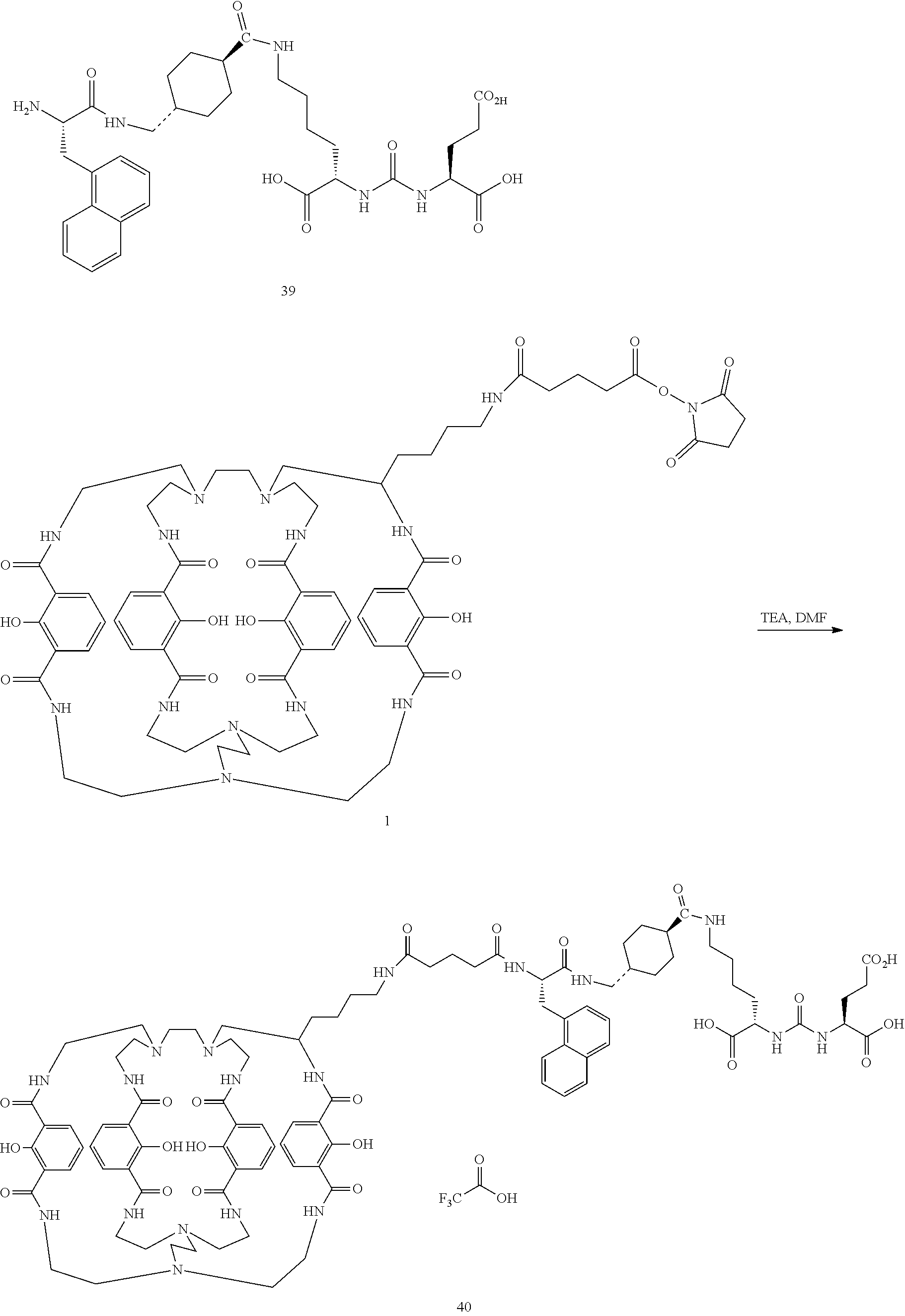

### Example 10: Synthesis of Additional Macrocyclic Metal Chelator Conjugates (Scheme 10)

In view of the above examples, one skilled in the art should be able to prepare conjugates comprised of 1-hydroxypyridine-2-oxide (1,2-HOPO) based metal chelator and site directing ligands by admixture of isothiocyanate derivative 21 and amines such as 4, 9, 24, 33, or 39. The resulting thiourea linked conjugates 41a-e are purified by HPLC.

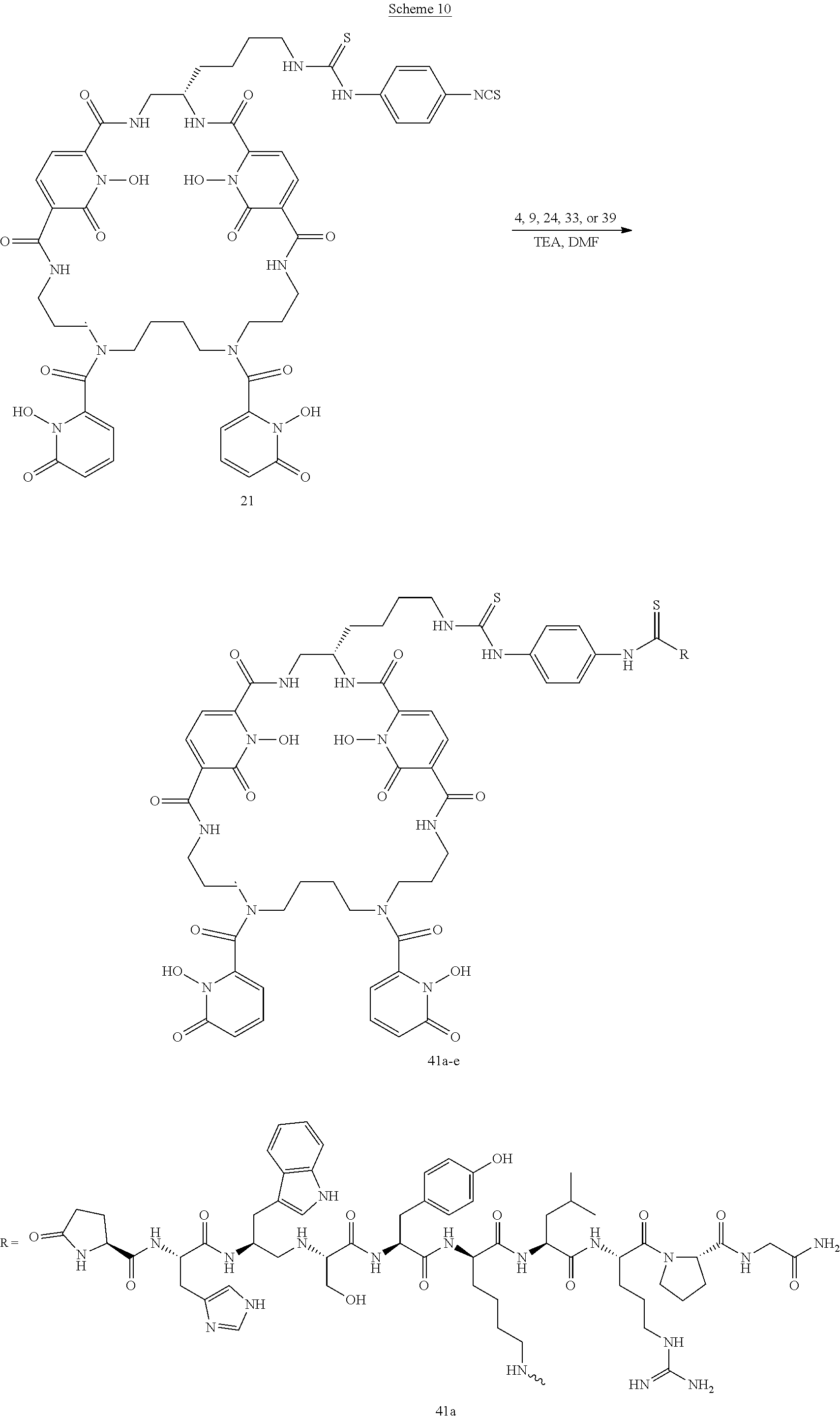

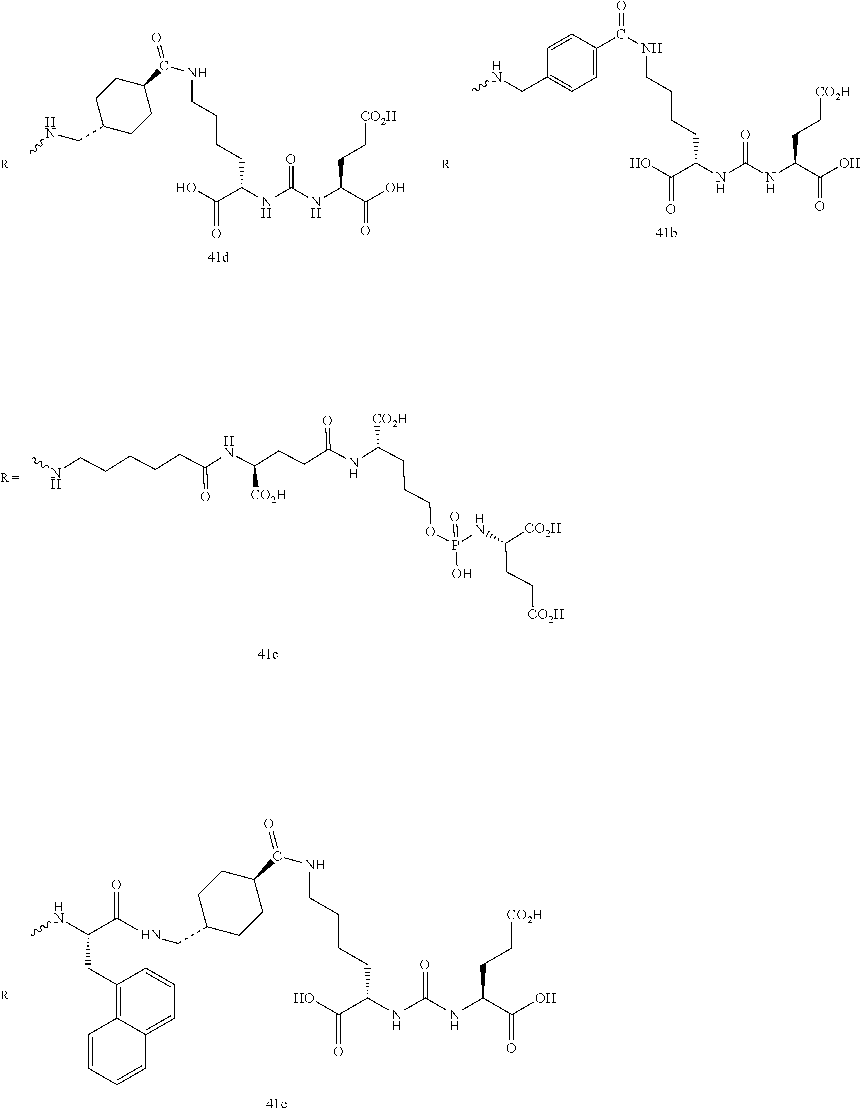

### Example 11: Synthesis of Additional Macrocyclic Metal Chelator Conjugates (Schemes 11 and 12)

An additional conjugate is prepared by reaction of 21 with amine 18, followed by deprotection using sodium hydroxide and trifluoroacetic acid to provide compound 42 (Scheme 11). An additional conjugate is prepared by reaction of 21 with 3-azido-1-propanamine to form the corresponding azide derivative 43, which is then reacted with compound 27 to form conjugate 44 (Scheme 12).

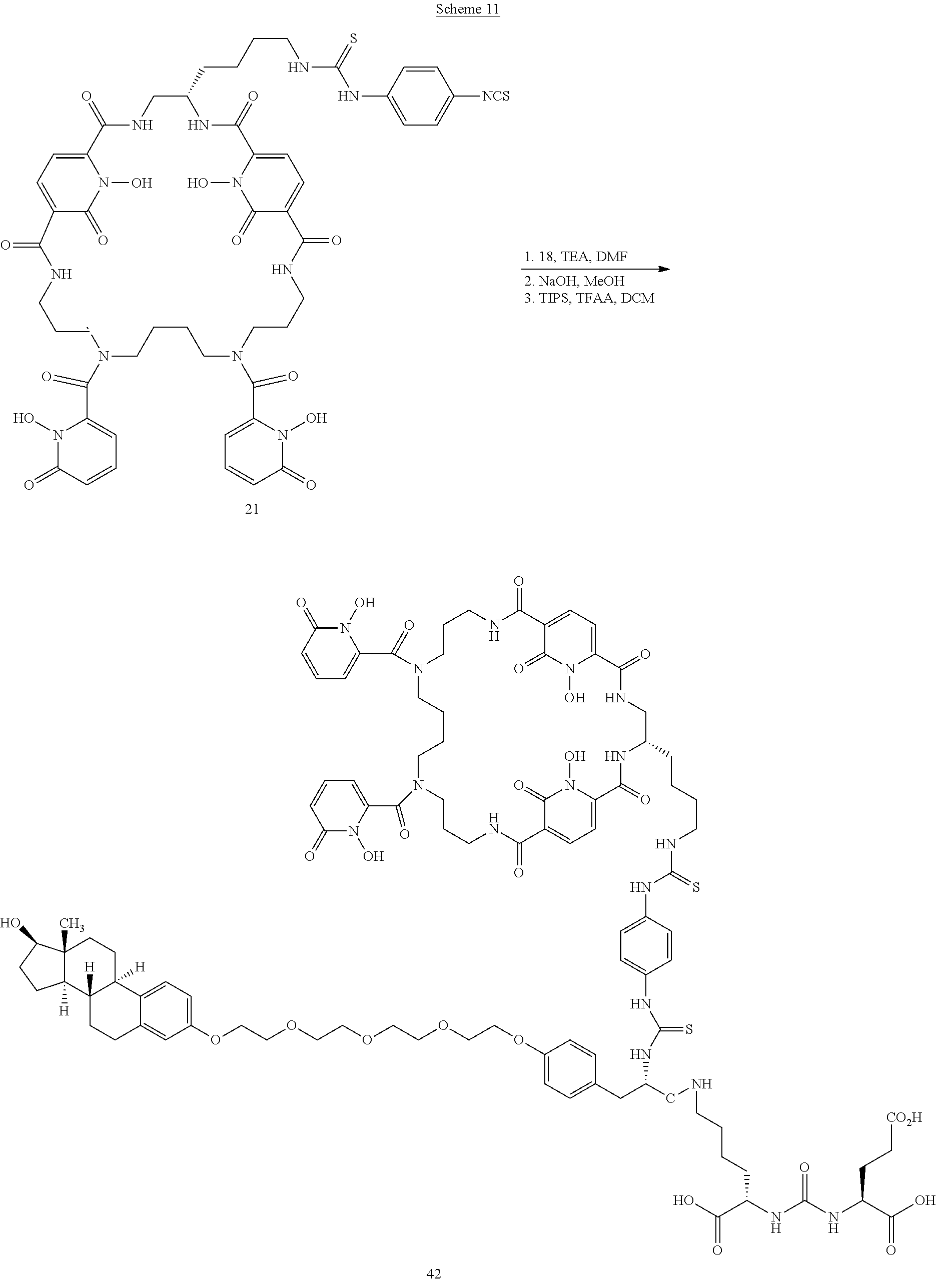

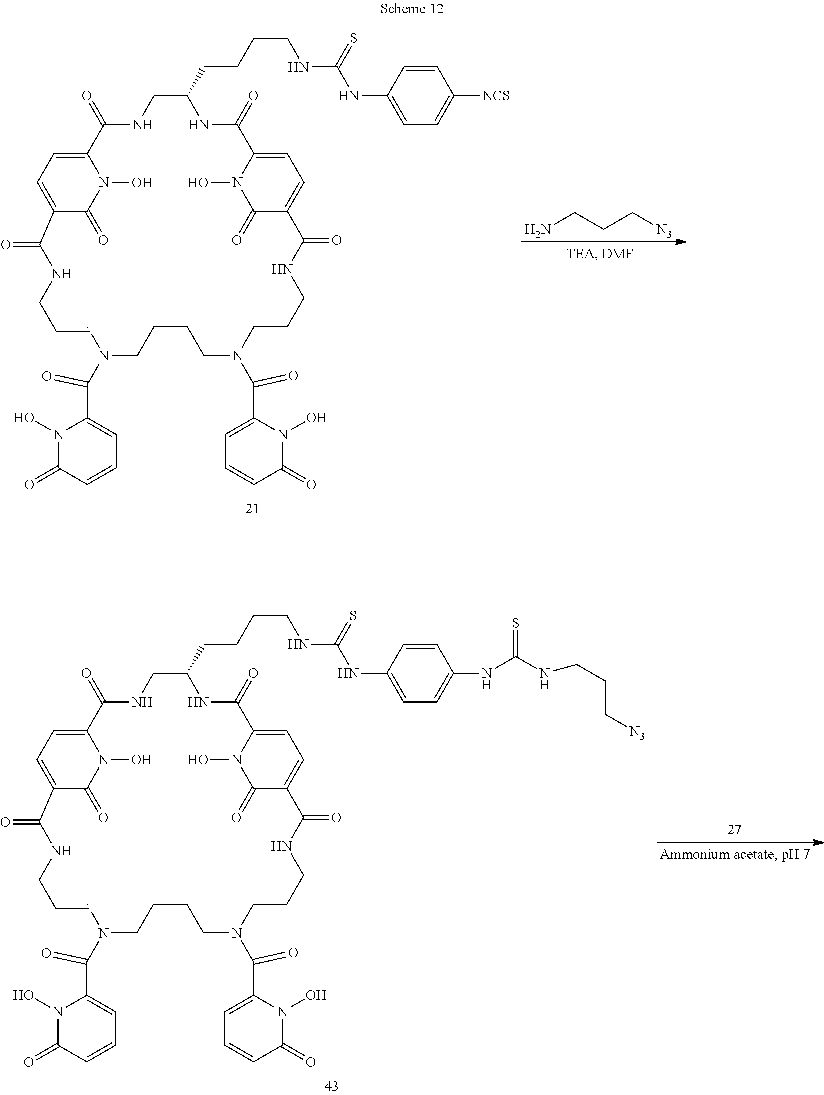

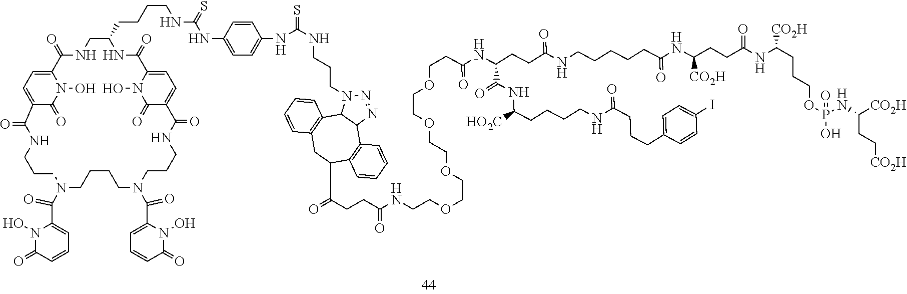 [SMILES: CCCNC(=O)c1ccc(C(=O)NC[C@H](CCCCNC(=S)Nc2ccc(NC(=S)NCCCN3N=NC4c5ccccc5C(C(=O)CCC(=O)NCCOCCOCCOCCOCCC(=O)N[C@H](CCC(=O)NCCCCCC(=O)N[C@@H](CCC(=O)N[C@@H](CCCOP(=O)(O)N[C@@H](CCC(=O)O)C(=O)O)C(=O)O)C(=O)O)C(=O)N[C@@H](CCCCNC(=O)CCCc5ccc(I)cc5)C(=O)O)Cc5ccccc5C43)cc2)NC(=O)c2ccc(C(=O)NCCCN(CCCCN(C)C(=O)c3cccc(=O)n3O)C(=O)c3cccc(=O)n3O)c(=O)n2O)n(O)c1=O]

### Example 12: Synthesis of Additional Macrocyclic Metal Chelator Conjugates (Schemes 13 and 14)

Conjugates are prepared by reaction of 21 with appropriate peptide precursors to provide compound 45a-45c (Scheme 13). Conjugates are prepared by reaction of 1 with appropriate peptide precursors to provide compound 46a-46c (Scheme 14).

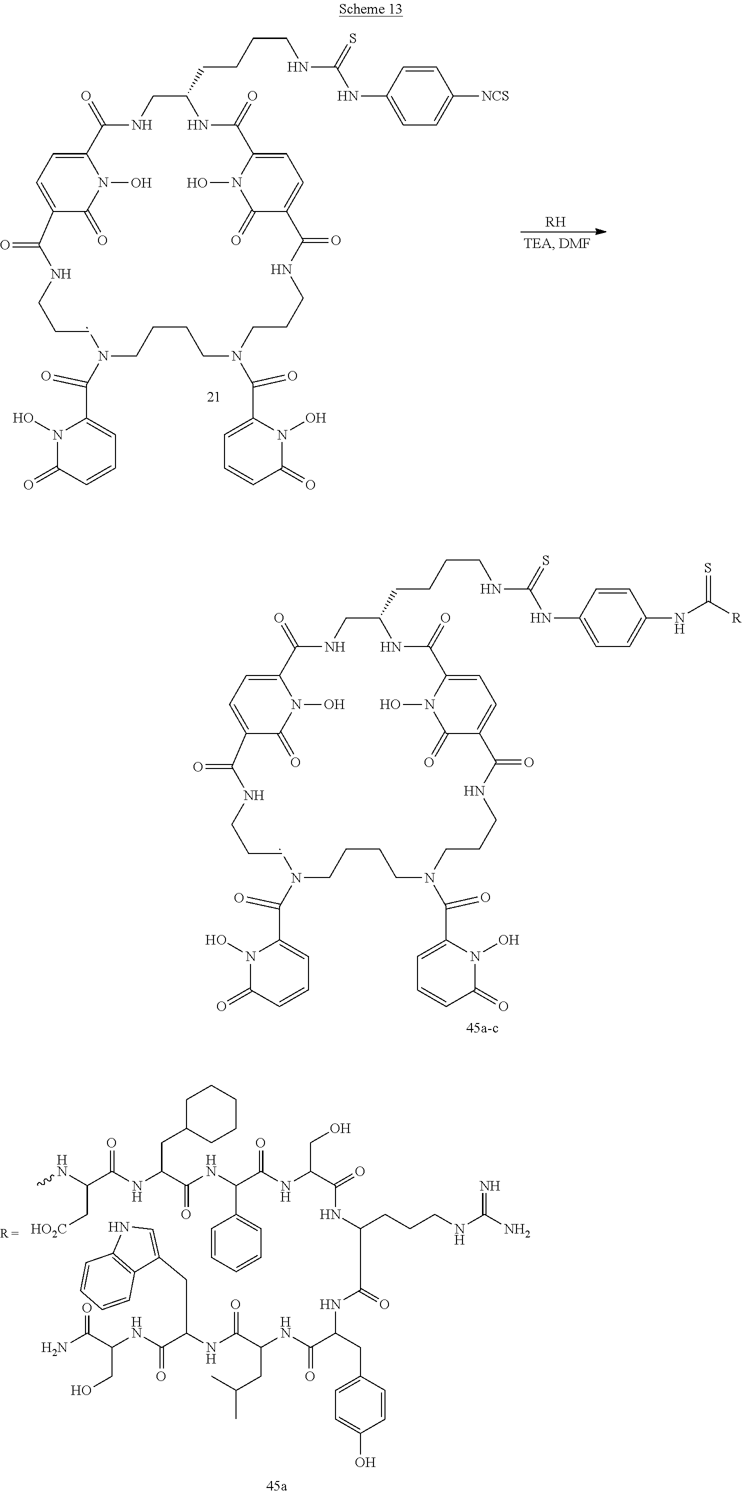

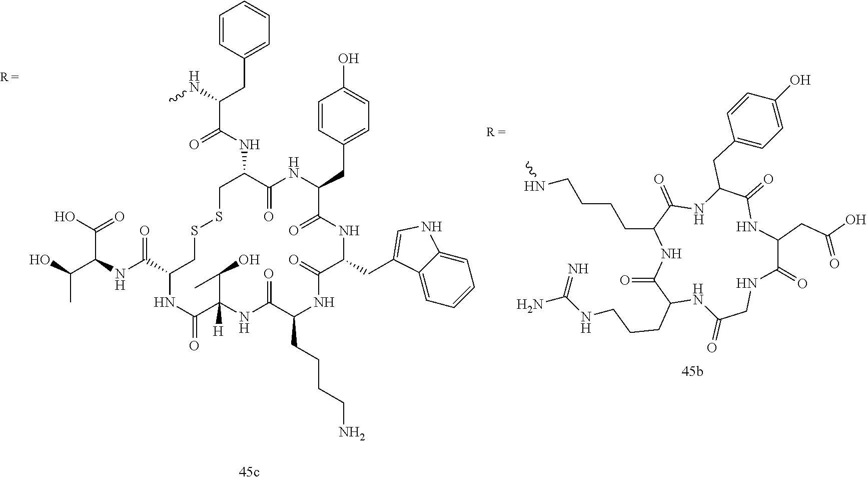

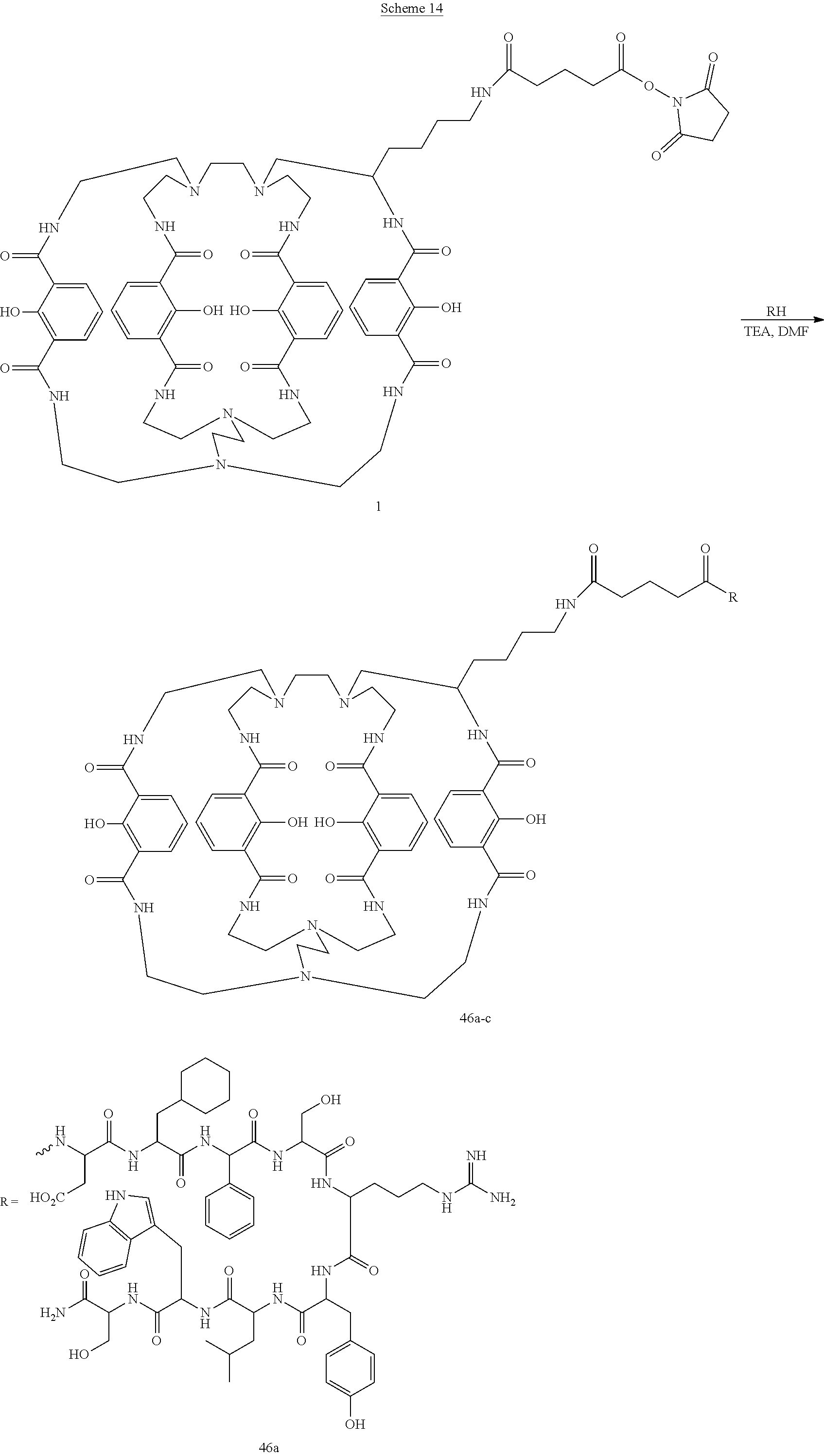

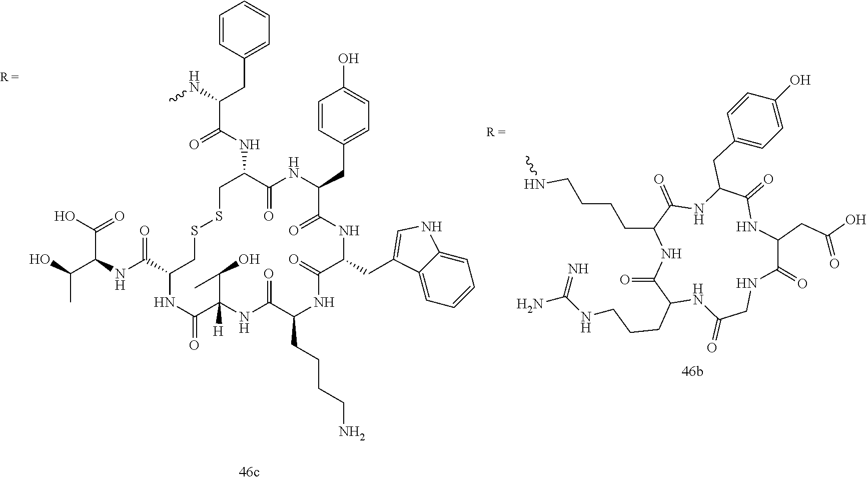

### Example 13

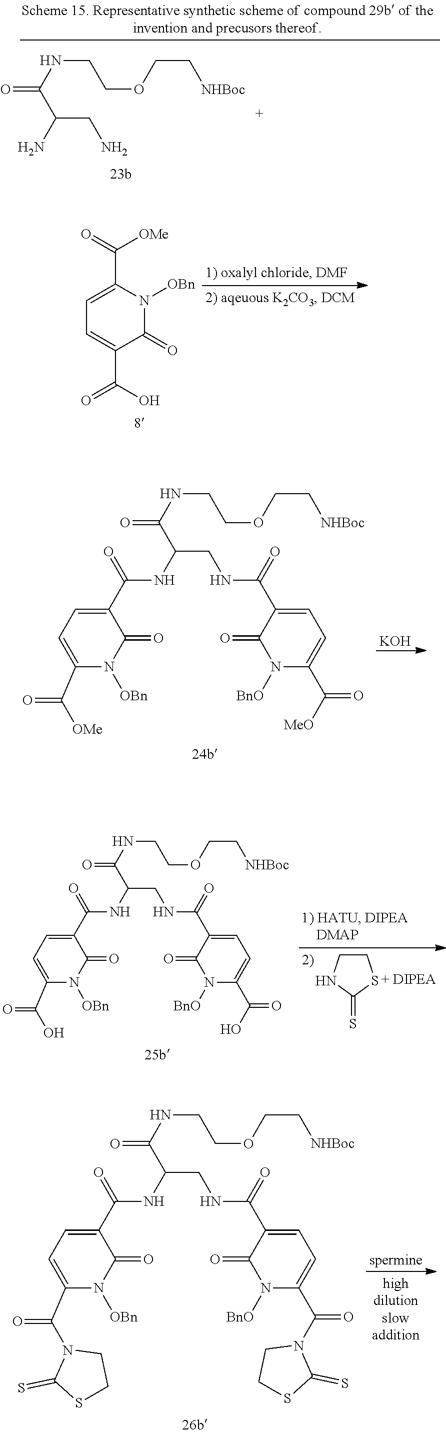

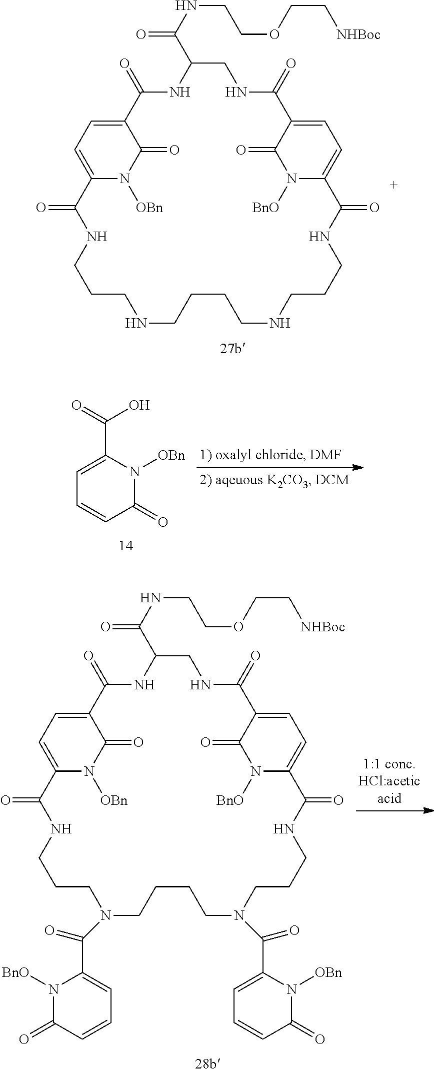 [SMILES: CC(C)(C)OC(=O)NCCOCCNC(=O)C1CNC(=O)c2ccc(n(OCc3ccccc3)c2=O)C(=O)NCCCNCCCCNCCCNC(=O)c2ccc(c(=O)n2OCc2ccccc2)C(=O)N1 ; O=C(O)c1cccc(=O)n1OCc1ccccc1 ; CC(C)(C)OC(=O)NCCOCCNC(=O)C1CNC(=O)c2ccc(n(OCc3ccccc3)c2=O)C(=O)NCCCN(C(=O)c2cccc(=O)n2OCc2ccccc2)CCCCN(C(=O)c2cccc(=O)n2OCc2ccccc2)CCCNC(=O)c2ccc(c(=O)n2OCc2ccccc2)C(=O)N1]

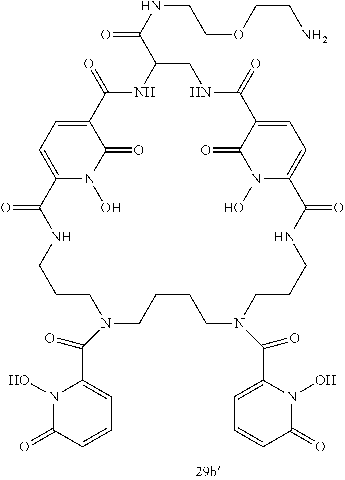 [SMILES: NCCOCCNC(=O)C1CNC(=O)c2ccc(n(O)c2=O)C(=O)NCCCN(C(=O)c2cccc(=O)n2O)CCCCN(C(=O)c2cccc(=O)n2O)CCCNC(=O)c2ccc(c(=O)n2O)C(=O)N1]

### Example 14

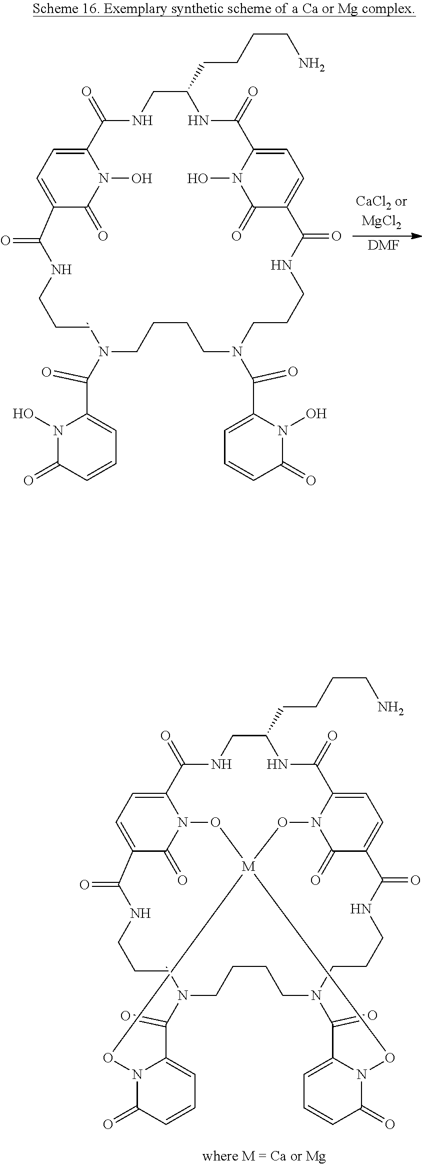

In one example, a Ca (or Mg) complex can be prepared by first mixing the chelating agent with 1.2 molar equivalents of CaCl₂) (or MgCl₂) in DMF, followed by removing the solvent under vacuum.

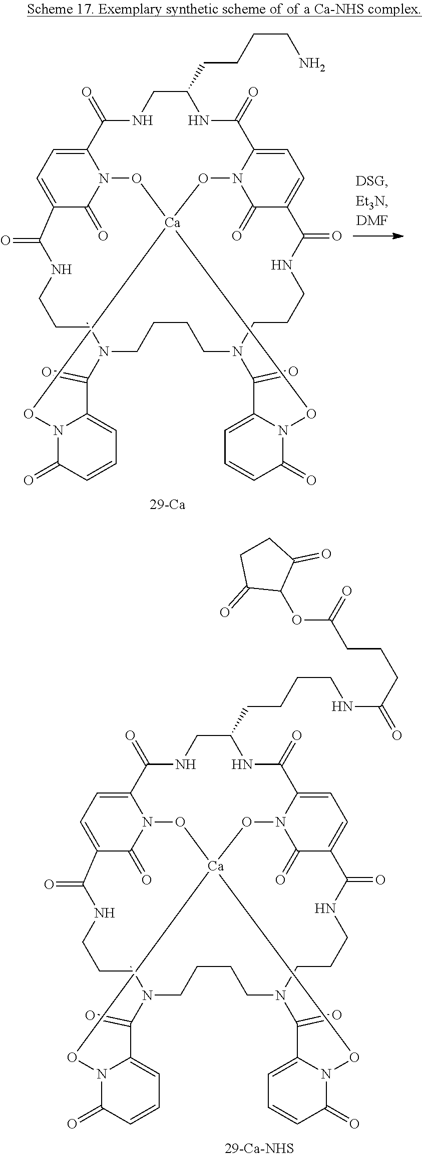

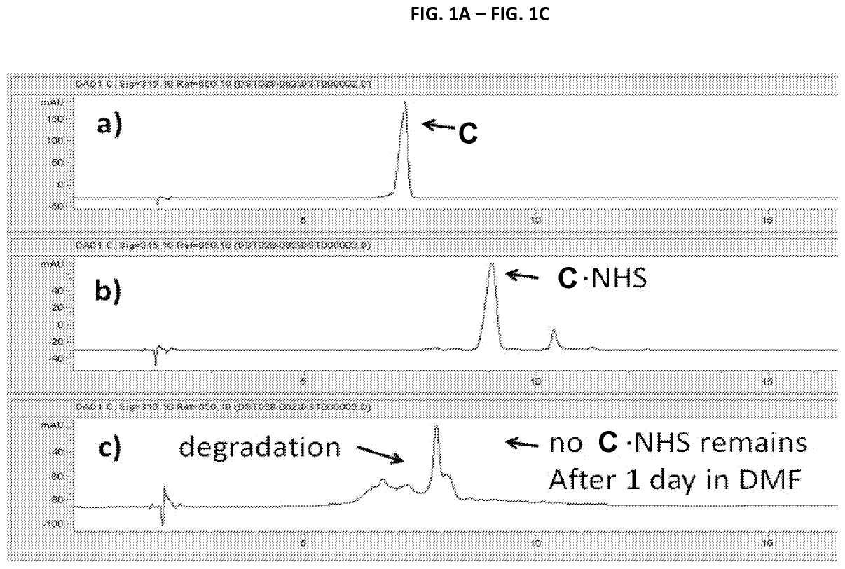

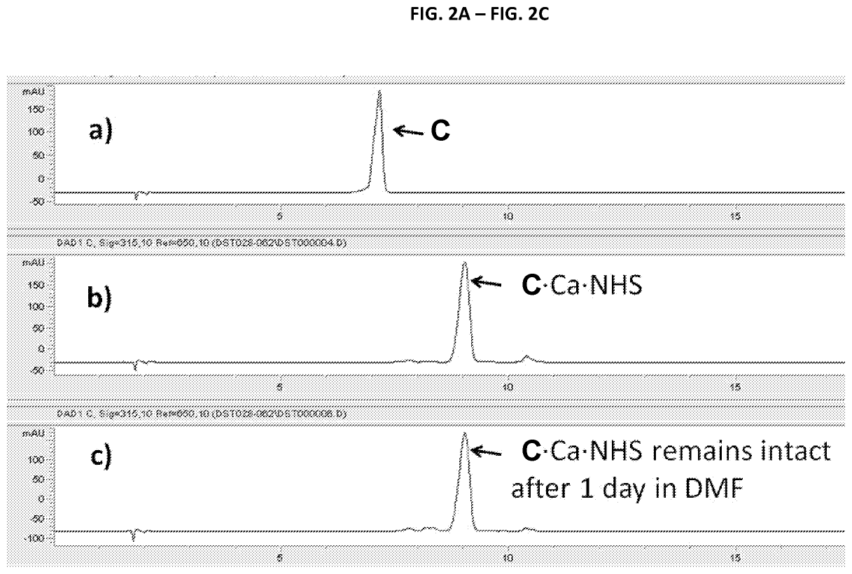

The reactive NHS esters of complexes representative Ca (or Mg) complexes can be generated by treatment of the appropriate Ca(II) (or mg(II)) complex with excess (10 eq.) di(N-succinimidyl) glutarate (DSG) in DMF and triethylamine. The reactive NHS esters are useful for attaching the metal complexes to lysine residues on proteins, or to amines of other targeting groups of interest. For example, a Ca complex can be prepared by first mixing the chelating agent with 1.2 molar equivalents of CaCl₂) in DMF, followed by removing the solvent under vacuum. The Ca complex prepared in this way can then be reacted with excess DSG in DM. Forming the Ca(II) (or Mg(II)) complex before reaction with DSG protects the chelate from reacting nonproductively with the NHS functional group. It should also be noted that Mg(II) can be used successfully for the same purpose. Both Ca(II) and Mg(II) can be readily displaced by higher valent (oxidation state III or IV) metal ions that bind more tightly than Ca(II) or Mg(II), such as lanthanides (including Eu(III) and Lu(III)), actinides (including Th(IV)), and transition metals (including Zr(IV)). The Ca(II) or Mg(II) complexes are therefore very useful for affording a conveniently reactive bifunctional chelator that can be labeled with radioisotopes of interest.
In order to demonstrate the usefulness of first forming the Ca(II) or Mg(II) complex toward preventing the degradation of the NHS analogue, we first show the results of forming an NHS product without added Ca or Mg and the resulting stability in DMF solution in the following figure. Note that all of the following HPLC chromatograms in this example are collected after adding 10 molar equivalents of EuCl₃ 5 min prior to injection for convenience of analysis.
It is clear from the HPLC data shown in FIG. 1 that, while it is possible to form and isolate NHS in the absence of Ca(II) or Mg(II), the resulting NHS product is remarkably unstable. There is no measureable amount of the desired NHS product remaining in solution once the NHS product has been dissolved in DMF and incubated at RT for one day. For comparison, the exact same experiment was performed using a Ca complex of the same chelating agent as shown in FIG. 2.
It is clear from the HPLC data that the forming a Ca complex a chelating agent yields a Ca.NHS reaction product with enhanced stability toward degradation in solution relative to the NHS reaction product. In the following figure we show the HPLC data for both the Ca.NHS and Mg.NHS reaction products after incubation in DMF at RT for 1 day, which demonstrates that Mg can be used for the same protective purpose as Ca.

### REFERENCES

Xu, J., et al., Octadentate Cages of Tb(III) 2-Hydroxyisophthalamides: A New Standard for Luminescent Lanthanide Labels. J. Am. Chem. Soc., 2011, 133, 19900-19910.
Bouvet, V., et al., Automated synthesis of [18F]DCFPyL via direct radiofluorination and validation in preclinical prostate cancer models. EJNMMI Res., 2016, 6, 40.
USPTO provisional application Ser. No. 15/914,945 (line 00228).
Ganguly, T., et al., A high-affinity [18F]-labeled phosphoramidate peptidomimetic PSMA-targeted inhibitor for PET imaging of prostate cancer. Nucl. Med. Biol., 2015, 42(10), 780-787.
Berkman, C., et al., WO2018031809.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A macrocycle having:
the structure:
CH-L⁰-TA
where CH is a chelating agent comprising one or more chelating moieties capable of binding and complexing a metal ion. An exemplary chelating agent has a formula selected from: 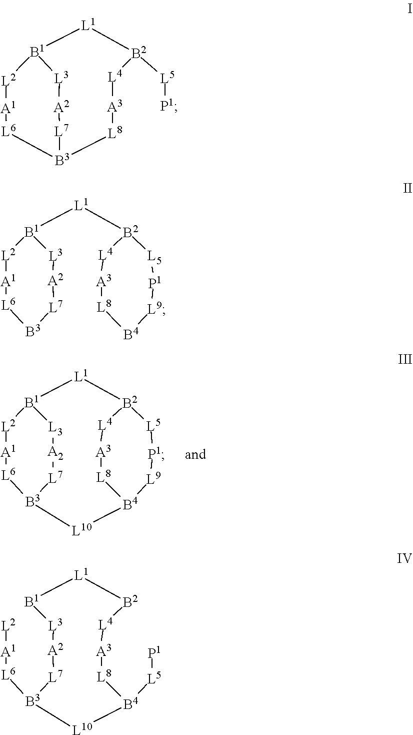
wherein
B¹, B², and B³ are independently selected from N and C;
L¹, L², L³, L⁴, L⁵, L⁶, L⁷, and L⁸ are independently selected from substituted or unsubstituted alkyl, and substituted or unsubstituted heteroalkyl;
A¹, A², and A³ are members independently selected from: 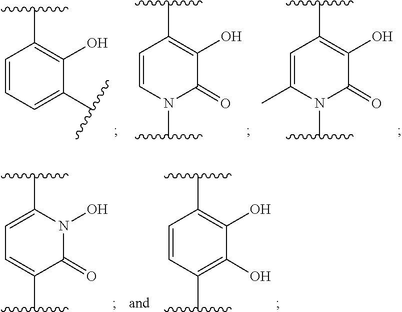
and
P¹ is a member selected from: 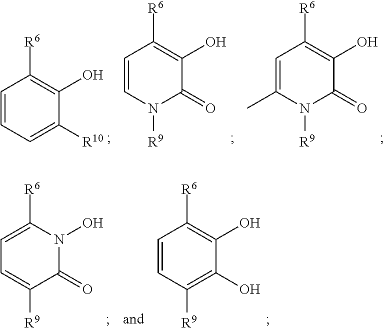
wherein R⁶, R⁹, and R¹⁰ are as defined herein, with the proviso that R⁶, R⁹, or R¹⁰ is a bond to L⁵;
L^{o} is a straight-chain or branch-chain linker joining the chelating agent (CH) to the targeting agent (TA), which is a member selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted heteroalkyl, substituted or unsubstituted heterocycloalkyl, substituted or unsubstituted aryl, substituted or unsubstituted arylalkyl, substituted or unsubstituted biaryl, substituted or unsubstituted heteroaryl, and a substituted or unsubstituted polycyclic ring system; and
TA is a targeting moiety selected from a compound binding to PSMA, LHRH, a steroid hormone, e.g., an estrogen derivative, e.g., estradiol, a somatostatin receptor binding agent and a combination thereof.

2. The macrocycle according to claim 1, wherein said macrocycle is covalently modified with at least one linker covalently attached to TA.

3. The macrocycle according to claim 2, wherein one of L¹, L², L³, L⁴, L⁵, L⁶, L⁷, L⁸, and A^{p1} is substituted with a linker covalently attached to TA.

4. The macrocycle according to claim 2, wherein said linker is attached to a targeting moiety selected from a PSMA ligand, a LHRH ligand, a somatostatin receptor binding agent or a combination thereof.

5. The macrocycle according to claim 1, wherein each of A¹, A², A³ and P¹ is independently selected from:

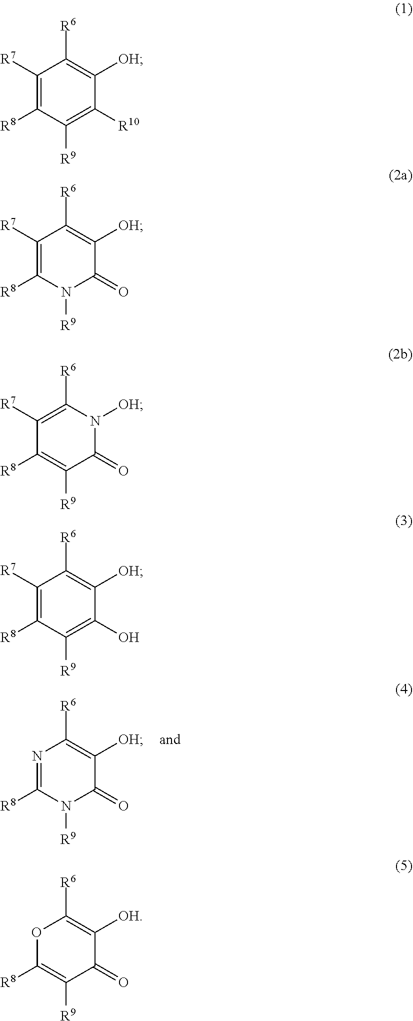

6. The macrocycle according to claim 1, wherein said macrocycle comprises one or more modifying moieties.

7. The macrocycle according to claim 6, wherein one or more of L¹, L², L³, L⁴, L⁵, L⁶, L⁷, L⁸, F¹, and P¹ comprises a modifying moiety.

8. The macrocycle according to claim 6, wherein said modifying moiety is a substituted or unsubstituted polyether.

9. The macrocycle according to claim 1, having the formula: 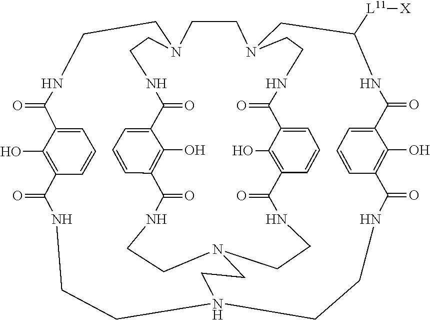
wherein L¹¹ is a linker; and
X¹ is said targeting moiety.

10. The macrocycle according to claim 1, having the formula: 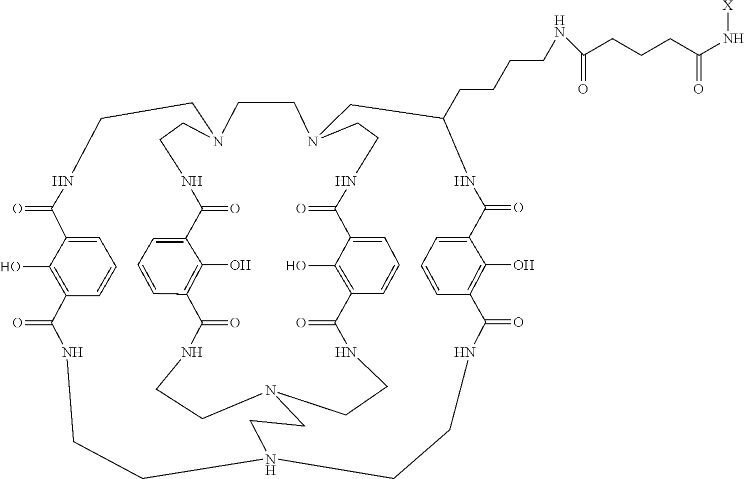
wherein X¹ is said targeting moiety.

11. A complex comprising a macrocycle according to claim 1 and a metal ion.

12. The complex according to claim 11, wherein the metal is selected from a lanthanide, an actinide, yttrium (Y), and zirconium (Zr).

13. The complex according to claim 12, wherein said lanthanide is selected from terbium (Tb), europium (Eu), dysprosium (Dy), and lutetium (Lu).

14. The complex according to claim 12, wherein said actinide is thorium (Th).

15. The complex according to claim 11, wherein said metal is a radionuclide.

16. The complex according to claim 15, wherein said metal ion is ²²⁷Th(IV) or ⁸⁹Zr(IV).
